(19) United States
(12) Patent Application Publication
(10) Pub. No.: US 2024/0085429 A1
(43) Pub. Date: Mar. 14, 2024

(54) ALTERING PROTEIN FUNCTION BY PHARMACOLOGICAL TARGETING OF MEMBRANE DOMAINS

(71) Applicants: University of Virginia Patent Foundation, Charlottesville, VA (US); Vanderbilt University, Nashville, TN (US)

(72) Inventors: Anne Kathleen Kenworthy, Charlottesville, VA (US); Nico Fricke, Charlottesville, VA (US); Ajit Tiwari, Nashville, TN (US); Charles R. Sanders, Nashville, TN (US); Krishnan Raghunathan, Nashville, TN (US); Hui Huang, Nashville, TN (US); Ricardo F. Capone, Nashville, TN (US); Joshua A. Bauer, Nashville, TN (US); Katherine M. Stefanski, Nashville, TN (US)

(73) Assignees: University of Virginia Patent Foundation, Charlottesville, VA (US); Vanderbilt University, Nashville, TN (US)

(21) Appl. No.: 18/273,728

(22) PCT Filed: Jan. 24, 2022
(86) PCT No.: PCT/US2022/013573

§ 371 Date: Jul. 21, 2023

Related U.S. Application Data
(60) Provisional application No. 63/140,603, filed on Jan. 22, 2021.

(51) Int. Cl. G01N 33/58; G01N 21/64; G01N 33/92

(52) U.S. Cl. CPC G01N 33/582; G01N 21/6428; G01N 33/92; G01N 2021/6441; G01N 2500/20

(57) ABSTRACT

Disclosed are methods, including automated methods, for identifying a compound that impacts a characteristic of a lipid raft phase domain, a characteristic of a non-raft phase domain, and/or a characteristic of one or more membrane proteins. In some embodiments, the method comprises contacting a population of vesicles with a candidate compound, wherein a population of vesicles, wherein one or more vesicles in the population of optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are phase separated; detecting a signal from the population of vesicles; and identifying the candidate compound as having an impact on a characteristic of a lipid raft phase domain, a characteristic of a non-raft phase domain, and/or a characteristic of one or more membrane proteins based on the signal. Systems and non-transitory computer readable medium comprising computer executable instructions embodied in a computer readable medium that when executed by a processor of a computer control the computer to perform steps of the method are also disclosed.

### CROSS REFERENCE TO RELATED APPLICATION

The presently disclosed subject matter claims the benefit of U.S. Provisional Patent Application Ser. No. 63/140,603, filed Jan. 22, 2021, the disclosure of which incorporated herein by reference in its entirety.

### GOVERNMENT INTEREST

This invention was made with government support under Grant Nos. GM138493, AG056147, NS095989, and GM106720 awarded by The National Institutes of Health. The government has certain rights in the invention.

### BACKGROUND

Membrane rafts, also referred to as lipid rafts, represent an extensively studied yet persistently enigmatic example of how membrane organization can modulate cellular function (Sezgin et al., 2017). Typically defined as nanoscopic cholesterol-enriched domains that share properties with liquid ordered domains in vitro, rafts co-exist with disordered domains in cell membranes and regulate numerous cellular functions by controlling the interaction partners and proximal membrane environment of associated proteins (Sezgin et al., 2017). Consistent with their extensive biological roles, rafts have been implicated in a variety of normal physiological processes as well as pathological conditions (Verma, 2009; Hryniewicz-Jankowska et al., 2014; Sezgin et al., 2017; Bernardes & Fialho, 2018; Bukrinsky et al., 2020; Sviridov et al., 2020). Raft-dependent processes are typically identified and manipulated by altering membrane lipid composition, most commonly via cholesterol depletion (Sezgin et al., 2017). However, such approaches can have significant pleiotropic effects and non-specifically affect multiple lipid-dependent pathways (Zidovetzki & Levitan, 2007; Levental & Levental, 2015).

Another possible, though not yet widely explored approach to modulate rafts in biological membranes is through the use of small molecules (Verma, 2009; Hryniewicz-Jankowska et al., 2014; Bernardes & Fialho, 2018; Sviridov et al., 2020; Tsuchiya & Mizogami, 2020). For example, specific therapeutics that can modulate microdomains are being actively considered for development (Sviridov et al., 2020). Besides targeting specific lipids that alter receptor signaling, certain drugs can also modulate general properties of membrane rafts (Zhou et al., 2012; Gray et al., 2013). Furthermore, in cancer cell lines, changes in membrane heterogeneity is correlated with resistance to chemotherapeutics, and altering membrane heterogeneity can modulate cellular responses to chemotherapeutic drugs (Raghunathan et al., 2015). Thus, chemically-based strategies could potentially provide new experimental tools to manipulate rafts in vitro and even eventually establish new therapeutics for human diseases linked to raft biology. However, systematic screens to identify small molecules that target the formation and properties of rafts have not been described.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to a method for identifying a compound that impacts a characteristic of a lipid raft phase domain, a characteristic of a non-raft phase domain, and/or a characteristic of one or more membrane proteins. In some embodiments, the method comprises contacting a population of vesicles, wherein one or more vesicles in the population of vesicles optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are phase separated; detecting a signal from the population of vesicles; and identifying the candidate compound as having an impact on a characteristic of a lipid raft phase domain, a characteristic of a non-raft phase domain, and/or a characteristic of one or more membrane proteins. In some embodiments, the method is an automated method.

In some embodiments, the signal is used to assess whether the candidate compound induces changes in the percent of vesicles containing the single visible phase within the population of vesicles; changes in the relative sizes and/or characteristics of the lipid raft phase domain versus the non-raft phase domain; and/or changes in the distribution of one or more membrane proteins, such as between the lipid raft phase domain and the non-raft phase domain. In some embodiments, the characteristic of a lipid raft phase domain, a characteristic of a non-raft phase domain, and/or a characteristic of one or more membrane proteins comprises membrane phase behavior, partitioning of proteins between the lipid raft phase domain and the non-raft phase domain, or both.

In some embodiments, the vesicle comprises a Giant Plasma-Membrane Derived Vesicle (GPMV). In some embodiments, the lipid raft phase domain, the non-raft phase domain, and/or the one or more membrane proteins are distinguishably labeled. In some embodiments, (a) the lipid raft phase domain is labeled with a first fluorescent label and the non-raft phase domain is labeled with a second fluorescent label, wherein the first fluorescent label and the second fluorescent label are distinguishable from each other; (b) the lipid raft phase domain is labeled with a first fluorescent label and the one or more membrane proteins is labeled with one or more additional labels, wherein the first fluorescent label and the one or more additional fluorescent labels are all distinguishable from each other; (c) the non-raft phase domain is labeled with a first fluorescent label and the one or more membrane proteins is labeled with one or more additional fluorescent labels, wherein the first fluorescent label and the additional fluorescent labels are all distinguishable from each other; and (d) the lipid raft phase domain is labeled with a first fluorescent label, the non-raft phase domain is labeled with a second fluorescent label, and the one or more membrane proteins is labeled with a third or more fluorescent label, wherein the first fluorescent label, the second fluorescent label, and the third or more fluorescent labels are all distinguishable from each other.

In some embodiments, detecting a signal in the population of vesicles further comprises detecting a detectable label in the population of vesicles, taking one or more images of one or more vesicles in the population of vesicles, or a combination thereof. In some embodiments, detecting a signal from the population of vesicles comprises identifying one or more vesicles, excluding one or more vesicles that do not meet one or more selection criteria, mapping a position of membrane contour for each of the one or more vesicles, generating a plot of fluorescence intensity along the membrane of the vesicle, calculating a partition coefficient of a vesicle, scoring a vesicle as phase separated or not, or any combination of any of the foregoing.

In some embodiments, calculating a partition coefficient of a vesicle comprises calculating the partition coefficients of the fluorescent labels and/or fluorescently labeled membrane proteins as a function of angle around the entire vesicle. In some embodiments, scoring a vesicle as phase separated or not comprises determining whether a histogram of the angular partition coefficient of a vesicle comprises a single peak centered around 0.5 or instead contains multiple peaks reflecting a presence of fluorophore-rich and fluorophore-poor domains. In some embodiments, scoring a vesicle as phase separated or not comprises separating data points into two separate quadrants for vesicles having phase separated membranes and colocalizing datapoints on a diagonal for membranes containing a single uniform phase.

In some embodiments, the method further comprises correcting the imaging for a position effect.

In some embodiments, identifying the candidate compound as having an impact on a characteristic of a lipid raft based on the signal comprises detecting a change in phase separation in one or more vesicles in the population of vesicles, such as a change from a single detectable phase to phase separated between the lipid raft phase domain and the non-raft phase domain, optionally wherein the change is detected by comparing the signal with a signal from the population of vesicles detected before the contacting with the candidate compound; and/or detecting a change in at least one of the one or more membrane proteins from the lipid raft phase domain or the non-raft phase domain to the other phase domain, optionally wherein the change is detected by comparing the signal with a signal from the population of vesicles detected before the contacting with the candidate compound; and/or calculating Z-scores and ranking hits. In some embodiments, detecting a change in phase separation comprises detecting a change in partition coefficients and/or a change in percent of phase separated vesicles in the population of vesicles. In some embodiments, the measured partition coefficient is for the distribution of a fluorescently-labeled protein between lipid raft and non-raft phase domains.

In some embodiments, the method comprises analyzing multiple vesicles in the population of vesicles across multiple reaction wells in an automated fashion.

In some embodiments, a system for identifying the candidate compound as having an impact on a characteristic of a lipid raft phase domain, a characteristic of a non-raft phase domain, and/or a characteristic of one or more membrane proteins is provided. In some embodiments, the system comprises a plate comprising a plurality of reaction wells for contacting a population of vesicles, wherein one or more vesicles in the population of vesicles optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are phase separated; and a computing platform including a processor and memory, the computing platform including a module configured to detect a signal from the population of vesicles using a microscope, camera or a sensor communicatively coupled to the computing platform; and to identify the candidate compound as having an impact on a characteristic of a lipid raft phase domain, a characteristic of a non-raft phase domain, and/or a characteristic of one or more membrane proteins based on the signal. In some embodiments, the system is configured to function automatedly.

In some embodiments, the signal is used to assess whether the candidate compound induces changes in the percent of vesicles containing the single visible phase within the population of vesicles; changes in the relative sizes and/or characteristics of the lipid raft phase domain versus the non-raft phase domain; and/or changes in the distribution of one or more membrane proteins between the lipid raft phase domain and the non-raft phase domain. In some embodiments, the characteristic of a lipid raft phase domain, a characteristic of a non-raft phase domain, and/or a characteristic of one or more membrane proteins comprises membrane phase behavior, partitioning of proteins between the lipid raft phase domain and the non-raft phase domain, or both. In some embodiments, the vesicle comprises a Giant Plasma-Membrane Derived Vesicle (GPMV).

In some embodiments, the lipid raft phase domain, the non-raft phase domain, and/or the one or more membrane proteins are distinguishably labeled. In some embodiments, (a) the lipid raft phase domain is labeled with a first fluorescent label and the non-raft phase domain is labeled with a second fluorescent label, wherein the first fluorescent label and the second fluorescent label are distinguishable from each other; (b) the lipid raft phase domain is labeled with a first fluorescent label and the one or more membrane proteins is labeled with one or more additional fluorescent labels, wherein the first fluorescent label and the one or more additional fluorescent labels are all distinguishable from each other; (c) the non-raft phase domain is labeled with a first fluorescent label and the one or more membrane proteins is labeled with one or more additional fluorescent labels, wherein the first fluorescent label and the one or more additional fluorescent labels are all distinguishable from each other; and (d) the lipid raft phase domain is labeled with a first fluorescent label, the non-raft phase domain is labeled with a second fluorescent label, and the one or more membrane proteins is labeled with a third or more fluorescent label, wherein the first fluorescent label, the second fluorescent label, and the third or more fluorescent labels are all distinguishable from each other.

In some embodiments, detecting a signal in the population of vesicles further comprises detecting a detectable label in the population of vesicles, taking one or more images of one or more vesicles in the population of vesicles, or a combination thereof. In some embodiments, detecting a signal from the population of vesicles comprises identifying one or more vesicles, excluding one or more vesicles that do not meet one or more selection criteria, mapping a position of membrane contour for each of the one or more vesicles, generating a plot of fluorescence intensity along the membrane of the vesicle, calculating a partition coefficient of a vesicle, scoring a vesicle as phase separated or not, or any combination of any of the foregoing.

In some embodiments, calculating a partition coefficient of a vesicle comprises calculating the partition coefficients of the fluorescent labels and/or fluorescently labeled membrane proteins as a function of angle around the entire vesicle. In some embodiments, scoring a vesicle as phase separated or not comprises determining whether a histogram of the angular partition coefficient of a vesicle comprises a single peak centered around 0.5 or instead contains multiple peaks reflecting a presence of fluorophore-rich and fluorophore-poor domains. In some embodiments, scoring a vesicle as phase separated or not comprises separating data points into two separate quadrants for vesicles having phase separated membranes and colocalizing datapoints on a diagonal for membranes containing a single uniform phase. In some embodiments, the computing platform is configured for correcting the imaging for a position effect.

In some embodiments, identifying the candidate compound as having an impact on a characteristic of a lipid raft based on the signal comprises detecting a change in phase separation in one or more vesicles in the population of vesicles between the lipid raft phase domain and the non-raft phase domain, optionally wherein the change is detected by comparing the signal with a signal from the population of vesicles detected before the contacting with the candidate compound; and/or detecting a change in at least one of the one or more membrane proteins from the lipid raft phase domain or the non-raft phase domain to the other phase domain, optionally wherein the change is detected by comparing the signal with a signal from the population of vesicles detected before the contacting with the candidate compound; and/or calculating Z-scores and ranking hits,

In some embodiments, detecting a change in phase separation comprising detecting a change in partition coefficients and/or a change in percent of phase separated vesicles in the population of vesicles. In some embodiments, the measured partition coefficient is for the distribution of a fluorescently-labeled protein between lipid raft and non-raft phase domains. In some embodiments, the system is configured to analyze multiple vesicles in the population of vesicles across multiple reaction wells in an automated fashion.

In some embodiments, provided is one or more non-transitory computer readable media having stored thereon executable instructions that when executed by one or more processors of one or more computers control the one or more computers to perform steps for identifying the candidate compound as having an impact on a characteristic of a lipid raft phase domain, a characteristic of a non-raft phase domain, and/or a characteristic of one or more membrane proteins. In some embodiments, the steps comprise: contacting a population of vesicles, wherein one or more vesicles in the population of vesicles optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are phase separated; detecting a signal from the population of vesicles; and identifying the candidate compound as having an impact on a characteristic of a lipid raft phase domain, a characteristic of a non-raft phase domain, and/or a characteristic of one or more membrane proteins based on the signal. In some embodiments, the steps are for automatedly identifying the candidate compound as having an impact a characteristic of a lipid raft phase domain, a characteristic of a non-raft phase domain, and/or a characteristic of one or more membrane proteins.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

Accordingly, it is an object of the presently disclosed subject matter to provide methods, systems, and computer readable media for identifying a compound that impacts a characteristic of a lipid raft phase domain, a characteristic of a non-raft phase domain, and/or a characteristic of one or more membrane proteins. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIGS. 1A to 1J. Principles underlying the high content imaging screen and data analysis pipeline. (FIG. 1A) The addition of small molecules can hypothetically enhance (far right dashed line; magenta line when shown in color), impede (far left dashed line; green line when shown in color), or have no effect on (solid black line) the ability of GPMVs to undergo phase separation. These outcomes can be distinguished by comparing the percentage of phase separated vesicles observed under control and experimental conditions at a single temperature (dotted vertical line; blue line when shown in color). (FIGS. 1B-1D) Representative high content image of HeLa cell-derived GPMVs labeled with DiD (magenta when shown in color) and NBD-DSPE (green when shown in color) shown at increasingly higher magnifications. Scale bars, 20 μm in FIG. 1B, 20 μm in FIG. 1C, and 5 μm in FIG. 1D. (FIG. 1E-1J) Example of how angular phase preference coefficients p_{ang}(ϕ) are measured as a function of angle ϕ and used to discern single phase and phase separated GPMVs. (FIG. 1E-1G) Example of a single phase GPMV, together with a plot and histograms of its corresponding angular phase preference coefficients. (FIG. 1H-FIG. 1J) Example of a phase separated GPMV, together with a plot and histograms of its angular phase preference coefficients. Scale bars in FIG. 1E and FIG. 1H, 5 μm. FIG. 1J, upper panel DiD; lower panel NBD-DSPE.

FIGS. 2A to 2F. Results and validation of HTS for compounds that impact GPMV phase behavior. (FIGS. 2A, 2B) Histograms of (FIG. 2A) percent phase separated vesicles and (FIG. 2B) P_{ordered} for NBD-DSPE across all wells for one representative screen. (FIG. 2C) Z-scores for percentage of phase separated vesicles across three independent screens performed on different days to test reproducibility. Vertical lines demarcate the position of individual plates in each screen. Each datapoint corresponds to measurements from an individual well (single compound). Compounds with Z scores of greater than 2 are shown as open squares and compounds with Z scores of less than −2 are shown as open circles (FIG. 2D) Plot comparing the average Z-scores for P_{ordered} of NBD-DSPE versus the average Z-scores for the percentage of phase separated vesicles versus for each compound across all screens. The identities of several hits are indicated on the graph. (FIG. 2E) Dose response curves for two representative hits, TLCK and C6-ceramide. Data represent the mean±SD from 2 independent experiments performed in duplicate. Insets show structures of TLCK and C6-ceramide. (FIG. 2F) Impact of C6-ceramide and TLCK on DT_{misc}. See FIG. 9 for examples of curves used to calculate ΔT_{misc}. Data represent the mean±SD from 2 independent experiments. Abbreviations in FIG. 2D are as follows: TOFA, 5-(tetradecyloxy)-2-furancarboxylic acid; 2-thio-PAF, 1-O-hexadecyl-2-deoxy-2-thio-S-acetyl-sn-glyceryl-3-phosphorylcholine; TLCK, Na-tosyl-L-lysyl chloromethyl ketone; EGCG, (−)-epigallocatecatchin gallate; N-St. taurine, N-stearoyl taurine; 2-O-methyl PAF 16, 1-O-hexadecyl-2-O-methyl-sn-glyceryl-3-phosphorylcholine.

FIGS. 3A to 3E. Workflow and system for image analysis and data processing from high throughput screen. (FIG. 3A) Representative method; (FIG. 3B) Vesicle detection and selection. (FIG. 3C) Analysis of individual vesicles. (FIG. 3D) Statistical analysis performed across populations of vesicles; (FIG. 3E) Representative system.

FIGS. 4A to 4C. Automated vesicle selection process. (FIG. 4A) Zooms of a portion of the image of GPMVs shown in FIG. 1B. (FIG. 4B) Disc-like structures identified during the first round of automated image analysis are highlighted by white circles. (FIG. 4C) GPMVs that pass all exclusion criteria and are included in the final analysis are highlighted in white circles. Note that due to the high interior fluorescence signal, GPMVs labeled with DiD often do not pass all selection criteria, especially when the membrane signal is weak and uniform. This effect leads to an undercounting of non-phase separated vesicles, and consequently an overestimation of the percentage of phase separated vesicles as reported by the DiD fluorescence signals (Table 3). Examples of double-labeled GPMVs where the GPMV is detected in the NBD-DSPE channel but not in the DiD channel are highlighted with arrows. Scale bar, 20 μm.

FIGS. 5A to 5E. Contour detection and refinement of membrane position of individual GPMVs. (FIG. 5A) A radial scan is performed for each found disc to find the maxima and estimate the membrane position for both dyes. The arrow shows an example of the line used to perform the radial scan. (FIG. 5B) A radial profile plot is used to identity the fluorescence peak corresponding to the position of the membrane in the DiD channel (circles, shown as magenta circles when in color) and NBD-DSPE channel (squares, shown as green squares when in color). (FIG. 5C) Found position of the membrane contour (white dots) and background (solid line) for the GPMV shown in FIG. 5A. (FIG. 5D) To refine the position of the membrane in each vesicle, a circle (dotted white line) is fit into the contours identified in the initial scan shown in FIG. 5C. Arrow indicates angle=0° (FIG. 5E) Intensity profiles as a function of angle along the membrane are then generated. For consistency, GPMVs are identical to the ones shown in FIGS. 1A to 1J. Scale bar, 5 μm.

FIG. 6A to 6D. The percentage of phase separated vesicles in the DiD channel is systematically overestimated in wide field images due to the under-detection of DiD-labeled vesicles in the data analysis pipeline. GPMVs derived from HeLa cells were labeled with NBD-DSPE and DiD and imaged via (FIG. 6A) widefield microscopy or (FIG. 6B) spinning disc confocal microscopy. Images are from the same plate of GPMVs. High internal fluorescence intensity of red channel seen in the wide field images (white arrows) is not present in the confocal images. Scale bar, 50 μm. (FIG. 6C) Confocal and widefield images were analyzed in VesA. VesA detects more usable vesicles in the red channel in confocal images. (FIG. 6D) VesA overestimates the percentage of phase separated vesicles in DiD-labeled widefield images. This overestimation is eliminated when confocal images are analyzed. Data are shown for 9 fields of GPMVs.

FIG. 7. TLCK and C6-ceramide alter P_{ordered} for NBD-DSPE in a dose-dependent manner. Data from the same experiments shown in FIG. 2E were plotted to show the dependence of P_{ordered} for NBD-DSPE on the concentration of TLCK (circles, blue circles when shown in color) or C6-ceramide (squares, red squares when shown in color). Data were normalized to reflect relative changes and represent the mean±SD from 2 independent experiments run in duplicate.

FIG. 8. DMSO has no observable effect on membrane phase behavior over the range of concentrations used in this study. GPMVs derived from HeLa cells were incubated with the indicated concentration of DMSO prior to imaging at RT. Plots shows the percentage of phase separated vesicles (black circles) and P_{ordered} for NBD-DSPE (squares, magenta squares when shown in color) as a function of DMSO concentration. Each datapoint represents the mean±SD. Data are from a single experiment performed in triplicate.

FIG. 9. Representative temperature scan illustrating the effect of TLCK on T_{misc}. HeLa cell-derived GPMVs were incubated with 10 μM TLCK (squares, magenta squares when shown in color) or an identical volume of DMSO (black circles) and imaged using a confocal microscope as a function of temperature. The percentage of phase separated GPMVs at each temperature was quantified using high content imaging software. Data are shown for one of two independent experiments for TLCK. Solid lines show Boltzmann fits to the data. ΔT_{misc} was defined as the difference in T_{misc} (i.e. the temperature at which 50% of the vesicles are phase separated) for the control and treated samples.

FIG. 10. TLCK and C6-ceramide have similar effects in GPMVs isolated from several different cell lines. GPMVs were isolated from the indicated cell types, labeled with DiD and NBD-DSPE, and treated with either DMSO or 10 μm of either TLCK or C6-ceramide for 1.5 h prior to imaging at RT. Data are from a single experiment performed in duplicate.

FIGS. 11A to 11F. Impact of TLCK, C6-ceramide and MβCD on lipid packing in GPMVs. (FIGS. 11A-11D) Representative fluorescence lifetime imaging microscopy (FLIM) images of GPMVs treated with DMSO (0.1%), TLCK (1 μM), C6-ceramide (1 μM), or MβCD (5 mM supplemented with 0.1% DMSO). Vertical color bar, Di4 lifetime color-bar scale (FIG. 11E) Average Di4 lifetime of GPMVs treated with DMSO, TLCK, C6-ceramide or MβCD. Data are presented as mean±SD for 56-170 GPMVs from 2 independent experiments. Each data point corresponds to average measurements for 5-15 GPMVs within a single lifetime image. P values were determined by unpaired one-way ANOVA with Sidak's multiple comparison test, Alpha=0.05 (95% confidence level), (****P<0.0001; n.s., not significant). Only non-phase separated GPMVs were included in the analysis for the DMSO, TLCK, and C6-ceramide-treated samples. Most MβCD-treated GPMVs contained multiple domains with same lifetime colors as shown in panel d which suggests uniform lipid packing. Thus, the average lifetime across all domains was measured for MβCD-treated GPMVs. (FIG. 11F) Average Di4 lifetime in Lo and Ld phases in DMSO-treated and TLCK-treated GPMVs. Data represent the mean±SD for a total of 25-30 GPMVs from 2 independent experiments. Each data point represents the average lifetime value obtained for 1-5 phase-separated GPMVs in a single image. P values were determined by unpaired one-way ANOVA with Sidak's multiple comparison test, Alpha=0.05 (95% confidence level) (****P<0.0001; n.s., not significant).

FIGS. 12A to 12C. Test of assay robustness based on SSMD* calculation. (FIG. 12A) Plate layout for SSMD* experiments. (FIG. 12B) Plot of % phase separated GPMVs versus well number in the order they were read on the plate. Data is from one representative experiment (n=3). Note the absence of systematic edge or well positional effects. (FIG. 12C) Table of SSMD* values for C6-ceramide and TLCK obtained from 3 independent replicates.

FIGS. 13A and 13B. Chemical structures of hits from the bioactivity lipid library that either increase (FIG. 13A) or decrease (FIG. 13B) the percentage of phase separated GPMVs.

FIGS. 14A and 14B. Chemical structures of hits from the bioactivity lipid library that either increase (FIG. 14A) or decrease (FIG. 14B) P_{ordered} for NBD-DSPE.

FIGS. 15A to 15F. Identification of phase separated GPMVs using the “colocalization” approach. (FIG. 15A) Example of a phase separated GPMV labeled with NBD-DSPE (green when shown in color) and DiD (magenta when shown in color). Arrow indicates angle=0°. Scale bar, 5 μm. (FIG. 15B) Plot of fluorescence intensity in the magenta (DiD) and green (NBD-DSPE) channels along the membrane contour as a function of angle for the vesicle shown in panel FIG. 15A. Note that in regions where green (NBD-DSPE) fluorescence is high, magenta (DiD) fluorescence is low, and vice versa. Solid lines correspond to membrane intensity and dotted lines represent background fluorescence. (FIG. 15C) Plot of the green (NBD-DSPE) fluorescence intensity versus magenta (DiD) fluorescence intensity at each position along the membrane contour. Populations of pixels that have high green fluorescence but low magenta fluorescence, versus high magenta fluorescence but low green fluorescence are observed below and above the diagonal (dotted line, shown as a blue dotted line), respectively. The presence of these two populations is indicative phase separation. Note that fluorescence intensities are normalized to the maximum measured values in each channel. (FIG. 15D) Example of a single phase GPMV labeled with NBD-DSPE (green when shown in color) and DiD (magenta when shown in color). Arrow indicates angle=0°. Scale bar, 5 μm. (FIG. 15E) Plot of fluorescence intensity in the magenta and green channels along the membrane contour (solid lines) as a function of angle for the GPMV shown in FIG. 15D. The periodic variations in fluorescence intensity in the magenta channel reflect polarization of DiD fluorescence. Background fluorescence is shown using dashed lines. (FIG. 15F) For a single-phase vesicle, the relative amounts of green and magenta fluorescence are similar for each position along the membrane contour and fall along the diagonal (dotted line, shown as a blue dotted line when shown in color). Fluorescence intensities are normalized to the maximum measured values in each channel. For consistency, GPMVs are identical to the ones shown in FIGS. 1A to 1J.

FIGS. 16A to 16D. Overview of approaches used to correct for positional effects and calculate Z-scores. (FIG. 16A) Representative example of positional effects. The percentage of phase separated vesicles per well is plotted as a function of the order in which they were imaged in a representative high throughput screen. Each datapoint (gray square) reports on the total number phase separated vesicles in a single well. Vertical dotted lines demarcate the beginning and end of each 96-well plate. The solid lines, shown as blue lines when in color (solid gray lines in the black and white figure) are the trendlines fit to the data for each plate. (FIG. 16B) Plot of the relative change in the percentage of phase separated vesicles per well, obtained by normalizing the data in panel a by the fitted trendline to correct for positional effects. (FIG. 16C) Data from plate 1 of FIG. 16B, replotted as a histogram. Outliers corresponding to wells with very high (far right bar, shown in magenta in color) or very low (far left bar, shown in green in color) percentage of phase separated vesicles are highlighted. (FIG. 16D) Representative plot of z-score as a function of well number for a single plate (plate 1 in FIG. 16B). Z-scores were calculated on a per-plate basis as described in the Materials and Methods. The dotted horizontal lines mark the position of Z=|2|. Compounds with a Z score >|2| are highlighted in magenta when shown in color, square near 8 on the y-axis and above about 40 on the x-axis, and green when shown in color, near −3 on the y-axis and above about 37.5 on the x-axis.

FIG. 17 is a File Folder Schematic depicting an exemplary location for the file VesA.m.

FIG. 18 is a depiction of an exemplary MATLAB menu bar as would be shown on an Apple Macintosh computer. In this example, the Run command button can be found under the EDITOR menu tab.

FIG. 19 is a depiction of a possible error message that can result from the first time VesA is launched in MATLAB showing the Change Folder option.

FIG. 20 is a depiction of a series of menu selections that relate to the steps that can be employed to analyze images. These include “Database”=>“Detect Vesicles”=>“Analyze Vesicles”=>“Results Vesicles”=>“Plates Summary. The final tab, “Configuration”, allows for saving or loading previously saved parameters for data analysis.

FIG. 21 depicts a window that, when starting VesA, pops up to permit selection of several different preset parameters. The “Demos” option loads preset parameters to analyze the demo images. The “HTS” option loads the settings used to analyze HTS data collected from 96-well plates using the Operetta microscope as described elsewhere herein. The “Temperature” option loads settings used to collect data from the temperature scans collected using a Zeiss 880 reported as described elsewhere herein. The “Free” option permits the user to freely define parameters used for data analysis.

FIG. 22 depicts an interaction with the “Database” tab. Using the “Database” tab, images can be loaded for analysis by selecting the “Load folder” (2a) or “Load files” (2b) buttons. The “Load plate” (2c) button can be used to load stacked images, but requires a setting in the configuration tab, in particular the definition of free parameters in the image filenames, by regular expressions. Once loaded, the image can be shown on the upper right hand corner of the window (2d). The user can select which image is shown, and what information is shown on the image using the check boxes directly underneath the image (2e) in the stack (plate) modus. In the standard modus the user can click on the smaller thumbnail images on the left (in demo 1 and 2 there is only one image). The numbers in the information box also depend on the method chosen in the “Results Vesicles” tab. For the demos they are fixed. Loading large images for the very first time may require some time. Since thumbnails are created during the first loading, subsequent loading of those images is typically much faster. If the image was analyzed in a former session, some statistical information is shown in the lower right corner of the window (2f). This was mainly used for software development purposes.

FIG. 23 depicts an interaction with the “Detect Vesicle” tab. On the “Detect Vesicle” tab, parameters used to detect the vesicles can be optimized and the contour/membrane intensity profiles can be visualized. First, vesicles need to be detected using the “Pre-Detect” button (3a). This performs a Hough transform of the superpositioned green and red channels. (https://www.mathworks.com/help/images/ref/imfindcircles.html). Rₘᵢₙ and Rₘₐₓ (units of pixels) must be set manually according to the size of the vesicles in the image. The “auto” setting for sensitivity works for well in many instances and can be used in initial testing. This parameter can also be manually adjusted if desired, for example if not enough vesicles are detected. The “Pre-Selection” function (3b) performs a Hough transform of the green channel and red channels individually. Using the dropdown menu and checkboxes, this step permits the user to define whether the vesicles to be analyzed are required to contain signal in both the green and red channel (“all”) or any channel, i.e. green or red (“any”). Pre-selection is not necessary for initial, simple tests, and can be skipped by selecting “none” in the dropdown menu under “needed channels”. Next, the membrane/contours need to be identified (3c). As a first step, the “maximum” method should be selected, since it is much faster than the “fit” method. “/1/2 . . . ” indicate how many points/angles are skipped doing the detection, and are only important for the “fit” method. Left clicking on a pre-detected vesicle in the large image starts the contour detection process. In addition to thumbnail image of the chosen vesicle (3d), the results of the contour detection can be shown graphically for each channel (3e). “Background distance” (3f) defines the distance from each vesicle at which background should be measured. The distance is defined either as terms of absolute number of pixels or as a percentage of the vesicle radius. Background can be measured locally for each vesicle, or globally as a mean value. “Filter” (3g) can be used to perform two-dimensional image Gaussian filtering. The “line filter” option is applied to the contour (angular) and the radial intensities to find the contour position (radial). “Constraints” (3h) can be applied to narrow the population of vesicles to be analyzed. This process is important for analyzing whole images or sets of images but not for single vesicles. Following the predetection and preselection steps, the identified objects can be visualized in the image (3i) by choosing the corresponding checkbox (3j). The total number of vesicles detected and those remaining after preselection are shown in the bottom right panel (3k).

FIG. 24 depicts an interaction with the “Analyze Vesicle” tab. On the “Analyze vesicle tab,” individual vesicles can be fully analyzed by left clicking on the vesicle in the large image on the right. The selected vesicle is shown in the thumbnail image. Results obtained from three different analysis methods (threshold, distribution, and colocalization) are shown for the chosen vesicle. They include graphical depictions of the fluorescence intensities in Ch1 and Ch2 for the chosen vesicle for the “threshold” method (4a, 4b, 4c), “distribution” method (4d, 4e) and “colocalization” method (4f, 4g). If desired, these graphs can be saved for individual vesicles. Note that only results obtained using the “distribution” and “colocalization” methods are used for data analysis elsewhere herein. Also note that even though the thumbnail image updates for all GPMVs when they are clicked on, the graphs are only updated for GPMVs that pass all exclusion criteria that are selected. The calculated partition coefficients (±SD) are also reported (4h). Ch1/Ch1 reports the partition coefficient for the fluorophore in Ch1 in the phase where it is brightest, i.e. Ch1/Ch1 reports the partition coefficient of fluorophore in Ch1 relative to the position of the enriched phase defined by Ch1. Ch2/Ch1 reports the partition coefficient for Ch1 in the phase where Ch2 is brightest (i.e. the opposite phase). The percentage of the vesicle membrane (±SD) that is enriched in red fluorescence (Ch1) or green fluorescence (Ch2) is also provided (4i). An opportunity to save a summary of the numerical results in 4h and 4i for all of the vesicles in the image is provided on the next tab (“Results vesicles”). The table in the lower right is automatically be updated with results for the vesicle population (4j). It is not necessary to click the (Re)run button.

FIG. 25 is a depiction of an interaction with the “Results Vesicles” tab. In the “Results Vesicles” tab, a single image, “well” (stack of images) or “plate” (set of image stacks) can be automatically analyzed by clicking on “(Re) analyze” button (5a) after selecting the associated list entry (5b). Since all data is saved during the analysis of an image, it is not necessary to repeat the detection, preselection, and analysis steps from the previous tabs. Statistical analysis for the entire population of selected vesicles can be obtained by clicking on “(Re)Sum” button (5c) after choosing the associated list entry (5d), methods (5e) and channels of interest (5f). Graphs showing the results are then shown. In this example, graphs showing the partition coefficients (5g) and percent phase separated vesicles (5h) are shown together with a plot of the size distribution of selected vesicles (5i). Results of the analysis can be saved as images or output the data in table format (5j). It is also possible to output a table that provides the results for individual vesicles on a vesicle-by-vesicle basis by selecting “Say sel vesicles” (5k).

FIG. 26 is a depiction of an interaction with the “Plate Summary” tab. The Plate Summary tab collates and statistically analyzes sets of images stacks (for example as obtained for multiwell plates). This information can be combined with external information (such as concentration, temperature, drug treatment, etc.) in table format. Because this step is designed to collate data across multiple stacks of images, a single image or single image stack can be represented as a single data point when displayed on this tab.

FIG. 27 is a depiction of an interaction with the “Configuration” tab. The Configuration tab can be used to set internal parameters used by VesA, such as the patterns to identify external parameters in the image filenames and the cross-talk matrix for the channels. Individual components are as follows:
Save/load settings. The “Save Settings” [72] and “Load Settings” [73] buttons can be used to save or load all values in the graphical interface. It might be useful to save the settings for a full analysis for each run. VesA also retains all settings when the program is closed and restores them when it is started again.
“Collate Files/Wells” frame—filename patterns. In the “Parameter(s)” box [63] a filename pattern can be set that identifies up to two free parameters used to identify a “well” belongings. Here, the free parameters are defined in curly brackets { }. The patterns are in the style of regular expressions: Depending on whether the parameter are capital letters, small letters or numbers the notations are {A}, {a} or {0} with any repetitions, for example a three-digit number would be defined as {000}, or a combination of capital letters would be {AA}. A mix of numbers and letters as a parameter is not currently allowed but its implementation is ongoing. Also, the use of underscores or decimal points are not currently supported but is also under development. The number of digits or letters defining the parameter (not the filename itself) must be the same for all images, although a variable length of numbers is also under development. An asterisk “*” represents any expression with a variable length. In case there is no asterisk at the beginning or end of the pattern, the filename must begin, or end respectively, with the given expression.
The “Combine Channels” box [64]. A pattern for filenames to combine channel separated image files can be defined. It follows similar rules as for the “Parameter(s)” patterns.
The “Plate name pattern” box [65]. An extraction of a certain plate name pattern of the folder containing the plate/images can be defined. Next to that box preview can be shown after the “test” button [68] is used. No asterisks “*” are allowed, but their inclusion is under development. The content in the curly brackets { } define the plate name. It can be any combination of capital letters “A”, small letters “a”, numbers with of defined digits “0” or numbers with a variable length “d”.
If the box is left blank, or the pattern could not be found in the folder name the whole folder name is considered as a plate name. The “Image folder” [66] and “Result folder” [67] subfolders can be defined where VesA is looking for images and saves all internal tables, respectively. In case the result folder does not exist, it is generated automatically. Both boxes can be kept empty. In that case VesA will not look for image subfolders and saves the tables aside the image files.

FIGS. 28A and 28B. Results from library of known drugs (1184) screen for compounds that alter the phase domain preference of PMP22 in GPMVs. The X axis is the same for both plots, but the Y axis gives the impact of each compound on the relative populations of the ordered (raft) phase vs. the disordered phase as determined directly in the PMP22 screen (FIG. 28A) versus in a different screen of the same compounds conducted using GPMVs with no PMP22 present (FIG. 28B). X'd points represent cases where the results should be regarded as false positives. Crystal violet is also a false positive, but via a different mechanism than the other false positive cases. Three compounds are labeled on the right of each plot that we believe are true and statistically robust hits that increase the PMP22 population in the ordered (raft) phase from the control value of 78% by about 10% in each case (see the EXAMPLES below for additional information).

### DETAILED DESCRIPTION

In some embodiments, the presently disclosed subject matter provides a high-throughput screening method for identifying molecules/compounds that impact the localization of membrane proteins to lipid rafts (which can impact their functions in the case of some membrane proteins).

Many membrane proteins preferentially associate with cholesterol and sphingomyelin-rich bilayer nanodomains known as lipid rafts, leading to altered functionality. Despite the importance of protein association with lipid rafts in many diseases, pharmacological tools for directly and specifically inhibiting or enhancing raft association of proteins do not exist. An aspect of the presently disclosed subject matter was to discover small molecules that alter partitioning of membrane proteins between raft and fluid domains by (i) developing tools for high throughput screening and (ii) discovering and characterizing first-in-class small molecule probes that alter the partitioning of membrane proteins between raft-like nanodomains and the surrounding fluid phase membrane. In accordance with some aspects, the presently disclosed subject matter provides a novel pharmacological paradigm for modulating protein function that can lead to a completely new class of therapeutics. The approach outlined here can also identify small molecules that can be used to modulate rafts themselves by either stabilizing or disrupting their structure. Such compounds are of great practical value, given that few chemical methods to manipulate rafts in a controlled manner currently exist.

In representative, non-limiting embodiments, using Giant Plasma-Membrane Derived Vesicles (GPMVs) as a model system, two assays were developed for high throughput screening of small molecule libraries. The first assay identified compounds that modulate membrane phase behavior by disrupting or stabilizing rafts. The second screens for compounds that alter the partitioning of proteins between raft-like nanodomains and the surrounding fluid phase. In some embodiments, software and analytical methods are provided to facilitate high throughput identification of GPMVs, to determine the extent of lipid phase separation, and to quantify membrane protein partitioning, such as between lipid raft and non-raft phase domains. While this software was developed to facilitate analysis of HTS data, it can be used more generally for the analysis of GPMVs as well as other model membrane systems such as giant unilamellar vesicles. Representative source code for the software is provided herein below.

### I. GENERAL CONSIDERATIONS

Major barriers to the discovery of small molecule modulators of rafts is that in cells, rafts are diffraction-limited in size and are only transiently stable (Levental et al., 2020a). Thus, raft modulators need to be identified in model systems that allow for the visualization of large-scale domains while also retaining the complexity of cell membranes. Giant Plasma Membrane Vesicles (GPMVs) offer an attractive solution to this problem (Baumgart et al., 2007; Sezgin et al., 2012a; Levental & Levental, 2015; Gerstle et al., 2018). Derived from the plasma membrane of adherent cells, these micron-scale blebs comprise a complex mixture of proteins and lipids reflective of the parent cell plasma membrane composition. A key feature of GPMVs is their ability to undergo temperature-dependent demixing that leads to the formation of coexisting macroscopic liquid raft-like and non-raft domains. These domains share features with liquid-ordered and liquid-disordered phases found in synthetic model membranes and can be labeled with phase-specific fluorescent dyes, allowing for their visualization using standard fluorescence microscopy approaches (Baumgart et al., 2007; Sezgin et al., 2012a; Levental & Levental, 2015; Gerstle et al., 2018). The temperature at which 50% of GPMVs are phase separated under a given set of experimental conditions is defined as the miscibility transition temperature, T_{misc}. T_{misc} varies as a function of cell type and growth conditions and is hypothesized to reflect the size and lifetime of nanoscopic domains at physiological temperature (Veatch et al., 2008). Changes in T_{misc} have also been correlated to lipid order as measured by other analytical methods (Levental et al., 2011; Podkalicka et al., 2015; Cornell et al., 2017). Thus, T_{misc} provides a simple and robust measure of raft stability (Miller et al., 2020). T_{misc} is also sensitive to the effect of small molecules and bioactive compounds (Gray et al., 2013; Zhou et al., 2013; Raghunathan et al., 2015; Machta et al., 2016; Cornell et al., 2017; Gerstle et al., 2018). Herein, this behavior is exploited to develop an unbiased, high-throughput screen (HTS) using GPMVs to identify new chemical modulators of rafts.

Membrane proteins constitute the largest fraction of druggable targets with a majority of drugs being developed to alter their function. Most drugs bind to proteins in ways that affect their functions. The function of membrane proteins also depends on the environment in which they reside. A number of membrane proteins and receptors are thought to localize within specialized cholesterol-rich membrane phases called lipid rafts. Thus, rafts represent a potentially druggable target. However, identification of small molecules that target rafts is still a nascent field and only a small number of compounds have been described in the literature with this activity. Therefore, in accordance with embodiments of the presently disclosed subject matter, a platform is developed and provided to perform unbiased, high-throughput screens to identify small molecules that can alter membrane physical properties, in particular lipid rafts. To do so, a computational approach was also developed to quantify small molecule-induced changes in plasma membrane phase behavior using giant plasma membrane vesicles (GPMVs) as a model system. The effect of thousands of small chemical compounds, including a collection of 850 bio-active lipids, on membrane phase behavior of GPMVs was determined. These screens lead to the identification of several known raft modulators as well as several new ones. These candidates were validated by transition temperature-perturbation measurements. Thus, by quantifying the impact of thousands of different compounds on membrane phase behavior, an approach to discover new tools to pharmacologically modulate lipid rafts is provided.

Many membrane proteins preferentially associate with cholesterol and sphingomyelin-rich bilayer nanodomains known as lipid rafts, leading to altered functionality. Despite the importance of protein association with lipid rafts in many diseases, pharmacological tools for directly and specifically inhibiting or enhancing raft association of proteins do not exist. An aspect of the presently disclosed subject matter was to discover small molecules that alter partitioning of membrane proteins between raft and fluid domains by (i) developing enabling tools for high throughput screening and (ii) discovering and characterizing first-in-class small molecule probes that alter the partitioning of membrane proteins between raft-like nanodomains and the surrounding fluid phase membrane. The presently disclosed subject matter establishes a novel pharmacological paradigm for modulating protein function that may lead to a completely new class of therapeutics. The presently disclosed subject matter can also identify small molecules that can be used to modulate rafts themselves by either stabilizing or disrupting their structure. Such compounds would also be of great practical value, given that few chemical methods to manipulate rafts in a controlled manner currently exist.

### II. DEFINITIONS

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes. Biological samples can also include cells from any organism that are transfected with a gene that encodes a particular protein in either its native sequence or an engineered sequence, the latter including forms of the protein that include either a fused fluorescent protein or an added epitope.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

As used herein, the term “biologically active fragments” or “bioactive fragment” of the polypeptides encompasses natural or synthetic portions of the full-length protein that are capable of specific binding to their natural ligand or of performing the function of the protein.

### III. REPRESENTATIVE METHODS, SYSTEMS, AND COMPUTER READABLE MEDIA

In some embodiments, the presently disclosed subject matter provides approaches using Giant Plasma-Membrane Derived Vesicles (GPMVs) as a model system. Particularly, in representative, non-limiting embodiments using GPMVs, two assays were developed for high throughput screening of small molecule libraries. The first assay identified compounds that modulate membrane phase behavior by disrupting or stabilizing rafts. The second screens for compounds that alter the partitioning of proteins between raft-like nanodomains and the surrounding fluid phase. Also provided in some embodiments are software and analytical methods to facilitate high throughput identification of GPMVs, determine the extent of lipid phase separation, and quantify membrane protein behavior, such as partitioning, such as between lipid raft and non-raft phase domains. While this software was developed to facilitate analysis of HTS data, it can be used more generally for the analysis of GPMVs or related model systems composed of membrane vesicles such as giant unilamellar vesicles.

In a particular representative embodiment, the raft affinities of several disease-related membrane proteins were tested in order to identify potential candidates for the screen. It was found that human peripheral myelin membrane protein 22 (PMP22), which causes most cases of the common peripheral neuropathy Charcot-Marie-Tooth disease, preferentially partitions into the raft domains of GPMVs. While PMP22 to the knowledge of the present co-inventors is the first example of a multi-pass transmembrane protein shown to have affinity for raft domains in GPMVs (Justin T. Marinko et al. (2020) Proceedings of the National Academy of Sciences (USA) 117, 14168-14177), several other examples of multi-pass transmembrane proteins that also show a preference for residing in raft domains were subsequently identified by others (Castello-Serrano, et al., (2020) J Phys Chem B 124, 5930-5939). In addition, the screening approach is not limited to multi-pass transmembrane proteins; it can essentially be applied to any membrane protein, including single pass transmembrane proteins, peripheral membrane proteins, and proteins that bind to protein or lipid receptors in the membrane, such as cholera toxin B-subunit.

The HTS assay was also used to screen the effects of a library of bioactive lipid libraries and FDA-approved drugs on raft formation in GPMV at the High Throughput Screening Facility of Vanderbilt University, Nashville, Tennessee, United States of America. Preliminary screens were also performed using NIH clinical set I and II, NCI mechanism set and Spectrum collection libraries. The bioactive lipid library and FDA approved drug library screen were repeated multiple times and yielded a number of hits, including both raft disruptors and raft stabilizers. A subset of these compounds were validated by showing they behave in a dose-dependent manner. These findings suggest already existing drugs could be exploited as raft modulators both in the laboratory and in the clinic.

HTS was performed to identify FDA-approved drugs that either reduce or further enhance the raft phase partitioning of PMP22. Analysis of these datasets is described in the Examples below. Hits have been observed for compounds that alter the phase partitioning of PMP22 and that exhibit other activities that can be detected by the presently disclosed assay methods, which can include imaging assays.

The presently disclosed subject matter provides in some embodiments a high content imaging-based screen to identify new modulators of membrane phase behavior and membrane protein behavior. To do so the process of identifying phase separated vesicles was automated in a high-throughput manner. A pilot screen of a bioactive lipid library uncovered examples of both known and new raft modulators. The automated image analysis can be accomplished using software as disclosed herein. This software also measures other parameters of interest such as partition coefficients, fraction of membrane phases, size distributions, membrane intensities, intra- and extra-vesicular intensities. Further chemical libraries, such as the FDA library, have also been screened and are currently being validated.

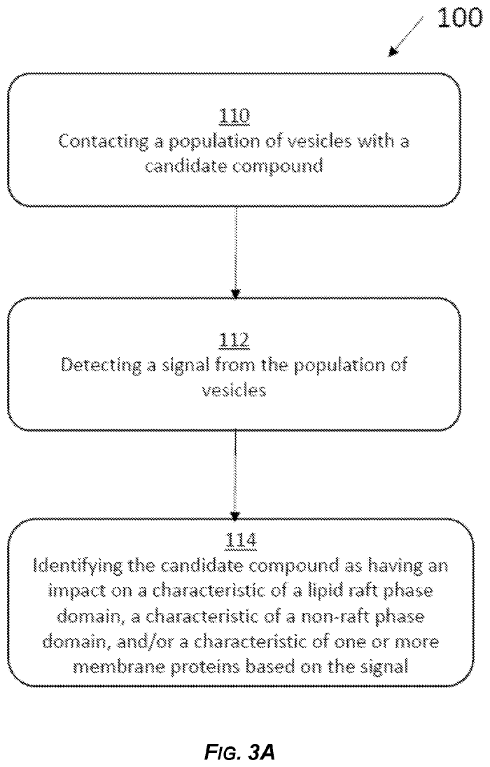

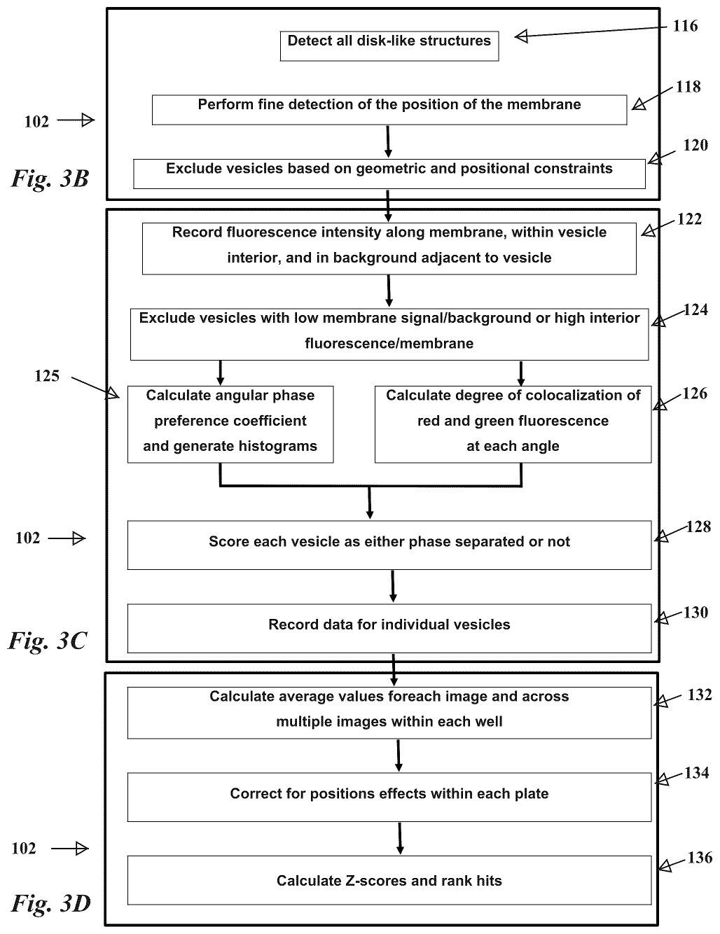

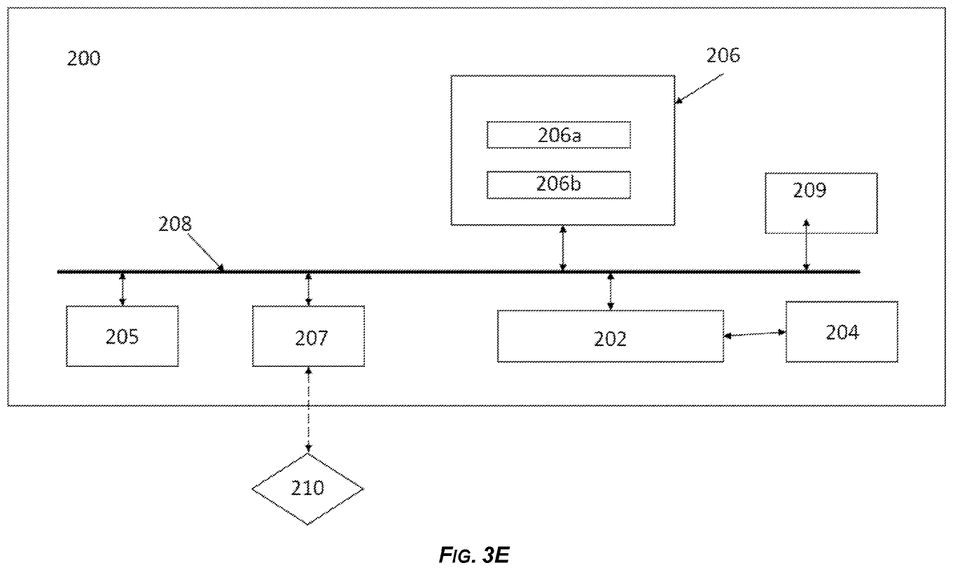

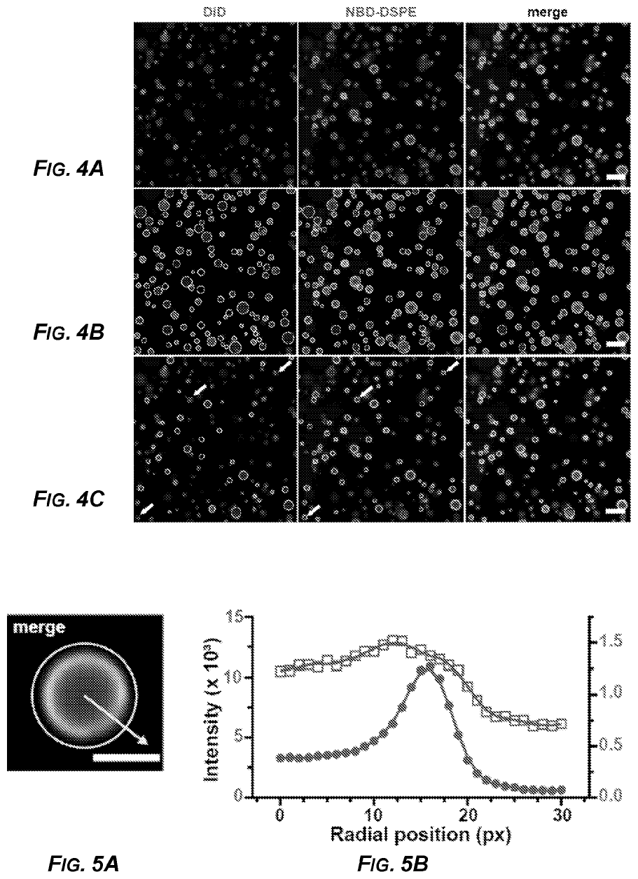

In some embodiments, the presently disclosed method for identifying a compound that impacts a characteristic of a lipid raft phase domain, a characteristic of a non-raft phase domain, and/or a characteristic of one or more membrane proteins can comprise method 100 of FIG. 3A, which comprises step 110 of contacting a population of vesicles with a candidate compound. In some embodiments, the population comprises a mixture of some vesicles which comprise a membrane comprising a lipid raft phase domain and a non-raft phase domain, and some vesicles which comprise a membrane comprising a single phase. In some embodiments, the vesicles which comprise a membrane comprising a lipid raft phase domain and a non-raft phase domain, one or both of which further comprise one or more membrane proteins, and/or the vesicles which comprise a membrane comprising a single phase further comprises one or more membrane proteins. By way of elaboration and not limiting, the presently disclosed subject matter comprises contacting a population of vesicles, wherein one or more vesicles in the population of vesicles optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are detectable and distinct from one another, referred to as phase separated; detecting a signal from the population of vesicles; and identifying the candidate compound as having an impact on a characteristic of a lipid raft phase domain, a characteristic of a non-raft phase domain, and/or a characteristic of one or more membrane proteins based on the signal. Steps of method 100 and indeed any steps/methods/processes/algorithms disclosed herein can be embodied by a module, which can be stored in memory and executed by a hardware processor.

Continuing with reference to FIG. 3A, method 100 can comprise step 112 of detecting a signal from the population of vesicles, such as by imaging the population of vesicles. In some embodiments, step 112 comprises, for one or more vesicles in the population of vesicles, fluorescence microscopic imaging. Continuing with FIG. 3A, method 100 can comprise step 114 of identifying the candidate compound as having an impact on a characteristic of a lipid raft phase domain, a characteristic of a non-raft phase domain, and/or a characteristic of one or more membrane proteins based on the signal. In some embodiments, step 114 comprises assessing whether the candidate compound induces changes in a percent of vesicles containing phase separated lipid raft phase and non-raft phase domains within the population of vesicles, and/or changes in the relative sizes and/or populations of the lipid raft phase domain versus the non-raft phase domain, and/or the distribution of at least one of the one or more membrane proteins, in some embodiments, the distribution of at least one of the one or more membrane proteins between the lipid raft phase and the non-raft phase domains.

In some embodiments, the signal is used to assess whether the candidate compound induces changes in the percent of vesicles containing the single visible phase as compared to the lipid raft phase and non-raft phase domains within the population of vesicles; changes in the relative sizes and/or characteristics of the lipid raft phase domain versus the non-raft phase domain; and/or changes or lack thereof in the distribution of one or more membrane proteins between the lipid raft phase domain and the non-raft phase domain. In some embodiments, the characteristic of a lipid raft phase domain, a characteristic of a non-raft phase domain, and/or a characteristic of one or more membrane proteins comprises membrane phase behavior, partitioning of proteins, such as between the lipid raft phase domain and the non-raft phase domain, or both.

Any suitable vesicle as would be apparent to one of ordinary skill in the art upon a review of the instant disclosure can be employed in accordance with the presently disclosed subject matter. In some embodiments, the vesicle comprises a Giant Plasma-Membrane Derived Vesicle (GPMV). GPMVs, can be made by two different methods, the NEM method and the DTT method. The resulting GPMVs have somewhat different properties. In some embodiments, the presently disclosed subject matter used GPMVs prepared using the DTT method. This is because the design of the assay involves that the population of GPMVs contain a mixture of phase separated and non-phase separated vesicles at room temperature, so that changes in the percentage of phase separated vesicles in response to the presence of the small molecules can be detected. For GPMVs prepared using the other method (the NEM method), at room temperature, essentially none of the vesicles are phase separated. For the NEM method, the assay should be performed at a lower temperature (such as 4 degrees Celsius) to obtain a population of vesicles that comprises a mixture of phase separated and non-phase separated vesicles.

With regard to selection of vesicles, in some embodiments, the population of vesicles is selected so that it comprises a mixture of vesicles where i) there is only a single visible phase and ii) a population that are measurably “phase separated,” meaning that both the raft and non-raft domains are visible at a given temperature or other parameter. In principle, any given vesicle can transition between a single phase and becoming phase separated in response to a number of parameters, such as temperature. In some embodiments, the presently disclosed subject matter seeks to identify compounds that make it easier or harder for the vesicles to make this transition at a single temperature. This can be read out, for example, by looking for changes in the percentage of phase separated vesicles in the presence and absence of the compounds. Also, whether compounds cause changes in the relative sizes of the domains can be detected. That is, the term “relative size” refers to how much lipid raft phase domain versus non-raft phase domain is present in a given vesicle.

In some embodiments, one or more membrane proteins is labeled with one or more fluorescent labels, each of which could be a fused fluorescent protein, a chemically-reacted fluorescent compounds, or an unnatural amino acid that either is fluorescent or can be reacted with a fluorescent compound. The one or more proteins are then detected, such as with fluorescence microscopy, and quantitated in each phase using the software. By way of example, PMP22 containing a MYC epitope sequence can be employed in a vesicle such as a GPMV, such as by expressing a MYC-Tagged PMP22 in Hela cells, followed by use of a commercial fluorescently labeled anti-MYC antibody to confer detectable fluorescence to PMP22 in GPMVs. Indeed, the fluorescent labels come in various colors, which means that one can detect several differently labeled molecules at the same time in the assay, such as by using a microscope. In some embodiments, for example, where one uses a label that marks the non-raft phase domain in red, one can label a membrane protein (like PMP22) in green. By visualizing them using fluorescence microscopy, one can then detect whether the PMP22 is in the lipid raft phase or non-raft phase domain, and to what extent. By way of a particular example, PMP22 prefers to localize within the raft phase domain, as evidenced by a finding that it is mostly not in the non-raft phase domain. There are some proteins, on the other hand, that exhibit a preference not to reside in rafts, such as a protein known as C99. (Capone et al., 2021). If one labels the C99 green and again uses a red non-raft marker, in that case both colors overlap in the same phase. Note that the convention here is that when they overlap they appear yellow in the merged image. In some embodiments, the presently disclosed methods and systems seek to identify compounds that change the “affinity” of a particular protein for the raft vs. non-raft phase domain. In some embodiments, this is read out as a change in relative brightness of the fluorescence signal associated with that protein in one phase versus the other.

Other types of model membrane systems that can be employed in the presently disclosed subject matter in addition to GPMVs include, but are not limited to: giant unilamellar vesicles with membrane compositions tuned to form liquid ordered (raft) and liquid disordered (non-raft) domains (Veatch & Keller, 2003; Wesolowska et al., 2009; Sych et al., 2021); plasma membrane spheres (Lingwood et al., 2008); endoplasmic reticulum-derived vesicles capable of undergoing phase separation (King et al., 2020), plasma membrane vesicles in which phase separation is mediated with the help of membrane-associated condensates (Zhao & Zhang, 2020); and yeast vacuoles, which exhibit phase separation under physiological conditions (Toulmay & Prinz, 2013; Rayermann et al., 2017).

Examples of specific properties of raft and non-raft domains, e.g. characteristics, that could be examined using this general approach beyond their size and propensity to undergo phase separation include:

Fluidity and order. This can be assessed using a range of reporters that can be imaged using fluorescence or related microscopy approaches (Kaiser et al., 2009; Sezgin et al., 2015; Steinkuhler et al., 2019; Sych et al., 2021).

Partitioning, accessibility, or conformation of specific lipid species in raft and non-raft domains. This can be accomplished, for example, using fluorescently labeled lipids or fluorescent lipid analogs (Gimpl & Gehrig-Burger, 2011; Chinnapen et al., 2012; Sezgin et al., 2016) or proteins that can selectively bind to specific lipids and/or recognize specific lipid complexes or conformations (Raghunathan et al., 2016; Johnson et al., 2017; Endapally et al., 2019).

Chemical properties of lipids. As one example, levels of lipid peroxidation, a process that occur in response to oxidative stress, can be monitored using fluorescent reporters (Drummen et al., 2002; Tsubone et al., 2019).

In some embodiments, the presently disclosed subject matter optimizes the parameters used in the high content image analysis software for different experimental set-ups and to visually inspect images during this process. While the image analysis process is automated using the software disclosed herein, user input can be employed to define and optimize the parameters to be used, as would be apparent to one of ordinary skill in the art upon a review of the instant disclosure. Automated data analysis is “tuned” to optimize the process for the specific format of the datasets being input. For example, florescence microscopic images can be collected at different magnifications, resulting in images that contain GPMVs that differ in size due to differences in magnification. This would involve one using different settings for the upper and lower limit of the size of circular objects that the software looks for as part of the vesicle detection.

Additionally, as would be apparent to one of ordinary skill in the art upon a review of the instant disclosure, a user should desirably visually inspect the data as part of the process of setting up the automated data analysis pipeline to make sure that the software is detecting the GPMVs appropriately and that the GPMV preparation looks normal. It can also be helpful to look at the images after defining the hits, since some compounds might change the size or shape of the GPMVs, or have other unexpected effects.

As would be appreciated by one of ordinary skill in the art upon a review of the instant disclosure, the presently disclosed GPMV system provides a very effective model system to identify small molecules that modulate rafts. Thus, certain aspects of a GPMV system, such as having a lipid composition similar to plasma membrane but exhibiting a partial loss of lipid asymmetry (Sezgin et al., 2012a), lacking an underlying actin cytoskeleton, which normally functions to corral membrane domains and sustain or dampen lipid-mediated heterogeneity (Chichili & Rodgers, 2009; Honigmann et al., 2014; Lietha & Izard, 2020); and differing membrane curvature between GPMVs and live cell membranes; do not preclude the effective use of GPMVs in the presently disclosed subject matter. Indeed, recognition of these aspects provides guidance to one of ordinary skill in the art in employing a vesicle in an assay of the presently disclosed subject matter.

The hits resulting from the assay can be validated in multiple biological systems to confirm that they function as raft modulators, as predicted from the GPMV results. For example, it can be determined whether the compounds identified here modulate raft-mediated functions in cells such as clathrin-independent endocytosis or immune signaling (Stone et al., 2017; Verma et al., 2018). It is possible that some may have activities in GPMVs but not in cells, for a variety of reasons. However, this does not undermine the value of identifying small molecule have an impact on rafts or proteins associated with rafts as disclosed herein. For example, establishing whether their raft-modulating activities in GPMVs are predictive—or not—of their biological consequences should help lead to a better understanding of the limitations of the raft hypothesis itself.

In some embodiments, the lipid raft phase domain, the non-raft phase domain, and/or one or more membrane proteins are distinguishably labeled. In some embodiments, (a) the lipid raft phase domain is labeled with a first fluorescent label and the non-raft phase domain is labeled with a second fluorescent label, wherein the first fluorescent label and the second fluorescent label are distinguishable from each other; (b) the lipid raft phase domain is labeled with a first fluorescent label and the one or more membrane proteins is labeled with a second or more fluorescent label, wherein the first fluorescent label and the additional fluorescent label(s) are distinguishable from each other; (c) the non-raft phase domain is labeled with a first fluorescent label and the one or more membrane proteins is labeled with a second or more fluorescent label, wherein the first fluorescent label and the additional fluorescent label are all distinguishable from each other; and (d) the lipid raft phase domain is labeled with a first fluorescent label, the non-raft phase domain is labeled with a second fluorescent label, and the one or more membrane proteins is labeled with a third or more fluorescent label, wherein the first fluorescent label, the second fluorescent label, and the third or more fluorescent label are distinguishable from each other.

Representative fluorescent labels are disclosed herein, but any suitable fluorescent label as would be apparent to one of ordinary skill in the art upon a review of the instant disclosure is provided in accordance with the presently disclosed subject matter. A comprehensive list of fluorescent lipid probes defined as markers of raft or non-raft domains in the plasma membrane based on their partitioning into two coexisting liquid domains (raft and non-raft) in giant unilamellar vesicles and GPMVs, together with the original references, can be found in Table 1 of Kusumi et al., (2020) Traffic 21, 106-137. Selected, non-limiting examples include:
Raft (liquid ordered domain phase) markers in GPMVs
488neg-SM(18:0) (Hd)
594neg-DSPC (Hd)
594neg-SM(18:0) (Hd)
GM1+Cholera toxin B-subunit
NBD-DPPE
Non-raft (liquid disordered domain phase) markers in GPMVs
LRB-DOPE
All the phospholipid probes, DiI probes, and DiO probes containing two oleoyl or linoleoyl chains, including ATTO647N-DOPE, ATTO594-DOPE, DiI-C18:1, DiI-C18:2 (FastDiI), DiO-C18:3 [Fast DiO]
DiI-C16:0
TexasRed-DPPE

Referring now to FIGS. 3B and 3C, in some embodiments of a method of the presently disclosed subject matter, referred to as 102, detecting the signal, such as by imaging, can comprise identifying one or more vesicles such as by detecting all disk-like structures 116; performing fine detection of a position of a membrane of a vesicle in the population of vesicles 118, such as by mapping a position of membrane contour for each of the one or more vesicles; excluding one or more vesicles that do not meet one or more selection criteria 120, such as geometric and/or positional constraints; recording fluorescence intensity along the membrane, within the vesicle interior, and in background adjacent to the vesicle 122, such as by generating a plot of fluorescence intensity along the membrane of the vesicle; excluding vesicles with low membrane signal/background or high interior fluorescence/membrane 124; calculating a partition coefficient of a vesicle, such as by calculating angular phase preference coefficient and generating histograms 125, calculating a degree of colocalization of fluorescence, such as red and green fluorescence at each angle 126; scoring a vesicle as phase separated or not 128, or any combination of any of the foregoing. Method 102 can comprise recording data for individual vesicles 130; calculating average values for each image and across multiple images within each well 132; correcting positional effects within each plate 134; and calculating Z-scores and ranking hits 136. Representative, non-limit exclusion criteria for vesicles include but are not limited to non-circular, only labeled with a single dye, out of focus (a primary reason), too close to each other, low signal to noise, strong drifting, membrane intensity too high, and interior intensity too high. Steps of method 102 and indeed any steps/methods/processes/algorithms disclosed herein can be embodied by a module, which can be stored in memory and executed by a hardware processor.

In some embodiments, calculating a partition coefficient of a vesicle comprises calculating the partition coefficients of the fluorescent labels and/or fluorescently labeled membrane proteins as a function of angle around the entire vesicle. In some embodiments, scoring a vesicle as phase separated or not comprises determining whether a histogram of the angular partition coefficient of a vesicle comprises a single peak centered around 0.5 or instead contains multiple peaks reflecting a presence of fluorophore-rich and fluorophore-poor domains. In some embodiments, scoring a vesicle as phase separated or not comprises separating data points into two separate quadrants for vesicles having phase separated membranes and colocalizing datapoints on a diagonal for membranes containing a single uniform phase.

In some embodiments, e.g. methods 100 (FIG. 3A) and/or 102 (FIGS. 3B-3C), identifying the candidate compound as having an impact on a characteristic of a lipid raft based on the signal comprises calculating Z-scores and ranking hits, e.g. 136 in FIG. 3D; detecting a change in phase separation in one or more vesicles in the population of vesicles between the lipid raft phase domain and the non-raft phase domain, e.g. 128 in FIG. 3C, and/or detecting a change in at least one of the one or more membrane proteins from the lipid raft phase domain or the non-raft phase domain to the other phase. In some embodiments, detecting a change in phase separation comprises detecting a change in partition coefficients and/or a change in percent of phase separated vesicles in the population of vesicles. In some embodiments, the measured partition coefficient is for the distribution of a fluorescently-labeled protein between lipid raft and non-raft phase domains.

Referring now to FIG. 3E, a system for identifying the candidate compound as having an impact on a characteristic of a lipid raft phase domain, a characteristic of a non-raft phase domain, and/or a characteristic of one or more membrane proteins is referred to generally at 200. In some embodiments, the system is configured to function automatedly. In some embodiments, system 200 comprises a plate 204 comprising a plurality of reaction wells for contacting a population of vesicles with a candidate compound, wherein one or more vesicles in the population of vesicles comprises a membrane comprising a lipid raft phase domain and a non-raft phase domain and optionally one or more membrane proteins, wherein in a portion of the population of vesicles there is only a single detectable, e.g. visible phase, and in a portion of the population of vesicles both the raft and non-raft domains are visible. By way of elaboration and not limitation, the presently disclosed subject matter comprises contacting a population of vesicles, wherein one or more vesicles in the population of vesicles optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are detectable and distinct from one another, referred to as phase separated. In some embodiments system 200 comprises a computing platform including a processor and memory, the computing platform including a module configured to detect a signal from the population of vesicles using a microscope, camera or a sensor communicatively coupled to the computing platform; and a module to identify the candidate compound as having an impact on a characteristic of a lipid raft phase domain, a characteristic of a non-raft phase domain, and/or a characteristic of one or more membrane proteins based on the signal.

Continuing with reference to FIG. 3E, system 200 can be any suitable entity (e.g., a mobile device, a server, or any other computing device) configurable to detect a signal from the population of vesicles using a microscope, camera or a sensor communicatively coupled to the computing platform; and to identify the candidate compound as having an impact on a characteristic of a lipid raft phase domain, a characteristic of a non-raft phase domain, and/or a characteristic of one or more membrane proteins based on the signal. System 200 can thus comprise processor(s) 202. Processor(s) 202 can represent any suitable entity or entities (e.g., hardware-based processor) for processing information and executing instructions or operations. Processor(s) 202 can be any type of processor, such as a central processor unit (CPU), a microprocessor, a multi-core processor, and the like. System 200 can further include memory 206 for storing information and instructions to be executed by processor(s) 202.

In some embodiments, memory 206 can comprise one or more of random access memory (RAM), read only memory (ROM), static storage such as a magnetic or optical disk, or any other type of machine or non-transitory computer readable medium. In some embodiments, memory 206 can store first module 206a and second module 206b for instructions described hereinabove. Notably, each of first module 206a and second module 206b can be executed by a processor when stored in memory 206. In some embodiments, memory 206 can store data from a sample data set.

As further illustrated in FIG. 3E, system 200 can further include user interface 205, e.g., for displaying and/or transferring to detect a signal from the population of vesicles using a microscope, camera or a sensor communicatively coupled to the computing platform; and to identify the candidate compound as having an impact on a characteristic of a lipid raft phase domain, a characteristic of a non-raft phase domain, and/or a characteristic of one or more membrane proteins based on the signal. Thus, user interface 205 can include a monitor or display screen to electronically display a signal or result. In some embodiments, user interface 205 can include a keyboard or other suitable device for accepting user input and/or a printer. In some embodiments, such as shown in FIG. 3E, system 200 can include a network or other communications interface (NIC) 207 (configured to provide communications access to entities external to system 200, e.g., other computing platforms, the internet, an internal network computer, etc.), one or more communication busses 208 for interconnecting the components of system 200, and a power supply 209 for powering the components of system 200.

In some embodiments, system 200 can include one or more additional non-volatile, non-transitory, computer readable memory devices or media (not shown) such as magnetic disk storage or persistent devices (e.g., memory means or storage means), which can optionally accessed by one or more controllers (not shown). Data in memory 206 can be seamlessly shared with the additional non-volatile memory using known computing techniques such as caching. Memory 206 or the additional non-volatile memory can include mass storage that is remotely located with respect to processor(s) 202. Thus, in some embodiments, some data stored in memory 206 or optional additional non-volatile memory can be hosted on computers that are external to system 200 but that can be electronically accessed by system 200 over an internet, intranet, or other form of network or electronic cable using network interface 207. For example, network interface 207 can be configured to send and/or receive information from internet/network 210, which can be in communication with one or more database and/or client computer, e.g., to retrieve information. In some embodiments, system 200 is a personal computer. The presently disclosed subject matter can also be performed using commercially available or custom hardware with dozens or more processors connected in parallel, at even greater speed.

In some embodiments, the additional non-volatile memory can store one or more databases that store programs, sample data sets, and the like, such as those referred to elsewhere herein with respect to the VesA software. In some embodiments, memory 206 stores an operating system that is configured to handle various basic system services and to perform hardware dependent tasks, and a network communications module that is configured to connect system 200 to various other computers such as remote curated data sources and/or to clients via one or more communication networks, such as the internet, other wide area networks, local area networks (e.g., a local wired or wireless network can connect the system 200 to the remote client), metropolitan area networks, and so on.

As noted above, memory 206 also can store a first module 206a and a second module 206b configured to cause processor(s) 202 to execute the various steps of a method as described herein. For example, first module 206a can be configured to cause processor(s) 202 to detect a signal from the population of vesicles using a microscope, camera or a sensor communicatively coupled to the computing platform; and second module 206b can be configured to identify the candidate compound as having an impact on a characteristic of a lipid raft phase domain, a characteristic of a non-raft phase domain, and/or a characteristic of one or more membrane proteins based on the signal. First module 206a also can be configured to cause processor(s) 202 to store results within a database in the additional non-volatile memory. In some embodiments, processor(s) 202 are configured to output the results to user interface 205 and/or network interface 207. In some embodiments, system 200 is configured to receive a signal from a microscope, camera or other sensor, configured in communication with user interface 205 or via a networked source through network interface 207. In some embodiments, processor(s) 202 are configured to detect a signal from the population of vesicles using a microscope, camera or a sensor communicatively coupled to the computing platform; and to identify the candidate compound as having an impact on a characteristic of a lipid raft phase domain, a characteristic of a non-raft phase domain, and/or a characteristic of one or more membrane proteins based on the signal.

In some embodiments, a non-transitory computer readable medium comprising computer executable instructions embodied in a computer readable medium that when executed by a processor of a computer control the computer to perform steps for identifying, in some embodiments automatedly identifying, the candidate compound as having an impact on a characteristic of a lipid raft phase domain, a characteristic of a non-raft phase domain, and/or a characteristic of one or more membrane proteins comprising: contacting a population of vesicles with a candidate compound, wherein one or more vesicles in the population of vesicles comprises a membrane comprising a lipid raft phase domain and a non-raft phase domain and optionally one or more membrane proteins, wherein in a portion of the population of vesicles there is only a single detectable, e.g. visible, phase, and in a portion of the population of vesicles both the raft and non-raft phase domains are visible; detecting a signal from the population of vesicles; and identifying the candidate compound as having an impact on a characteristic of a lipid raft phase domain, a characteristic of a non-raft phase domain, and/or a characteristic of one or more membrane proteins based on the signal. By way of elaboration and not limiting, the presently disclosed subject matter comprises a step of contacting a population of vesicles, wherein one or more vesicles in the population of vesicles optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are detectable and distinct from one another, referred to as phase separated. In some embodiments, the non-transitory computer readable medium comprising computer executable instructions embodied in a computer readable medium that when executed by a processor of a computer control the computer to perform any method steps as disclosed herein.

In some embodiments, software and analytical methods are provided to facilitate high throughput identification of GPMVs, to determine the extent of lipid phase separation, and to quantify membrane protein partitioning between raft and non-raft phase domains. While this software was developed to facilitate analysis of HTS data, it can be used more generally for the analysis of GPMVs. An example of source code for the software is as follows. The source code and indeed any steps/methods/processes/algorithms disclosed herein can be embodied by a module, which can be stored in memory and executed by a hardware processor.

### IV. EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art. Any and all steps/methods/processes/algorithms disclosed in the EXAMPLES can be embodied by a module, which can be stored in memory and executed by a hardware processor.

### Example 1

### Conceptual Basis for the Screen

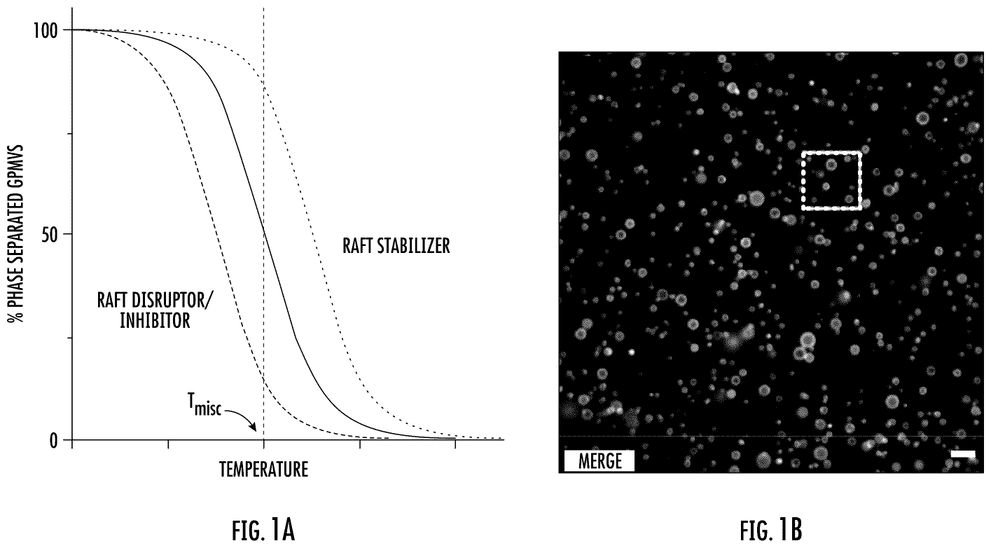

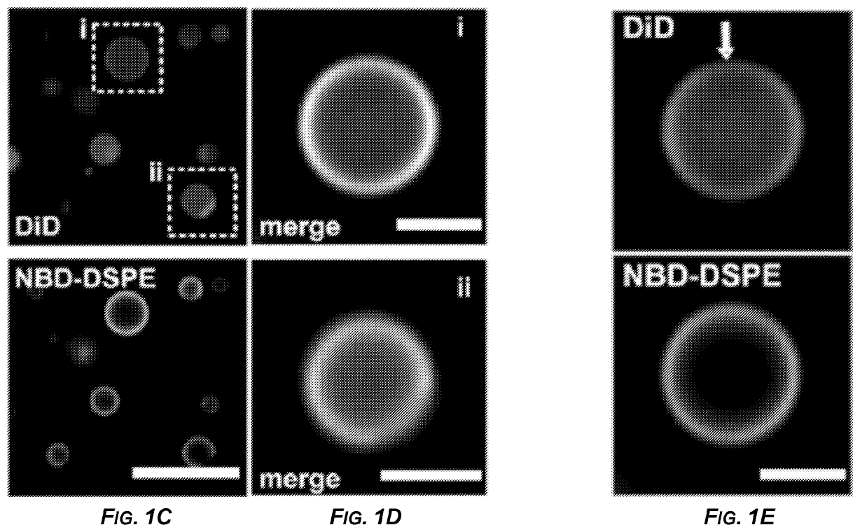

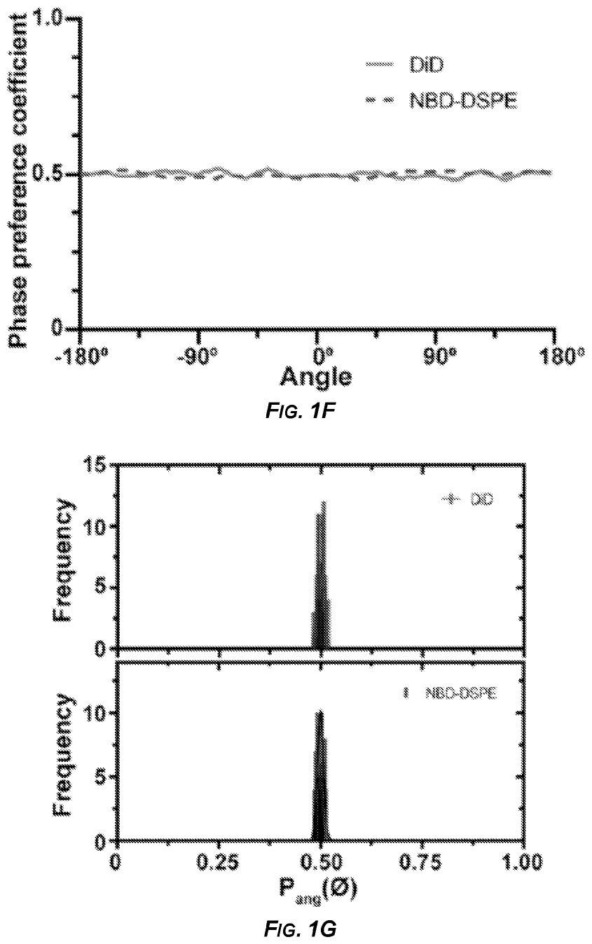

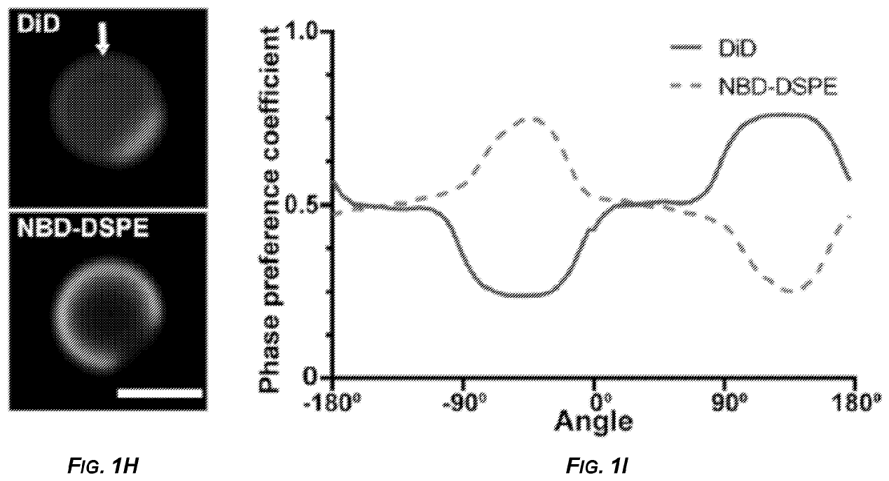

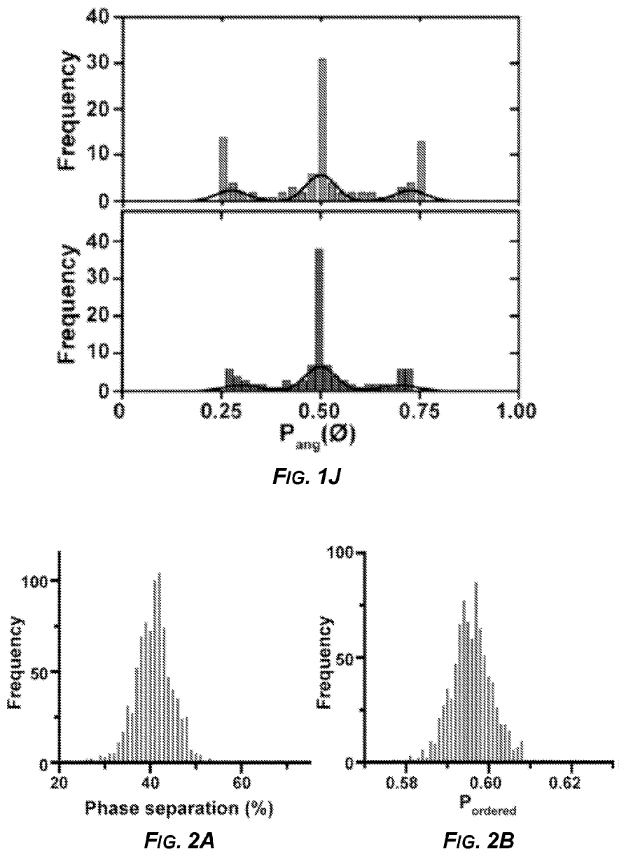

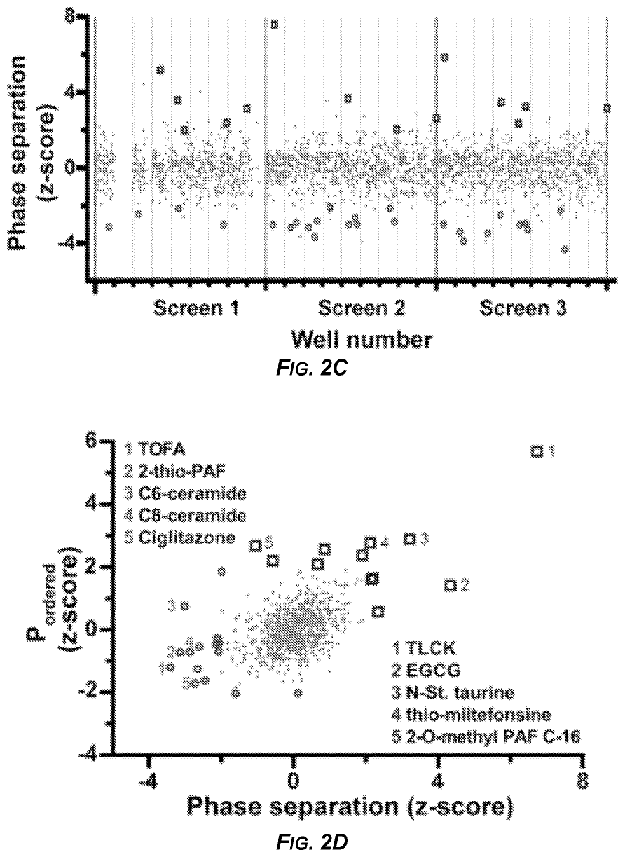

Classical measurements of T_{misc} are performed by measuring the percentage of phase separated GPMVs using fluorescence microscopy as a function of temperature (Gerstle et al., 2018). Effects of small molecules or other treatments on membrane phase behavior are then evaluated by comparing T_{misc} measurements obtained under each condition (Gray et al., 2013; Zhou et al., 2013; Raghunathan et al., 2015; Levental et al., 2016; Machta et al., 2016; Cornell et al., 2017; Gerstle et al., 2018). This approach is not practical for high throughput screening applications, which typically are performed at a single temperature. Changes in the percentage of phase-separated GPMVs were monitored at a single temperature in response to the addition of small molecules as a surrogate for measuring T_{misc} directly (FIG. 1A). This approach requires that a significant fraction of GPMVs are phase separated under control conditions, such that experimentally-induced changes can be readily detected. To fulfill this requirement we used GPMVs isolated from HeLa cells, which are known to yield a significant fraction of phase separated vesicles at room temperature (Johnson et al., 2010). Based on established criteria, compounds that increase the percentage of phase separated GPMVs are defined as raft stabilizers, whereas those that decrease the percentage of phase separated GPMVs function as raft disruptors/inhibitors of raft formation and lipid heterogeneity (Gray et al., 2013; Levental et al., 2016).

To visualize phase separation, GPMVs were labeled with the fluorescent lipid NBD-DSPE to mark the raft phase and DiD to mark the non-raft phase (Sezgin et al., 2012a). In phase separated vesicles these markers are enriched in their preferred domains, such that a given vesicle contains a co-existing NBD-DSPE-rich phase and a DiD-rich phase. In contrast, NBD-DSPE and DiD colocalize in a single uniform phase in GPMVs that are not phase separated. We also optimized conditions to isolate and plate GPMVs in multi-well plates. These modifications enabled us to capture images containing upwards of hundreds of GPMVs per field at high resolution (FIGS. 1B-1D), setting the stage for high content imaging.

### Example 2

### Development of an Automated Data Analysis Pipeline to Enable High Content Imaging of GPMVs

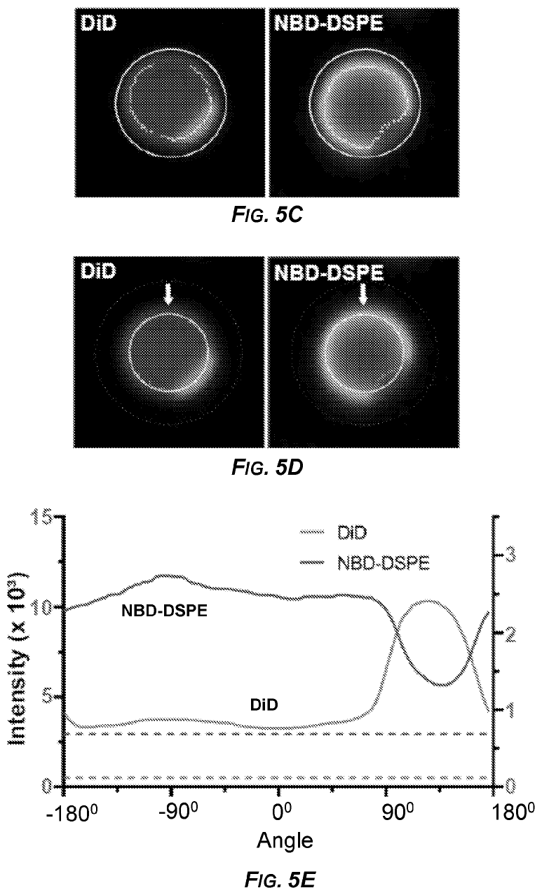

We next established a pipeline for GPMV data analysis. To date, analysis of GPMVs has been performed manually (Sezgin et al., 2012a; Lorent et al., 2017; Gerstle et al., 2018), but this approach is not feasible for high content imaging. A custom MATLAB-based pipeline was developed to automate GPMV image analysis based on an algorithm that automatically detects and analyzes individual GPMVs in fluorescence images (see FIG. 3A to 3E for an overview). It begins by identifying disc-shaped objects and excluding those that fail to meet certain selection criteria (FIGS. 4A to 4C). The position of the membrane contour for each accepted vesicle is then mapped and plots of fluorescence intensity along the vesicle membrane are generated (FIGS. 5A to 5E).

The algorithm next uses the fluorescence intensity information in the red and green channels to calculate a metric p_{ang}(ϕ) that quantifies the preference of the fluorescent markers for the ordered and disordered phases as a function of angle ϕ around the vesicle perimeter. We refer to this metric as a phase preference coefficient p_{ang}(ϕ). It is defined as

$${p}_{ang} ( ϕ ) = \frac{I ( ϕ )}{I ( ϕ ) + I ( ϕ + 18 {0}^{∘} )}$$

Here, I(ϕ) corresponds to the fluorescence intensity at angle ϕ and I (ϕ+180°) is the fluorescence intensity at angle ϕ+180°. This analysis assumes that the fluorescence intensity at a particular position is proportional to dye concentration. Each GPMVs is sampled at angles ranging from 0 to 180°, and 0 to −180°. In effect, this is the equivalent of drawing a line profile bisecting the GPMV at each angle ϕ, and using this information to calculate the percent of the dye present in the phase at angle ϕ. It is thus a generalized version of previously reported metrics reporting on the enrichment of fluorescent dyes or lipids in ordered domains such as % Lo, a measure of the preference of lipid probes for the Lo phase (Sezgin et al., 2012b) and % raft, a measure of the % of a given protein in the raft phase (Sezgin et al., 2012a). The main distinctions are that for the p_{ang}(ϕ) measurement i) the analysis is performed for every GPMV, ii) the bisecting lines are placed at every angle and are not limited to lines that cross through two different phases and iii) the analysis does not make an apriori assumption about which phase corresponds to the ordered or disordered domain.

For a GPMV in which a single phase is present, i.e. the distribution of red and green dye is uniformly distributed across all angles, p_{ang}(ϕ) yields a value of 0.5 (FIGS. 1E-1G). For a phase separated GPMV, typically three distinct p_{ang}(ϕ) values are obtained (FIGS. 1H-1J). For angles where the line bisects the same phase at angle ϕ and ϕ+180°, p_{ang}(ϕ) yields a value of 0.5. The other two values of p_{ang}(ϕ) report on the phase preference of the dye for the two distinct phases. When I(ϕ) corresponds to the brighter phase, p_{ang}(ϕ) will be >0.5. Conversely, when I(ϕ) corresponds to the dimmer phase, p_{ang}(ϕ) will be <0.5. Because each GPMV is sampled at angles ranging from 0 to 180°, and 0 to −180°, each position is sampled twice but from different directions (FIGS. 1H-1J).

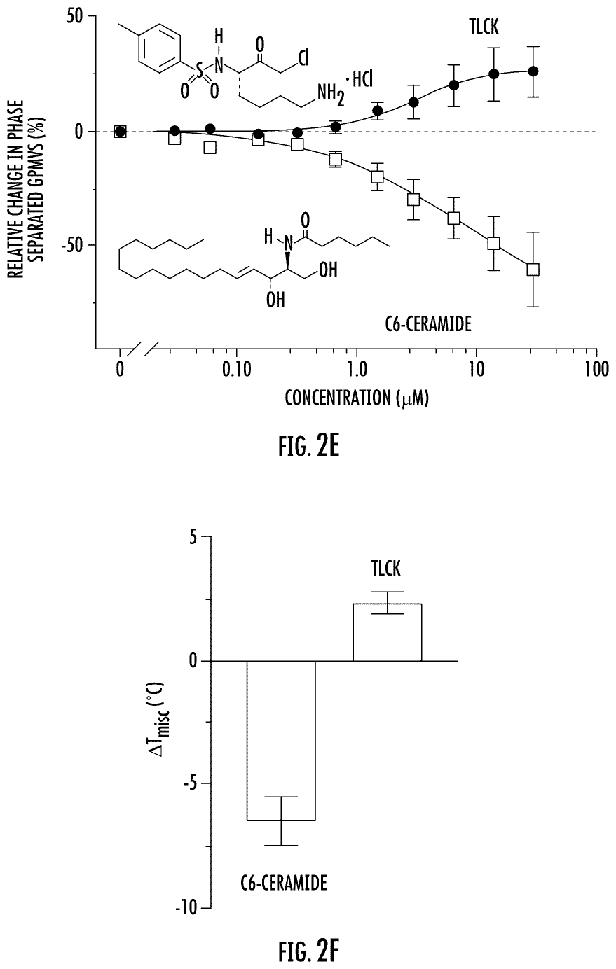

The program then generates histograms of p_{ang}(ϕ) for each GPMV and fits them to determine if they are best described by one Gaussian (single phase) or three Gaussians (phase separated), and the position of the maxima recorded. Each vesicle is then scored as phase separated or not depending on whether histograms of the partition coefficients comprise a single peak, indicating a single phase is present, or multiple peaks reflecting the presence of fluorophore-rich and fluorophore-poor domains (FIG. 1E-1J). Using this tool, it is possible to quickly identify and analyze hundreds of individual GPMVs per image across multiple images and wells of multi-well plates in an automated fashion. This enabled quantifying phase separation and phase preference coefficients for large vesicle populations, for example by averaging the percentage of phase separated vesicles and NBD-DSPE phase preference for the GPMVs detected in each well, and then generating histograms of these values across all the wells of a representative screen (FIGS. 2A and 2B).

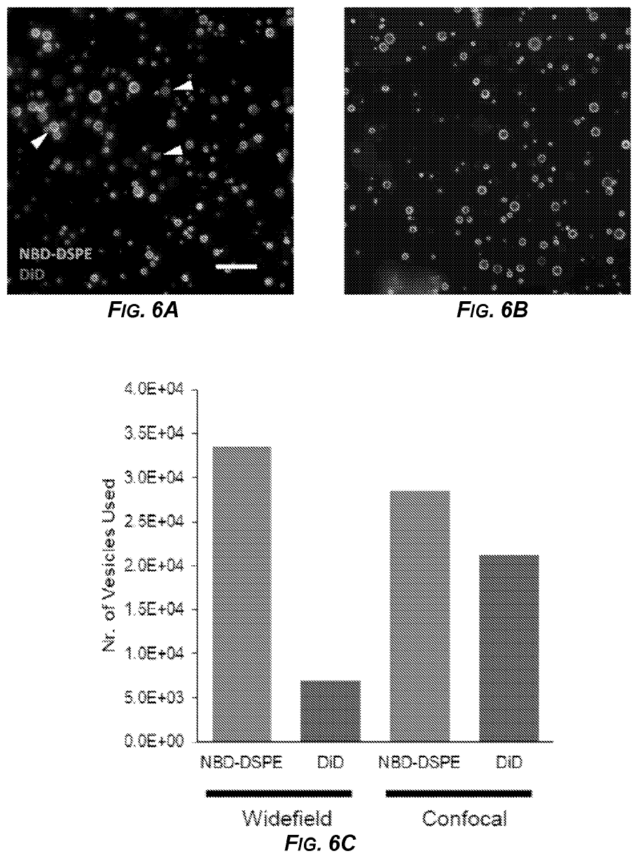

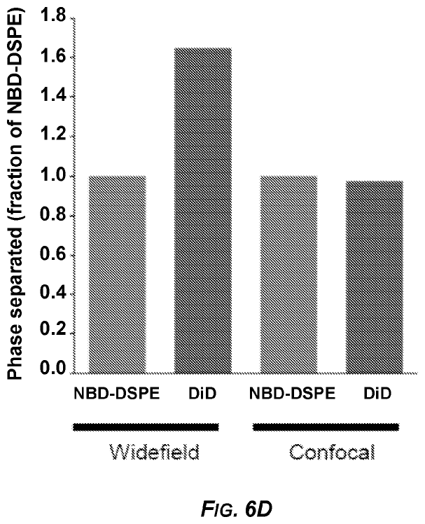

Because the data analysis pipeline employs detection of the percentage of phase separated vesicles, we next confirmed whether it could detect phase separated vesicles with equal efficiency in the red and green channels across multiple images (Table 3). For GPMVs that contain both red and green dyes, ˜90% were reported as being phase separated in the red channel, compared to ˜65% reported as phase separated in the green channel. This is due to the exclusion of a large percentage of red GPMVs from the final analysis, likely due to the relatively high level of DiD fluorescence in the lumen compared to the membrane contour (FIGS. 4A-4C). This appears to be a consequence of the wide field imaging approach used in the high content imaging microscope, as we found that if GPMVs were imaged using a confocal microscope that a similar percentage of vesicles were identified as phase separated in both the red and green channels (FIG. 6A to 6C). Given this systematic under detection of the vesicles in the DiD channel, only data from the green channel (NBD-DSPE) was used for subsequent data analyses of % phase separated vesicles and p_{ang}(ϕ). Because NBD-DSPE is a raft-preferring lipid probe, we report values of p_{ang}(ϕ)>0.5, which reflect its enrichment in the ordered phase. For simplicity, we will subsequently refer to this as P_{ordered}. P_{ordered} is essentially the equivalent of the previously reported % Lo metric (Sezgin et al., 2012b). In particular, % Lo=F_{Lo}/(F_{Lo}+F_{Ld}), where F_{Lo} and F_{Ld} are the fluorescence intensities of the reporter in the raft and non-raft phase domain, respectively (Sezgin et al., 2012b).

### Example 3

### A Proof-of-Principle Screen Identifies Established and New Chemical Modulators of Rafts

To provide proof-of-concept, the Cayman lipid library containing ˜850 bioactive lipid-like compounds in the Vanderbilt High-throughput Screening Facility was screened, using 1 mM of each compound across three independent screens. All screens were carried out using an automated microscope/high-content imaging system and robotic plate handler at RT. After applying vesicle selection criteria, the final analysis was performed on ˜2×10⁶ GPMVs, corresponding to an average of 868±437 vesicles per compound. Compounds were initially scored based on their capacity to alter the percentage of phase separated vesicles (FIG. 2A). Seven compounds were found to significantly increase the percentage of phase separated vesicles (z-score >2) while 14 decreased the percentage of phase separated GPMVs across at least two independent screening trials (Table 1, Table 4). Consistent with previous reports (Chiantia et al., 2007; Holowka et al., 2018), short chain ceramide analogs were consistently identified as raft disruptors that decreased the percentage of phase separated vesicles. Acylated taurines were among the candidates that increased the percentage of phase separated vesicles, i.e. stabilized rafts. A protease inhibitor, tosyl-L-lysyl-chloromethane hydrochloride (TLCK), was also identified as a potent raft stabilizer in the GPMV model.

Since the raft phase domain preference of lipid probes are known to be sensitive to the physical properties of membrane phases (Sezgin et al., 2012b), it was also examined whether these are affected by the compounds. Ten compounds significantly altered the phase preference of NBD-DSPE (z-score >2) (FIG. 2B, 2F; Table 2, Table 4). Of the 850 compounds screened, three compounds (TLCK hydrochloride, N-stearoyl taurine, and thio-miltefosine) significantly affected both the percentage of phase separated vesicles and the phase preference, while the other compounds affected only one parameter or the other (Tables 1-3). Together, these results indicate that some but not all bioactive lipids modulate membrane phase behavior, with varying modes of action.

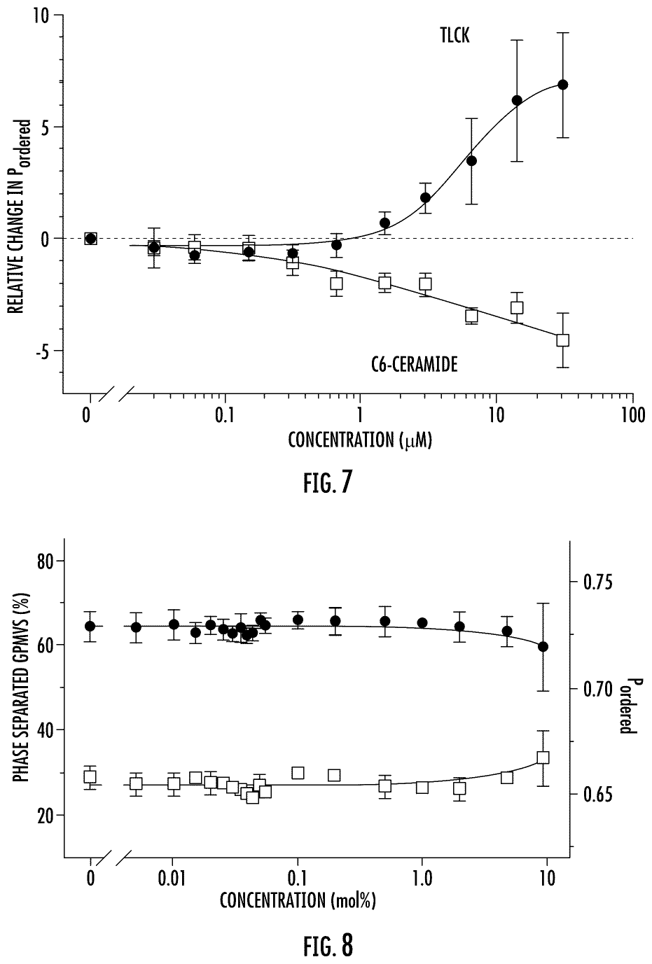

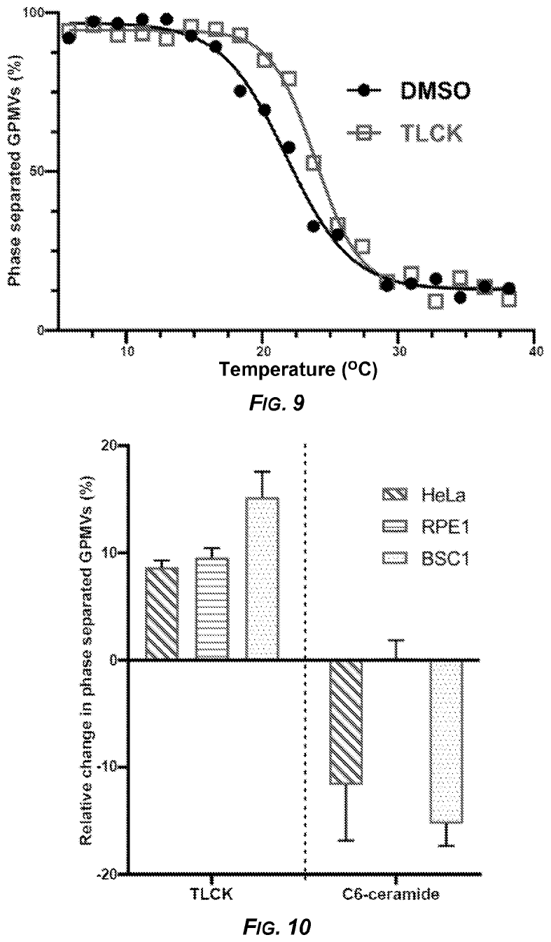

To validate the results of the screens, two hits were selected for deeper analysis: the short-chain ceramide analog C6-ceramide and the protease inhibitor TLCK (FIGS. 2E, 2F). In dose-response assays, using freshly made reordered stocks of each compound, TLCK systematically increased the percentage of phase separated vesicles as a function of increasing concentration, while the opposite effect was observed for increasing concentrations of C6-ceramide (FIG. 2E). Their effects on the phase preference of NBD-DSPE were also dose-dependent (FIG. 7). In contrast, the vehicle DMSO had little effect on the percentage of phase separated vesicles or phase preference except at the highest concentration studied, 10 mM (FIG. 8).

Next, the two candidates, TLCK and C6-ceramide, were independently validated by determining changes in T_{misc} relative to DMSO. For these experiments, the percentage of phase separated vesicles was measured as a function of temperature (Gerstle et al., 2018). As expected, TLCK increased T_{misc}, whereas C6-ceramide lowered T_{misc} (FIG. 2F, FIG. 9). We also assessed their effects in several different cell types (FIG. 10). Thus, these compounds represent bona fide examples of raft modulators in the GPMV model.

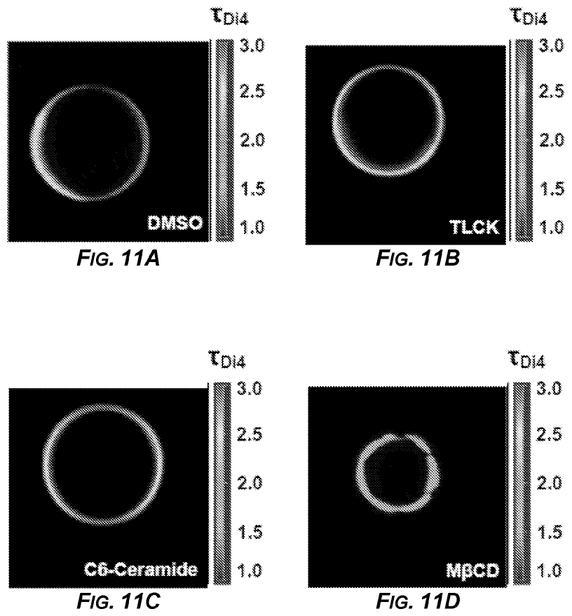

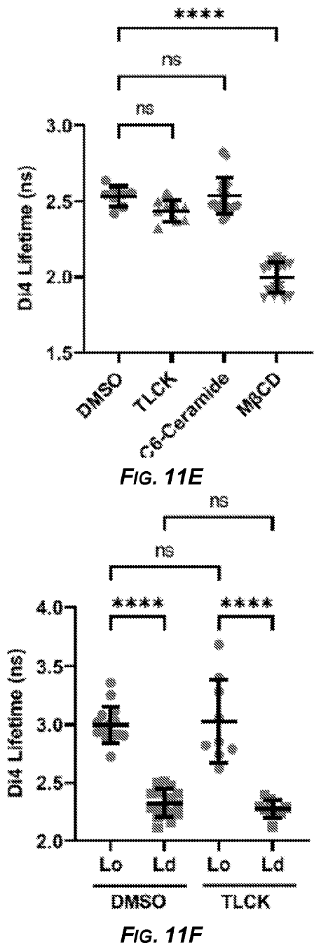

It was also of interest how TLCK and C6-ceramide impact other properties of the membrane compared to treatments such as cholesterol depletion that are commonly used to disrupt rafts in cells (Zidovetzki & Levitan, 2007). To test this, we examined their effects on membrane lipid packing and fluidity using a fluorescent reporter of membrane packing, Di-4-ANEPPDHQ (Di4) (Jin et al., 2006; Owen et al., 2006; Sezgin et al., 2014; Levental et al., 2020b; Lorent et al., 2020). As a control, we subjected GPMVs to cholesterol depletion using 5 mM MβCD. The photophysical characteristics of Di4, including its lifetime, are dependent on lipid packing in membranes. In particular, a lower Di4 lifetime is indicative of loose packing and a more fluid membrane environment whereas higher Di4 lifetimes point to a less fluid membranes and tighter lipid packing. Fluorescence lifetime microscopy (FLIM) images showed GPMVs treated with vehicle (DMSO) or TLCK comprised a mixture of phase separated (FIGS. 11A and 11B) and non-phase separated vesicles (not shown), whereas the majority of GPMVs treated with C6-ceramide contained a single membrane phase (FIG. 11C) when imaged at room temperature. The phase separated vesicles were characterized by two populations of lifetimes, corresponding to the Lo phase (higher lifetime) and Ld phase (lower lifetime) (FIGS. 11A-11F). In contrast, most MβCD-treated GPMVs contained multiple domains with the same lifetime (FIG. 11D), suggesting uniform lipid packing.

The average Di4 lifetimes for single-phase GPMVs treated with TLCK (2.43±0.06 ns) or C6-ceramide (2.53±0.11 ns) were similar to control GPMVs (2.53±0.06 ns), suggesting TLCK and C6-ceramide have little effect on overall membrane fluidity at the concentrations examined here (FIG. 11E). In contrast, average Di4 lifetimes were significantly lower in MβCD-treated GPMVs (1.99±0.09 ns), consistent with the expected decrease in membrane order arising from cholesterol depletion (FIG. 11E). We also examined the lipid packing of raft (Lo) and non-raft (Ld) domains across multiple DMSO and TLCK-treated GPMVs, and found that Di4 lifetimes were similar across treatments (FIG. 11F). Thus, despite their ability to significantly alter membrane phase behavior in the HTS assay, TLCK and C6 ceramide have substantially less perturbing effects on lipid packing and/or membrane fluidity compared to the effects of cholesterol depletion under the conditions of these experiments.

### Example 4

### SSMD* Metric Establishes the High Content Imaging Screen as a Robust Tool to Identify New Raft Modulators

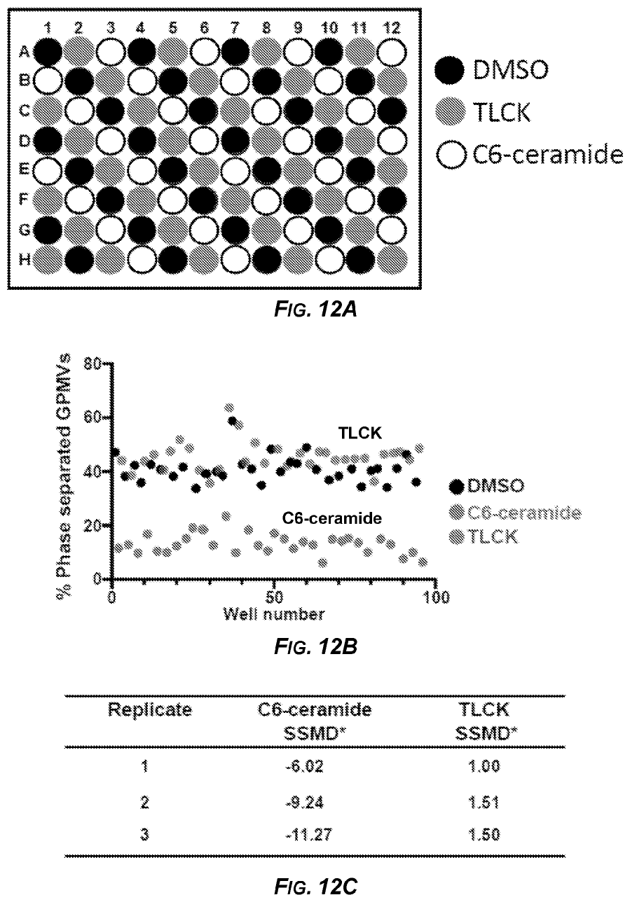

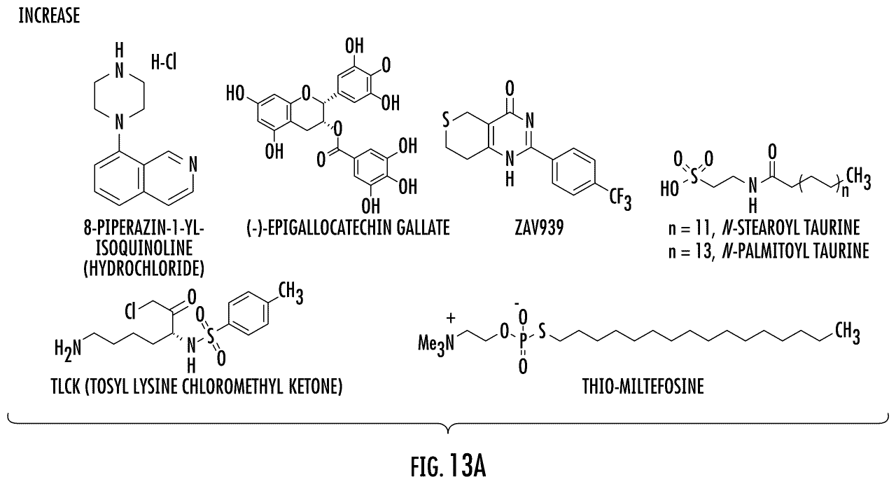

Finally, C6 ceramide and TLCK were used as positive controls to assess the quality and reproducibility of our assay. The use of the percentage of phase separated vesicles as our metric restricts the dynamic range and the use of standard HTS assay metrics, such as Z-prime (Zhang et al., 1999; Inglese et al., 2007). It was therefore chosen to calculate the robust strictly standardized mean difference (SSMD*) to evaluate assay performance and quality for HTS. This metric uses medians and median absolute deviations to estimate effect size and variability (Zhang, 2007; Zhang, 2011). Based on their effects on the percentage of phase separated GPMVs, we obtained a SSMD* of 8.84±2.65 for C6 ceramide and 1.34±0.29 for TLCK (mean±SD, n=3) (FIGS. 12A-12C), corresponding to extremely strong and moderate effects, respectively (Zhang, 2011). The SSMD* measurements thus confirm the robustness of the assay for HTS readiness and establish C6 ceramide and TLCK as useful positive controls for future screens.

### Supporting Information for EXAMPLES 1-4

Supporting information includes the following: methods; quantification of the efficiency of detection of phase separated GPMVs using DiD fluorescence versus NBD-DSPE fluorescence across screens; metrics for the hits; a representative figure illustrating the automated vesicle selection process; a description of contour detection and refinement of membrane position of individual GPMVs; an analysis of the percentage of phase separated vesicles detected in the DiD and NBD-DSPE channels using wide field versus confocal imaging; a dose response curve showing the effects of TLCK and C6-ceramide on P_{ordered} for NBD-DSPE; dose-response curves showing the effects of DMSO on the percentage of phase separated vesicles and P_{ordered} for NBD-DSPE; a representative temperature scan illustrating the effect of TLCK on T_{misc}; quantification of the effects of TLCK and C6-ceramide on GPMVs isolated from several different cell lines; analysis of the impact of TLCK, C6-ceramide and MβCD on lipid packing in GPMVs using Di4 lifetime measurements; results of experiments designed to test the HTS assay robustness based on SSMD* calculation; chemical structures of hits from the bioactivity lipid library that impact the percentage of phase separated GPMVs; chemical structures of hits from the bioactivity lipid library that impact P_{ordered} for NBD-DSPE; a description of how phase separated GPMVs are identified using the “colocalization” approach; an overview of approaches used to correct for positional effects and calculate Z-scores; and a user guide describing the functionality of the VesA software.

GPMV Preparation for High Throughput Screening

HeLa cells were obtained from ATCC (cat #CCL-2) and cultured at 37° C. in 5% CO₂ in a humidified tissue culture incubator. HeLa cells were maintained in DMEM (Gibco cat #11885-084) supplemented with 10% fetal bovine serum (FBS, Gibco 26140-079), 1% penicillin/streptomycin (P/S), and 1% L-glutamine. GPMVs were prepared from HeLa cells using the DTT/PFA method as described in Sezgin et al., 2017. In brief, cells were plated in 150 mm tissue culture dishes the day before the experiment, to yield a confluence of 70-80% the day of the experiment. To generate GPMVs, cells were rinsed twice in 10 ml of GPMV buffer (2 mM DTT, 25 mM formaldehyde, 150 mM NaCl, 10 mM HEPES, 2 mM CaCl₂), pH=7.4) and subsequently incubated in 12 ml of GPMV buffer at 37° C. with gentle shaking at 70 rpm for 1.5 h. The raft marker NBD-DSPE (1,2-distearoyl-sn-glycero-3-phosphoethanolamine-N-(7-nitro-2-1,3-benzoxadiazol-4-yl; Avanti Polar Lipids) and the non-raft marker DiD (1,1′-dioctadecyl-3,3,3′,3′-tetramethylindodicarbocyanine perchlorate; cat #D307 Thermo Scientific) were added from stock solutions in ethanol to the GPMV suspension at final concentrations of 5-6 μg/ml and 1 μg/ml, respectively, for the initial screenings. For some experiments, cells were prelabelled with 5 μg/ml DiD for 8 min at 4° C. and washed several times prior to GPMVs generation. Screening was performed the same day that GPMVs were generated.

High-Throughput Screening of GPMVs

High throughput screening was performed at the Vanderbilt High Throughput Screening Facility using their Cayman Bioactive Lipid Library (Cayman Chemical, cat #10506). Compounds dissolved in DMSO (stock concentration of 1 mM) were preloaded into 96-well plates using an acoustic droplet ejection liquid dispenser (ECHO 555, Labcyte) at volumes of 50-80 nl per well. 50-80 ml GPMVs suspension in GPMV buffer was then added to each well of a 96-well plate to obtain a final drug concentration of 1 μM. The GPMVs were allowed to settle to the bottom of the plates for 1.5 h prior to imaging. Samples were prepared and imaged at RT. The screen was repeated on three different days using three separately prepared batches of GPMVs (3 biological replicates).

Image acquisition was performed with an ImageXpress Micro XL automated microscope (Molecular Devices) using a Nikon 40X 0.6 NA Super Plan Fluor ELWD objective and a Lumencor Sola solid-state light source. Laser autofocus was employed at each site to obtain the best focus z-plane for each channel. NBD-DSPE fluorescence was excited and detected using a Semrock GFP filter set (GFP-3035B) consisting of a 472/30 excitation filter, 442-488/502-730 dichroic, and 520/35 emission filter, and DiD fluorescence was excited and detected using a Semrock Cy5 filter set (Cy5404A) comprising of a 628/40 excitation filter, a 594-691/669-726 dichroic, and 692/40 emission filter. Sixteen sites were captured per well using a 4.2 MP widefield scientific cMOS camera with a camera binning of 1. Each plate took approximately 1.3 h to image. Multiple plates were loaded to a plate carousel (ThermoScientific), shuttled to/from the ImageXpress via a ThermoScientific CRS F3 robot arm, and automatically run through the Momentum scheduling software (Thermo). To analyze the data, images and metadata were exported as TIFF files and subjected to image analysis using a custom MATLAB-based program as described below. Wells containing ≤50 useable GPMVs were excluded from further analyses.

Compounds identified in the initial screens as potential hits were re-ordered and their identity verified by mass spectrometry by the Vanderbilt Chemical Synthesis Core. Ten-point dose response curves using ˜1:2 serial dilutions were generated producing a range of final concentration of 30 nM to 30 mM and backfilled to yield equivalent concentrations of DMSO. Data were collected in duplicate for each concentration. Two independent dose response experiments were performed.

High-Content Image Analysis of GPMVs

To perform high content image analysis of GPMVs, we developed a custom algorithm called VesA using MATLAB (MathWorks). The program, installation instructions, and a quickstart user guide, are described herein. VesA automatically loads images, detects GPMVs, records their position and diameters, measures the fluorescence intensity of the membrane, local background, and lumen, determines whether they are phase separated or not, calculates partition coefficients, and outputs the relevant data in tabular format. A flowchart describing the overall steps in data processing is provided in FIGS. 3A-3E, and the individual steps used to process the images described in more detail below.

Disc identification, contour detection, and vesicle exclusion criteria. In the initial step of the analysis, a 2D Gaussian filter was used to smooth the images. Disc-shaped objects were identified using the built in MATLAB two-stage circular Hough transform (FIG. 4B) and the location of each disc, its diameter, and its circularity were recorded.

For each detected disc, the position of the membrane contour was refined by generating a radial intensity profile as a function of each angle 2πR, where R is the vesicle radius in pixels (FIG. 5A-5C). A circle was then fit into the position defined by the maxima of the radial intensity profile (FIG. 5D). This step provides a smoothing of the position of the membrane and also facilitates further analysis of phase separated vesicles, which by definition contain regions of both high and low membrane fluorescence. Bilinear interpolation was used to estimate the intensity on the circle at a given angle for each 2πR angle (FIG. 5E). The luminal (intravesicular) fluorescence, and the local background fluorescence were then measured and recorded for each circle together with its position and radius.

GPMVs were excluded from further analysis if they were closer than 5 pixels to one another, the fluorescence signal in the membrane was >98% of the maximum attainable intensity, the background fluorescence was >75% of the maximum membrane fluorescence, or if the interior (luminal) fluorescence vesicle was >85% of the maximum membrane fluorescence for a given GPMV. Additional constraints were also included to exclude vesicles where the magenta and green channels did not overlap due to drifting between imaging in the two channels, the center of the disks in the red and green channels were separated from each other by more than 10 pixels, or if there was more than a 15% deviation in the radii of the green and red channels for that same disk. This step also allows for correction of registration of GPMVs in the red and green channel that are offset due to drifting as well as re-scaling the disk radii to overlap to correct for small shifts in z that may occur between collection of the red and green images. Altogether, application of these criteria led to the exclusion of a significant fraction of GPMVs (for an example, compare FIGS. 4B and 4C).

Identification of phase separated vesicles and measurement of partition coefficients. Two methods were used to quantify the percentage of phase separated vesicles and partition coefficients.

In the first method, termed the “distribution” method in the software, the algorithm generates a series of line profiles bisecting the GPMV at each 2πR angle ϕ. The peaks in fluorescence intensity I corresponding to the position of the membrane along the line profile are then used to calculate angular partition coefficients, p_{ang} (FIGS. 1F, 1I) according to

$${p}_{ang} ( ϕ ) = \frac{I ( ϕ )}{I ( ϕ ) + I ( ϕ + 18 {0}^{∘} )}$$

Using this approach, non-phase separated vesicles yield a single population of angular partition coefficients centered around 0.5 (FIG. 1F), whereas phase separated vesicles typically yield three populations of partition coefficients (FIG. 1I). To computationally distinguish between these possibilities for each vesicle, histograms of angular partition coefficients were generated, and the histograms fit to determine if they were best described by one or three Gaussians. Each vesicle was then scored either as single phase (one Gaussian peak) (FIG. 1G) or phase separated (two or more symmetric Gaussians peaks) (FIG. 1J), and the partition coefficients were determined from the position of the maxima of the peaks. GPMVs in which a single Gaussian peak was obtained that was not symmetric around 0.5 were excluded from further analysis. This method would be expected to underestimate the fraction of phase separated vesicles since the measurements are made in a single focal plane bisecting the GPMVs. However, this effect should be consistent across wells and treatments.

A second approach termed the “colocalization” method was used to score the vesicles as phase separated or not for experiments where the average radius of the GPMVs was less than 10 pixels (FIGS. 15A-15F). In this method, the fluorescence intensity in the red and green channels were measured at each point along the membrane contour (FIGS. 15B and 15E). The intensity of red versus green fluorescence was then plotted for each datapoint (FIGS. 15C and 15F). For phase separated vesicles, the datapoints fall into two separate quadrants corresponding to pixels with high red signal but low green signal, and vice versa (FIG. 15C), whereas for membranes containing a single uniform phase, all the datapoints fall on the diagonal (i.e. they are colocalized) (FIG. 15F). This method was only used to score the percentage of phase separated vesicles and was not used to calculate partition coefficients.

Determination of % phase separated vesicles and partition coefficients per well, and correction for positional effects. For each plate, plots of the percent of phase separated vesicles per well were generated in the order they were imaged (FIG. 16A). Such plots typically yielded higher values in the initial wells that flattened to a plateau in the later part of the plate. This effect was observed across multiple plates, indicating it is not linked to the compounds within a specific plate but rather is related to changes that occur when plates are initially moved into the microscope. We speculate this may result from changes in temperature as each plate is moved into the sample chamber, which is at least 1° C. higher than RT, that level off once the plate temperature has equilibrated. To ensure our approach works irrespective of the cause of the position dependent effect, we implemented a correction that is believed to be helpful. To correct for these positional effects, the datasets for each well were fit with a simple exponential decay function

$${y}_{trend} = {y}_{0} \pm A {e}^{- ( \frac{x - {x}_{0}}{t} )}$$

where x is the well number and y is the percentage of phase separated vesicles for a single well. The data were then normalized to yield relative change my per well, Ay (FIG. 16B):

$$Δ y = \frac{y - {y}_{trend}}{{y}_{trend}}$$

Following these corrections, wells that exhibited either very high or very low relative changes in phase separation were apparent (FIG. 16C, 16D). A similar approach was used to correct for positional effects in measurements of partition coefficients.

Z-score determination and final ranking of hits. Z-scores of the percentage of phase separated vesicles (FIG. 16D) and partition coefficients were determined on a per-plate basis as

$$Z = \frac{Δ y}{{SD}_{plate} ( Δ y )}$$

Compounds with z-scores >2 or <−2 were deemed to be raft modulators. Some variability was observed between screens, in part because not all wells contained 50 usable GPMVs in every screen. Compounds that we identified as hits in at least 2 of the 3 independent screens were included in the final list of raft modulators.

Comparison of Widefield Versus Confocal Images

GPMVs were prepared from HeLa cells and labeled with NBD-DSPE and DiD as described above. GPMVs were added into a 96-well plate and imaged using an ImageXpress Micro Confocal High Content Screening System (Molecular Devices, San Jose CA) with a Nikon 40X 0.95 NA Plan Apo Lambda objective and an Andor Zyla 4.2 MP 83% QE sCMOS camera, and an 89-North LDI 5 channel laser light source. Nine fields were imaged per well. The whole plate was first imaged using a spinning disc confocal module with a 60 μm pinhole size. Immediately after the plate was imaged again in widefield. NDB-DSPE was excited and detected using a FITC filter set comprising Semrock a 460-488 nm excitation filter and 502-548 nm emission filter (widefield) or a 536/40 nm emission filter (confocal). DiD was excited and detected using a Cy5 filter set consisting of a 633-650 nm excitation filter and 653-730 nm emission filter (widefield) or a 692/40 nm emission filter (confocal). Imaging in each modality took approximately 45 minutes. Both image sets were analyzed in VesA as described above.

T_{misc} Measurements

GPMVs were generated from HeLa cells and either labelled with NBD-DSPE and DiI as described above (TLCK experiments) or labelled with DiI-C12 (C6-cermide experiments). They were then treated with 1 μM C₆ ceramide (Sigma Aldrich) or 10 μM TLCK (Cayman) for 1-2 h, mounted in a chamber formed from two coverslips separated by a silicon spacer in the continued presence of drug or an equivalent volume of DMSO (final concentration=0.1 mol %), and allowed to settle for 1 h prior to being imaged.

T_{misc} measurements for GPMVs treated with C6-ceramide were performed using a Zeiss LSM 510 confocal microscope. Images were collected using a 40X 1.2 NA Zeiss Plan-Neofluor objective with a confocal pinhole setting of 5 airy units. DiI-C12 fluorescence was excited using the 543 nm line of a HeNe laser and images were collected at 1× digital zoom. The final image resolution was 0.290 mm/pixel. A Linkam Peltier Cooling system (Tadworth, UK) was used to vary the temperature between 25 and 40° C. More than 5 fields of view were collected at each temperature. For these datasets, the number of phase separated GPMVs was scored manually. Data are reported as DT from 2 independent experiments.

T_{misc} measurements for GPMVs treated with TLCK were performed using a Zeiss A880 confocal microscope. At each temperature, a 3×3 tile of images was collected using 20× Air objective at a final pixel resolution of 0.138 mm/pixel. NBD-DSPE fluorescence was excited using a 458 Argon laser line and detected using a 458/548 dichroic and 482-614 nm band pass filter. DiD fluorescence was excited using a 633 nm HeNe laser and emission was collected using a 633/698.5 dichroic and a 638-759 nm band pass filter. A Peltier temperature system was used to vary the temperature between 5 and 38° C. For these datasets, the percentages of phase separated GPMVs were scored using the “colocalization” method using the MATLAB algorithm described above. Data are reported from 2 independent experiments.

Effects of Hits on GPMVs from Other Cell Types

GPMVs were generated from HeLa (Huma Cervical cancer cells, ATCC), hTERT-RPE-1 (Human Retinal Pigmented Epithelial cells, kindly provided by Dr. Ethan Lee, Vanderbilt University) and BSC1 (Monkey Kidney cells) (ATCC). Prior to GPMV preparation, cells were seeded in 150×25 mm plates and cultured in DMEM containing 10% FBS and 1% Penicillin/streptomycin in a tissue culture incubator with humidified air supplemented with 5% C02 at 37° C.

To prepare GPMVs, cells were washed twice with active GPMV buffer (2 mM DTT, 25 mM formaldehyde, 150 mM NaCl, 10 mM HEPES, 2 mM CaCl₂), pH=7.4) and plates with cells containing 10 ml of active GPMV buffer were incubated at 37° C. with gentle shaking at 90 rpm for 1.5 hours. GPMVs were collected and labeled with 9.7 μg/ml NBD-DSPE (Avanti Polar lipids #810141) and 1 μg/ml DiD (Thermo scientific #D307).

To test the effects of the putative hits and negative control compound, 1 μl of a 1 mM stock solution of TLCK or C6 ceramide, or an equivalent volume of vehicle (DMSO) was loaded per well in duplicate in a 96 well plate. 99 μl of GPMV suspension was added per well to obtain a final concentration of 10 mM of the compounds to be tested. After allowing the vesicles to settle down for 1-1.5 hours, they were imaged at RT using an Operetta CLS High Content Imaging System (Perkin Elmer HH16000000) fitted with 14-bit CMOS camera (1.3 Mega Pixel, 6.5 um/pixel). Epi-fluorescent images of 16 different sites of each well were acquired using a 40× water objective (NA 1.1). Fluorescence was excited with an LED light source and fluorescence emission was collected using Alexa 488 and Alexa 647 filter sets provided by the manufacturer. Data were exported as TIFF files and processed as described above. Data are reported from a single experiment performed in duplicate.

Di4 Fluorescence Lifetime Imaging

GPMVs were prepared as previously described by Sezgin, E, et al., Nat Protoc 2012, 7 (6), 1042-51. In brief, HeLa cells were washed twice with GPMV buffer (2 mM CaCl₂)/10 mM HEPES/0.15 M NaCl, pH 7.4), then washed twice with GPMV active buffer (GPMV buffer containing 25 mM formaldehyde and 2 mM DTT). Cells were then incubated with GPMV active buffer for 1-2 hours at 37° C. with shaking at 90-95 RPM. The GPMV-containing supernatant was decanted into a centrifuge tube and GPMVs were allowed to settle for 1 h. Aliquots of GPMVs (100 μL) were pipetted from the bottom of the Eppendorf tube into separate tubes and treated with DMSO (0.1%), 1 μM TLCK, 1 μM C6-ceramide or 5 mM MbCD. After 5 min, Di 4 ANEPPDHQ (Di4) (Thermo Scientific) was added to a final concentration of 1 mg/ml from a stock solution of 1 mg/mL Di4 in ethanol. The GPMVs were then sandwiched between two coverslips coated with 1 mg/mL bovine serum albumin (BSA) solution and allowed to incubate for an additional 20 min prior to lifetime measurements.

Di4 lifetime images were performed on a Leica TCS SP8 system comprising a Leica DMi8 inverted scanning confocal microscope with time-correlated single photon counting (TC-SPC) system and an 80 MHz-pulsed supercontinuum white light laser which allows continuously tunable excitation in the visible range coupled with filter-free detection. Samples were imaged using an HC PL APO CS2 63X/1.20 water objective. Di4 was excited at 488 nm and the emission was collected in the 550-800 nm range. The photon count rate was kept under 0.5 photons per laser pulse by adjusting the laser power, and sufficient frames were cumulatively acquired to obtain at least 100 photons per pixel. The fluorescence decay curves were fit with a bi-exponential re-convolution function adjusted to the instrument response function, and the average intensity-weighted lifetime was calculated for individual vesicles or area of interest and represented in the FLIM images as Di4 lifetime. Data are reported from two independent experiments. Statistical analyses were performed using GraphPad Prism 9.3.0.

Assessment of Assay Robustness Using SSMD*

GPMVs were prepared as described above and incubated for 2 hours at RT. They were then seeded into a flat bottom, black-walled 96-well plates (Greiner Bio-One, Kremsmunster, Austria) coated with 0.1% bovine serum albumin and pre-loaded in a checkerboard pattern with DMSO, 10 mM TLCK, or 10 mM C6 ceramide (FIG. 12A). GPMVs were imaged using the ImageXpress Micro XL automated imager using a Nikon 40× super plan fluor ELWD objective and analyzed using the software described above to determine the percentage of phase separated vesicles in each well (FIG. 12B). An estimate for robust strictly standardized mean difference (SSMD*) (Zhang, X. D., Genomics 2007, 89 (4), 552-611; Zhang, X. D., et al., J Biomol Screen 2011, 16 (7), 775-85) was then calculated based on the percentage of phase separated vesicles per well as reported in the NBD-DSPE channel according to the following equation:

$$S S M {D}^{*} = \frac{{{X}^{′}}_{P} - {{X}^{′}}_{N}}{\sqrt{{{s}^{′}}_{P} +} {{s}^{′}}_{N}}$$

Here, \acute{X}_{P} and \acute{X}_{N} represent the medians of the percentage of phase separated vesicles for the positive controls (C6 ceramide or TLCK) and negative control (DMSO), respectively, whereas ś_{P} and ś_{N} correspond to the absolute deviations from the median for the positive and negative controls. Three independent GPMV preparations were analyzed on separate days to evaluate reproducibility (FIG. 12C).

### Discussion of Examples

The foregoing Examples provide proof of concept for a high-throughput method to discover new chemical modulators of membrane rafts. In addition to short chain ceramides, several of the bioactive lipids identified here including the polyphenols epigallocatechin gallate and resveratrol as well as the alkylphospholipid analog miltefosine have previously been identified as raft modulators, demonstrating the validity and robust nature of our approach (Heczkova & Slotte, 2006; Adachi et al., 2007; Chiantia et al., 2007; Duran et al., 2012; Neves et al., 2016a; Neves et al., 2016b; Holowka et al., 2018; Tsuchiya & Mizogami, 2020; Zaremberg et al., 2020). Importantly, however, we also identified compounds that to our knowledge have not previously been shown to act on rafts in model membrane systems or cells, such as TLCK.

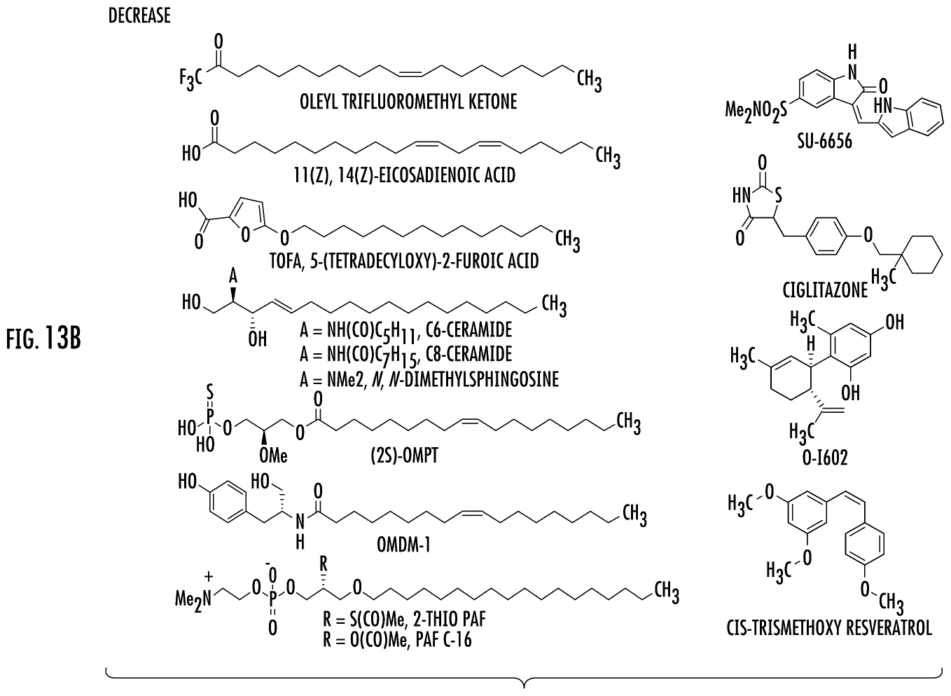

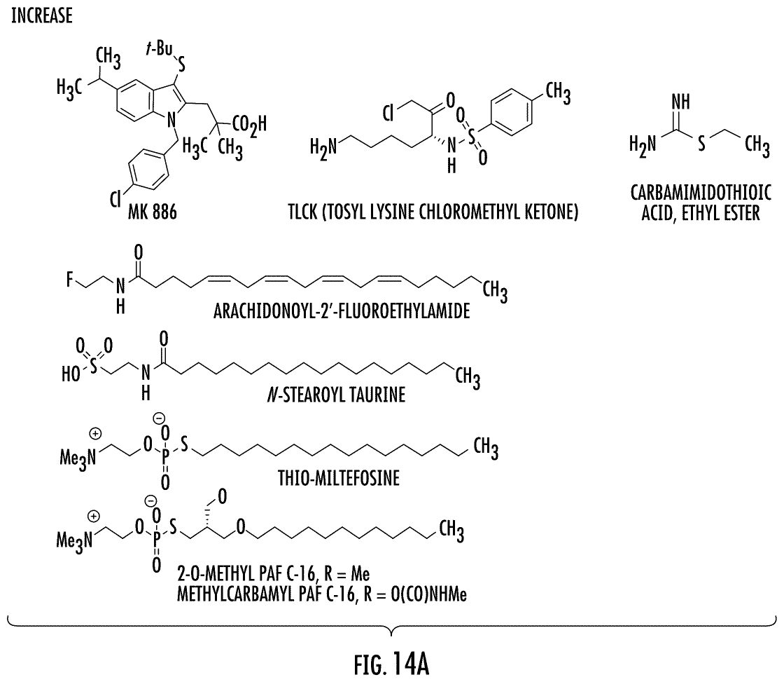

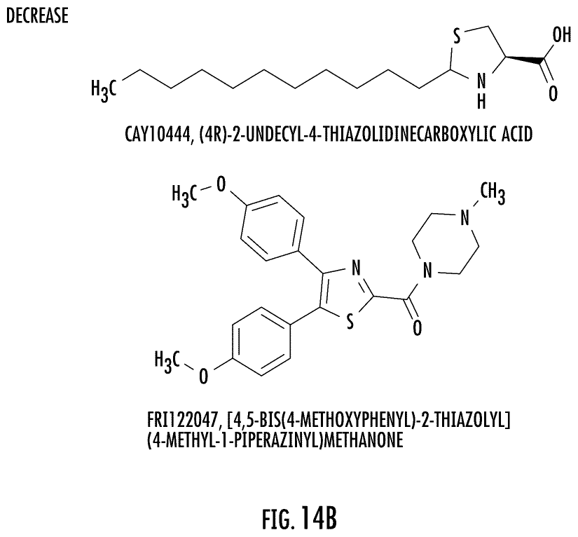

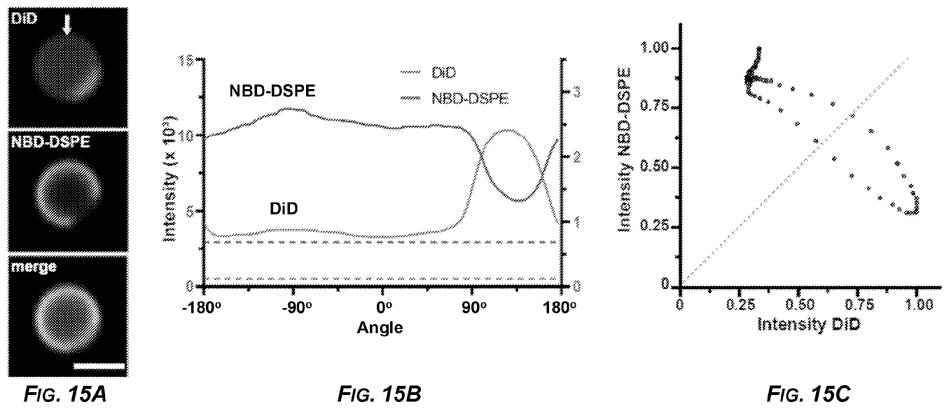

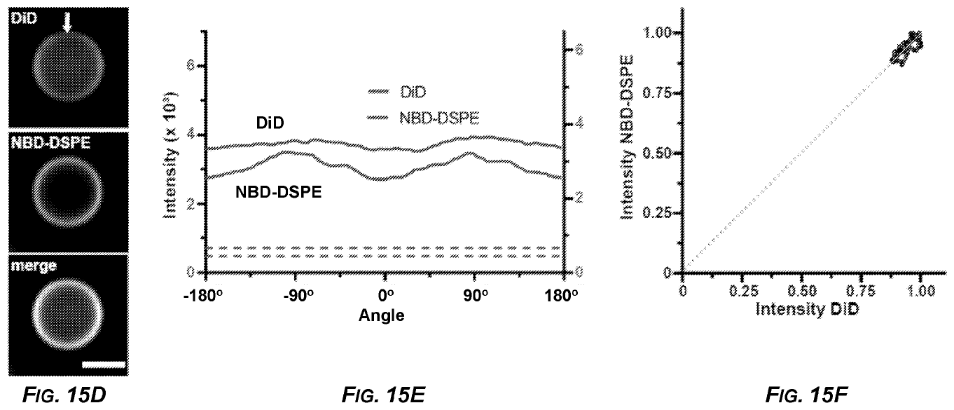

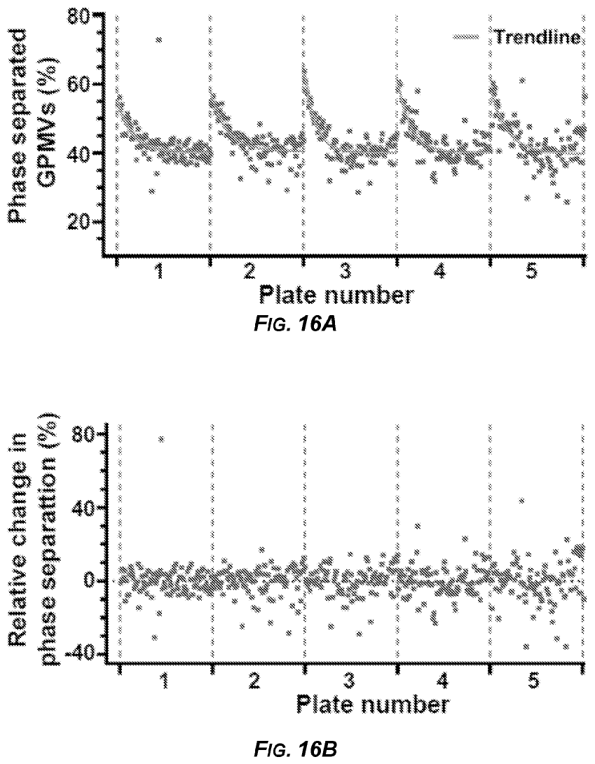

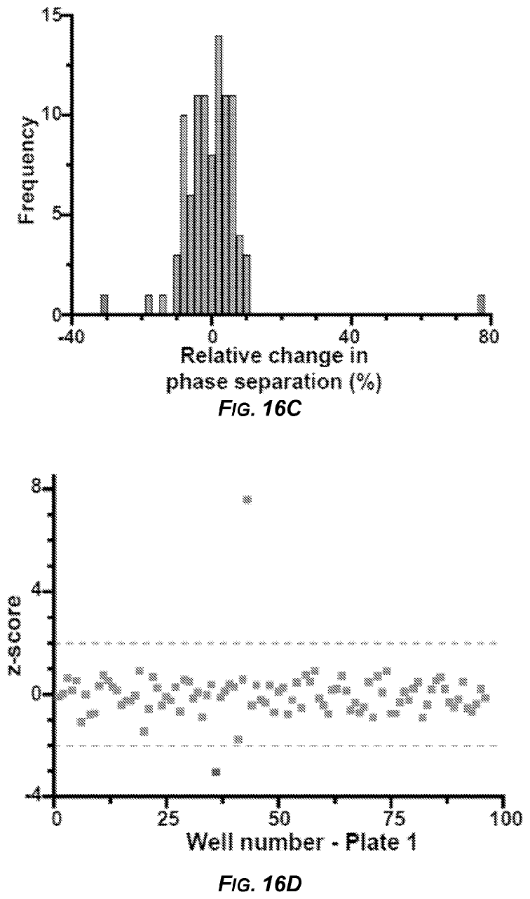

The hits varied in their impact on membranes. Some enhanced, while others inhibited membrane phase separation. The hits thus include examples of compounds that stabilize raft formation as well as some that inhibit raft formation. Some compounds affected both phase separation and lipid probe phase preference, suggesting they simultaneously affect lipid raft composition and behavior. Looking broadly across the hits, no overall trends relating charge or lipophilicity to activity were immediately evident (FIGS. 13A and 13B and 14A and 14B, Tables 1 and 2). However, the compounds that resulted in the strongest decreases in phase separation trended toward higher A Log P values compared to those that show a corresponding increase (FIGS. 13A and 13B, Table 1). Not surprisingly based on the composition of the bioactive lipid library, the bulk of the hit compounds have molecular architectures similar to fatty acids.

Together, these findings suggest that at least a subset of the hits impact domain formation by preferentially inserting into either the ordered or disordered phase, leading to changes in their physical properties and ultimately their miscibility. However, it is also possible that some of the hits influence membrane phase separation in other ways. For example, some of the non-fatty acid singleton hits, such as (−)-epigallocatechin, TLCK, resveratrol, and ciglitazone, contain chemical moieties that have been recognized to commonly yield false-positive screening results and/or are known to exhibit pleiotropic biological effects, including cell membrane perturbations (Baell & Holloway, 2010; Ingolfsson et al., 2014). Some compounds could potentially impact phase behavior indirectly by chemically modifying lipids or membrane proteins, similar to the behavior of some of the chemicals used to induce blebbing of GPMVs (Levental et al., 2011). It is possible for example that TLCK, which contains an electrophilic group, can covalently attach itself to some component of the membrane and thus induce the observed effects. Nevertheless, our initial studies suggest that the effects of C6-ceramide and TLCK on lipid packing are significantly less perturbing at the concentrations examined here than depleting cholesterol using MβCD, one of the most widely used approaches to experimentally disrupt rafts. Further testing which of these mechanisms are operative for each of these compounds, as well as evaluating their impact on raft-dependent biological activities, can also be done.

We also observed positional effects that are likely linked to temperature changes as the plates were loaded into the imaging chamber, an effect that could be corrected computationally. We also found that our data analysis algorithm underestimates the percentage of GPMVs labeled with DiD due to fluorescence signal in the interior of the GPMVs. We overcame this issue by relying on the NBD-DSPE fluorescence signal for data analysis, and showed that this effect can also be minimized experimentally using confocal imaging.

A number of immediate applications of this screening approach are provided, beyond extending it to other chemical libraries. As one example, it could be used to identify small molecules capable of modulating rafts without requiring changing levels of membrane cholesterol or sphingolipids, the current standard in the field. Since many clinically relevant membrane processes occur on the inner leaflet of the plasma membrane, this approach could be expanded to identify small molecules that act on inner leaflet phase separation using lipidated raft- and non-raft preferring proteins as markers (Stone et al., 2017). Examples of a disordered domain marker include a peptide anchored to the inner leaflet through a polybasic sequence and an attached geranylgeranyl moiety, whereas a minimal lipidated peptide with a saturated palmitoyl and myristoyl modifications preferentially associates with ordered domains (Pyenta et al., 2001; Baumgart et al., 2007; Stone et al., 2017). Given that rafts have been broadly implicated in human disease and are also exploited by pathogens (Verma, 2009; Hryniewicz-Jankowska et al., 2014; Sezgin et al., 2017; Bernardes & Fialho, 2018; Bukrinsky et al., 2020; Sviridov et al., 2020), candidates discovered through this approach could also be utilized to probe the pathophysiology of raft-associated diseases and possibly even be exploited for therapeutic applications. Our screening strategy could also be leveraged to identify small molecules that modulate the association of specific proteins with raft-like domains while leaving rafts themselves unperturbed. Finally, the high content image analysis pipeline described here should also have broad and immediate applications in studies utilizing GPMVs and other vesicle-based membrane models such as giant unilamellar vesicles. For example, it could greatly accelerate efforts to understand mechanisms that control the affinity of membrane proteins for raft domains (Lorent et al., 2017). Through these combined advances, it should become feasible to study, manipulate, and harness rafts in ways not previously possible.

TABLE 1
Compounds that significantly alter % phase separated GPMVs

| | | Effect on % | | | |
|---|---|---|---|---|---|
| Chemical | | phase separated | | | |
| Name¹ | CasRn | vesicles | z-score | ALogP | Function² |
| TOFA | 54857-86-2 | ↓ | −3.41 | 6.85 | A non-cytotoxic inhibitor of acetyl-CoA |
| | | | | | carboxylase and fatty acid synthesis |
| 2-thio-PAF | 96801-55-7 | ↓ | −3.15 | 4.02 | Isoteric analog of platelet activating factor |
| C6 Ceramide | 124753-97-5 | ↓ | −3.01 | 6.88 | Cell permeable analog of naturally |
| (d18:1/6:0) | | | | | occurring ceramide |
| C8 Ceramide | 74713-59-0 | ↓ | −2.87 | 7.79 | Cell permeable ceramide analog |
| (d18:1/8:0) | | | | | |
| Ciglitazone | 74772-77-3 | ↓ | −2.73 | 4.53 | Antidiabetic drug; potent and |
| | | | | | selective PPARγ ligand |
| Oleyl | 177987-23-4 | ↓ | −2.65 | 8.05 | Analog of oleic acid |
| Trifluoromethyl | | | | | |
| Ketone | | | | | |
| OMDM-1 | 616884-62-9 | ↓ | −2.62 | 7.59 | Endocannabinoid analog; inhibitor of |
| | | | | | arachidonoyl ethanolamide uptake |
| cis-trismethoxy | 94608-23-8 | ↓ | −2.45 | 3.77 | Antioxidant found in grapes and red wine |
| Resveratrol | | | | | with anti-proliferative, anti-neoplastic |
| | | | | | and anti-angiogenic activities |
| 11(Z),14(Z)- | 2091-39-6 | ↓ | −2.15 | 7.33 | Uncommon naturally occurring polyunsaturated |
| Eicosadienoic | | | | | fatty acid |
| Acid | | | | | |
| (2S)-OMPT | 1217471-69-6 | ↓ | −2.12 | 7.27 | Selective agonist of the lysophosphatidic |
| | | | | | acid 3 (LPA3) receptor |
| O-1602 | 317321-41-8 | ↓ | −2.10 | 4.79 | Abnormal cannabidiorcin; agonist of |
| | | | | | G-protein coupled receptor 55 |
| N,N- | 119567-63-4 | ↓ | −2.09 | 5.79 | Metabolite of sphingosine and an inhibitor |
| Dimethylsphingosine | | | | | of sphingosine kinase |
| (d18:1) | | | | | |
| SU6656 | 330161-87-0 | ↓ | −2.06 | 2.62 | Selective inhibitor of Src kinases |
| PAF C-16 | 74389-68-7 | ↓ | −2.00 | 3.46 | Platelet activating factor C-16; mediates |
| | | | | | neutrophil migration and reactive oxygen |
| | | | | | species production |
| TLCK hydrochloride | 4238-41-9 | ↑ | 6.73 | 2.18 | Non-selective proteinase inhibitor |
| (−)- | 989-51-5 | ↑ | 4.33 | 3.10 | Phenol found in green and black tea |
| Epigallocatechin | | | | | with diverse biological activities |
| Gallate | | | | | |
| N-Stearoyl | 63155-80-6 | ↑ | 3.21 | 6.29 | Amino-acyl endocannabinoid |
| Taurine | | | | | |
| XAV939 | 284028-89-3 | ↑ | 2.33 | 3.68 | Tankyrase inhibitor |
| N-Palmitoyl | 83982-06-3 | ↑ | 2.19 | 5.38 | Amino-acyl endocannabinoid |
| Taurine | | | | | |
| 8-piperazin-1-yl- | 936643-79-7 | ↑ | 2.14 | 1.17 | Synthetic intermediate used for |
| Isoquinoline | | | | | pharmacological synthesis |
| (hydrochloride) | | | | | |
| thio-Miltefosine | 943022-11-5 | ↑ | 2.12 | 4.47 | Analog of miltefosine, an inhibitor of |
| | | | | | phosphocholine cytidylyl transferase |
| | | | | | (CTP) with antimetastatic properties |

¹Abbreviations: TOFA: 5-(Tetradecyloxy)-2-furancarboxylic acid; 2-thio-PAF = 1-O-hexadecyl-2-deoxy-2-thio-S-acetyl-sn-glyceryl-3-phosphorylcholine; OMDM-1 = (S)-N-(1-(4-hydroxyphenyl)-2-hydroxyethyl) oleamide; (2S)-OMPT = 9Z-octadecenoic acid, (2S)-3-[(hydroxymercaptophosphinyl)oxy]-2-methoxypropyl ester, triethyl ammonium salt (1:2); O-1602 = 5-methyl-4-[(1R,6R)-3-methyl-6-(1-methylethenyl)-2-cyclohexen-1-yl] -1,3-benzenediol; SU6656 = 2,3-dihydro-N,N-dimethyl-2-oxo-3-[(4,5,6,7-tetrahydro-1H-indol-2-yl)methylene]-1H-indole-5-sulfonamide; PAF C-16 = platelet activating factor C-16 or 1-O-hexadecyl-2-O-acetyl-sn-glyceryl-3-phosphorylcholine; TLCK = Nα-Tosyl-Lys Chloromethyl Ketone; XAV939 = 3,5,7,8-tetrahydro-2-[4-(trifluoromethyl)phenyl]-4H-thiopyrano[4,3-d]pyrimidin-4-one
²As reported in Cayman product information

TABLE 2
Compounds that significantly alter NBD-DSPE raft phase preference

| | | Effect | | | |
|---|---|---|---|---|---|
| Chemical | | on | | | |
| Name¹ | CasRn | P_{ang}(ϕ) | z-score | ALogP | Function² |
| CAY10444 | 298186-80-8 | ↓ | −2.04 | 2.05 | Selective antagonist of the S1P₃/EDG3 receptor, |
| | | | | | a member of a family of G-protein coupled |
| | | | | | receptors that bind sphingosine-1 |
| | | | | | phosphate (S1P) |
| FR122047 | 130717-51-0 | ↓ | −2.03 | 3.79 | Selective inhibitor of COX-1 |
| (hydrochloride) | | | | | |
| TLCK | 4238-41-9 | ↑ | 5.69 | 2.18 | Non-selective proteinase inhibitor |
| hydrochloride | | | | | |
| N-Stearoyl Taurine | 63155-80-6 | ↑ | 2.89 | 6.29 | Amino-acyl endocannabinoid |
| thio-Miltefosine | 943022-11-5 | ↑ | 2.78 | 4.47 | Analog of miltefosine, an inhibitor of |
| | | | | | phosphocholine cytidylyl transferase |
| | | | | | (CTP) with antimetastatic properties |
| 2-O-methyl PAF C-16 | 78858-44-3 | ↑ | 2.68 | 3.49 | Synthetic platelet activating factor analog |
| S-ethyl Isothiourea | 1071-37-0 | ↑ | 2.57 | 0.77 | Potent inhibitor of nitric oxide synthase |
| (hydrobromide) | | | | | |
| Arachidonoyl-2′- | 166100-37-4 | ↑ | 2.38 | 6.88 | Analog of anandamide that binds |
| Fluoroethylamide | | | | | cannabinoid receptors |
| Methylcarbamyl | 91575-58-5 | ↑ | 2.20 | 3.46 | Stable analog of platelet activating |
| PAF C-16 | | | | | factor C-16 |
| MK-886 | 118414-82-7 | ↑ | 2.10 | 8.11 | Amino-acyl endocannabinoid |

¹Abbreviations: 2CAY10444 = 2-undecyl-thiazolidine-4-carboxylic acid; FR122047 = 1-[[4,5-bis(4-methoxyphenyl)-2-thiazolyl]carbonyl]-4-methyl-piperazine, monohydrochloride; MK-886 = 1-[(4-chlorophenyl)methyl]-3-[(1,1-dimethylethyl)thio]-α,α-dimethyl-5-(1-methylethyl)-1H-indole-2-propanoic acid
²As reported in Cayman product information

TABLE 3
Efficiency of detection of phase separated GPMVs using DiD
(red when presented in color) fluorescence versus NBD-DSPE
(green when presented in color) fluorescence across screens.

| | Mean number of | Phase-separated | Phase-separated |
|---|---|---|---|
| | GPMVs/well¹ | red GPMVs² | green GPMVs |
| Screen 1 | 232 ± 193 (697) | 89.4 ± 6.2% | 52.0 ± 8.6% |
| Screen 2 | 214 ± 98 (849) | 88.6 ± 3.9% | 69.1 ± 5.2% |
| Screen 3 | 172 ± 95 (806) | 94.5 ± 3.6% | 73.0 ± 6.7% |
| Average | 206 ± 139 (2355) | 90.8 ± 5.3% | 65.4 ± 11.2% |

¹Mean ± SD (n = number of wells) of the number of GPMVs that are both red and green.
²The large percentage of phase separated GPMVs in the red channel reflects systematic exclusion of red GPMVs after applying selection criteria using the image processing algorithm (see FIGS. 4A-4C for examples of GPMVs that are detected in the green channel but not the red channel).

TABLE 4
Metrics for hits

| | | | | | | | |
|---|---|---|---|---|---|---|---|
| | | | | No. of | % phase | | |
| | | | Found | used | separated | P_{ordered} | |
| Chemical Name¹ | CasRn | VU Number | disks | GPMVs | z-score | z-score | Repeats |

Compounds that decrease % phase separated GPMVs

| | | | | | | | |
|---|---|---|---|---|---|---|---|
| TOFA | 54857-86-2 | VU0454815 | 25663 | 3081 | −3.41 | −1.20 | 3 |
| 2-thio-PAF | 96801-55-7 | VU0454596 | 15924 | 1266 | −3.15 | −0.72 | 3 |
| C6 Ceramide (d18:1/6:0) | 124753-97-5 | VU0454551 | 16444 | 986 | −3.01 | 0.76 | 3 |
| C8 Ceramide (d18:1/8:0) | 74713-59-0 | VU0454580 | 11224 | 1551 | −2.87 | −0.72 | 2 |
| Ciglitazone | 74772-77-3 | VU0454703 | 24290 | 3578 | −2.73 | −1.71 | 3 |
| Oleyl Trifluoromethyl Ketone | 177987-23-4 | VU0454635 | 28111 | 2418 | −2.65 | −1.25 | 3 |
| OMDM-1 | 616884-62-9 | VU0454354 | 15245 | 838 | −2.62 | −0.54 | 3 |
| cis-trismethoxy Resveratrol | 94608-23-8 | VU0454575 | 31300 | 1657 | −2.45 | −1.61 | 2 |
| 11(Z),14(Z)-Eicosadienoic Acid | 2091-39-6 | VU0454750 | 28101 | 2363 | −2.15 | −0.46 | 3 |
| (2S)-OMPT | 1217471-69-6 | VU0454882 | 28604 | 2510 | −2.12 | −0.27 | 3 |
| O-1602 | 317321-41-8 | VU0454839 | 24490 | 2925 | −2.10 | −0.27 | 3 |
| N,N-Dimethylsphingosine (d18:1) | 119567-63-4 | VU0454609 | 25783 | 2020 | −2.09 | −0.70 | 3 |
| SU6656 | 330161-87-0 | VU0455031 | 21976 | 1628 | −2.06 | −0.42 | 3 |
| PAF C-16 | 74389-68-7 | VU0454565 | 17334 | 997 | −2.00 | 1.87 | 2 |

Compounds that increase % phase separated GPMVs

| | | | | | | | |
|---|---|---|---|---|---|---|---|
| TLCK hydrochloride | 4238-41-9 | VU0454477 | 9682 | 1781 | 6.73 | 5.69 | 2 |
| (—)-Epigallocatechin Gallate | 989-51-5 | VU0244129 | 14772 | 2379 | 4.33 | 1.42 | 2 |
| N-Stearoyl Taurine | 63155-80-6 | VU0454824 | 26313 | 2125 | 3.21 | 2.89 | 3 |
| XAV939 | 284028-89-3 | VU0454979 | 24084 | 1487 | 2.33 | 0.58 | 3 |
| N-Palmitoyl Taurine | 83982-06-3 | VU0454833 | 25582 | 3185 | 2.19 | 1.66 | 3 |
| 8-piperazin-1-yl-Isoquinoline | 936643-79-7 | VU0454937 | 22084 | 1464 | 2.14 | 1.61 | 3 |
| (hydrochloride) | | | | | | | |
| thio-Miltefosine | 943022-11-5 | VU0455039 | 29277 | 2406 | 2.12 | 2.78 | 3 |

Compounds that decrease NBD-DSPE partition coefficient

| | | | | | | | |
|---|---|---|---|---|---|---|---|
| CAY10444 | 298186-80-8 | VU0454788 | 24223 | 2407 | −1.61 | −2.04 | 3 |
| FR122047 (hydrochloride) | 130717-51-0 | VU0454334 | 14553 | 707 | 0.12 | −2.03 | 3 |

Compounds that increase NBD-DSPE partition coefficient

| | | | | | | | |
|---|---|---|---|---|---|---|---|
| TLCK hydrochloride | 4238-41-9 | VU0454477 | 9682 | 1781 | 6.73 | 5.69 | 2 |
| N-Stearoyl Taurine | 63155-80-6 | VU0454824 | 26313 | 2125 | 3.21 | 2.89 | 3 |
| thio-Miltefosine | 943022-11-5 | VU0455039 | 29277 | 2406 | 2.12 | 2.78 | 3 |
| 2-O-methyl PAF C-16 | 78858-44-3 | VU0454585 | 28677 | 1217 | −1.06 | 2.68 | 3 |
| S-ethyl Isothiourea (hydrobromide) | 1071-37-0 | VU0243742 | 12660 | 799 | 0.85 | 2.57 | 2 |
| Arachidonoyl-2′-Fluoroethylamide | 166100-37-4 | VU0454718 | 28628 | 2478 | 1.90 | 2.38 | 3 |
| Methylcarbamyl PAF C-16 | 91575-58-5 | VU0454566 | 18050 | 1523 | −0.58 | 2.20 | 3 |
| MK-886 | 118414-82-7 | VU0254002 | 14472 | 1337 | 0.67 | 2.10 | 2 |

¹Abbreviations: TOFA: 5-(Tetradecyloxy)-2-furancarboxylic acid; 2-thio-PAF = 1-O-hexadecyl-2-deoxy-2-thio-S-acetyl-sn-glyceryl-3-phosphorylcholine; OMDM-1 = (S)-N-(1-(4-hydroxyphenyl)-2-hydroxyethyl) oleamide; (2S)-OMPT = 9Z-octadecenoic acid, (2S)-3-[(hydroxymercaptophosphinyl)oxy]-2-methoxypropyl ester, triethyl ammonium salt (1:2); O-1602 = 5-methyl-4-[ (1R,6R)-3-methyl-6-(1-methylethenyl)-2-cyclohexen-1-yl]-1,3-benzenediol; SU6656 = 2,3-dihydro-N,N-dimethyl-2-oxo-3-[(4,5,6,7-tetrahydro-1H-indol-2-yl)methylene]-1H-indole-5-sulfonamide; PAF C-16 = platelet activating factor C-16 or 1-O-hexadecyl-2-O-acetyl-sn-glyceryl-3-phosphorylcholine; TLCK = Nα-Tosyl-Lys Chloromethyl Ketone; XAV939 = 3,5,7,8-tetrahydro-2-[ 4-(trifluoromethyl)phenyl]-4H-thiopyrano[4,3-d]pyrimidin-4-one; CAY10444 = 2-undecyl-thiazolidine-4-carboxylic acid; FR122047 = 1-[4,5-bis(4-methoxyphenyl)-2-thiazolyl]carbonyl]-4-methyl-piperazine, monohydrochloride; MK-886 = 1-[(4-chlorophenyl)methyl]-3-[(1,1-dimethylethyl)thio]-α,α-dimethyl-5-(1-methylethyl)-1H-indole-2-propanoic acid

### Example 5

VesA installation guide and representative requirements Operating system and software requirements
VesA has been tested on multiple operating systems including Windows 10 Enterprise, Linux, CentOS 7, and macOS Catalina.
Required software: MATLAB
   Required MATLAB plugins:
      ‘Image Processing Toolbox’
      ‘Curve Fitting Toolbox’
      ‘Parallel Computing Toolbox’
Typical installation time (assuming MATLAB has already been installed) is less than 1 min.

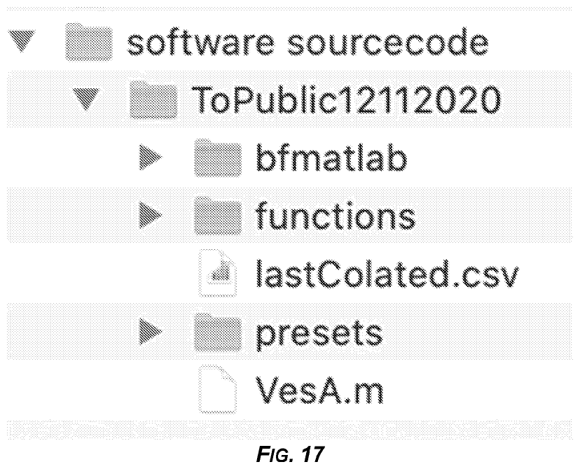

Installation Instructions:
1. Copy the zipped “VesA” folder and “DemoSingleImage” (demo dataset) folder onto desktop.
2. Unzip both folders.
3. Locate the VesA.m file in the “Software sourcecode” folder. See FIG. 17.
4. Double click on VesA.m. This will automatically launch MATLAB and load the VesA code.
5. To start the software, click on the “Run” button in MATLAB (highlighted below).

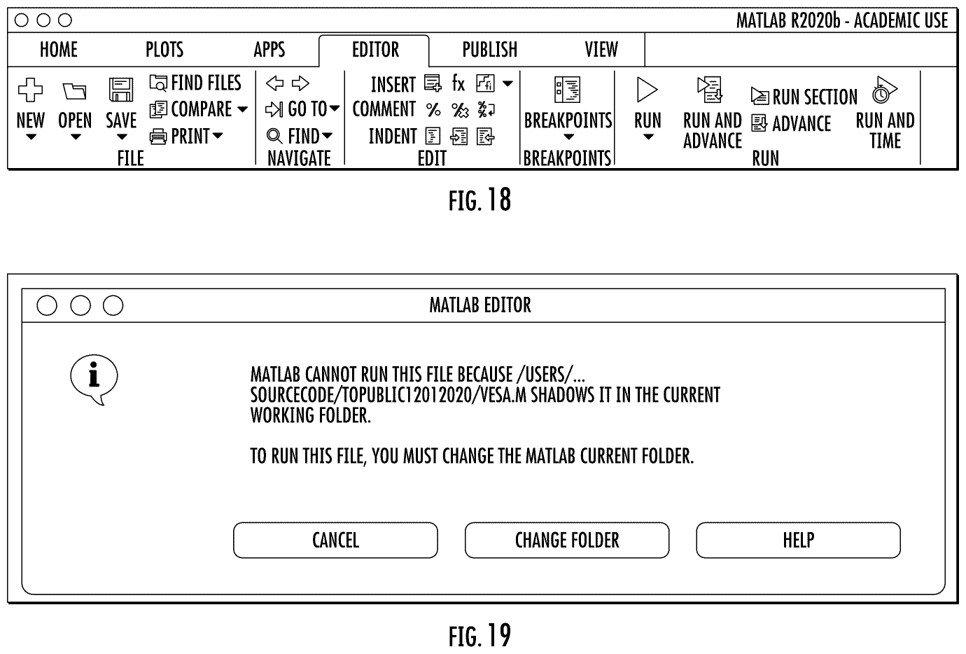

This will launch the VesA GUI. See FIG. 18.

Troubleshooting:

A user may receive the following message when first launching VesA in MATLAB. If received, select “Change Folder”. See FIG. 19.

Preface
VesA is designed to analyze single vesicles, populations of vesicles within a single image, stacks consisting of multiple images (“well”), or sets of image stacks (“plate”).
A wide range of image types can be analyzed, including microscope-specific formats.
Information from image headers is automatically loaded.
Images can include up to three channels; for demonstration purposes, two-channel images are used.
VesA automatically displays channel 1 as red, channel 2 as green, and channel 3 as blue.
The steps required to analyze images are organized under series of tabs. Starting from left to right, they include:

### “Database”=>“Detect Vesicles”=>“Analyze Vesicles”=>“Results Vesicles”=>“Plates Summary”

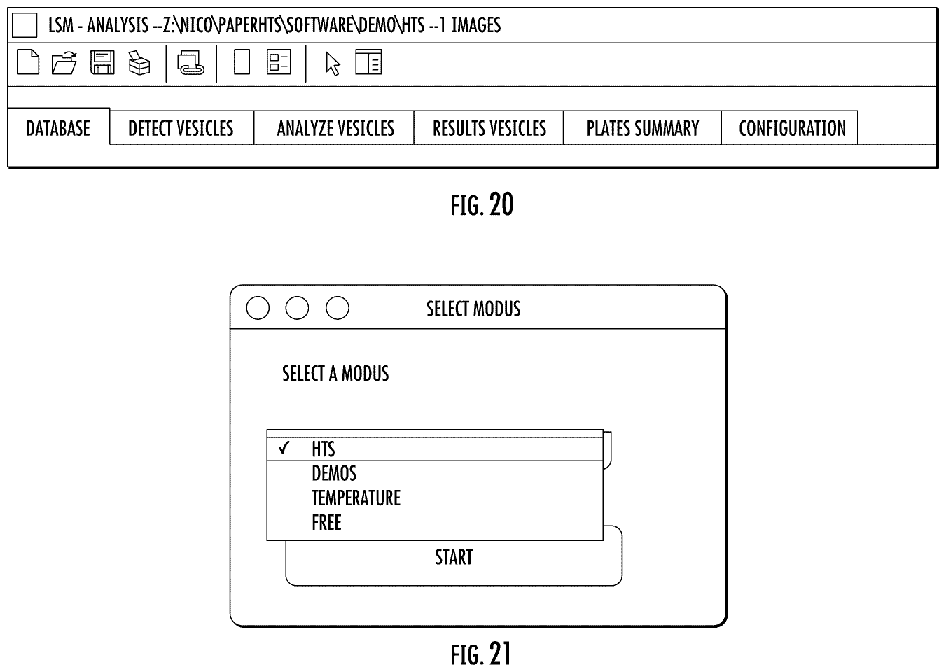

The final tab, “Configuration”, allows for saving or loading previously saved parameters for data analysis. See FIG. 20.
Three demo datasets are available. They comprise real experimental data containing GPMVs labeled with red and green dyes, collected under conditions identical to that analyzed as described herein:
   A single image (“DemoSingleImage” folder, provided with software.)
   A stack of images (“DemoStackImages” folder, available upon request.)
   A demo dataset consisting of a series of image stacks collected from different wells in a multiwell plate from a dose-response experiment (“demo/DemoSetOfStacks”, available upon request).
Expected results for each demo dataset are included in the “Expected results” folder and consist of sets of tables and a screenshot from the “Results vesicles” tab.
Instructions on how to analyze the demo datasets are provided in italics at the bottom of the description of the functionality of each tab in the VesA software.
The software uses three methods to calculate “partition coefficients.” Two of these methods (distribution method and colocalization method) are described in more depth in accompanying paper. A third method, termed the threshold method, is also available for use in the software at the user's discretion.
For simplicity, the term “partition coefficient” is used in this manual and software to describe the preference of markers for the ordered versus disordered phases. For the case of the distribution analysis, this corresponds to the “phase preference coefficient” or “P_{ordered}” described elsewhere herein.

1. Launching VesA
Please see accompanying “Read Me” file for VesA installation guide and requirements
When starting VesA, a window will pop up enabling several different preset parameters to be chosen.
The “Demos” setting will load preset parameters to analyze the demo images.
“HTS” will load the settings used to analyze HTS data collected from 96-well plates using the Operetta microscope as described elsewhere herein.
“Temperature” loads settings used to collect data from the temperature scans collected using a Zeiss 880 reported elsewhere herein.
The “Free” setting enables the user to freely define parameters used for data analysis.
See FIG. 21.
Demo:
For demo1, demo2, or demo3 choose “demo”.

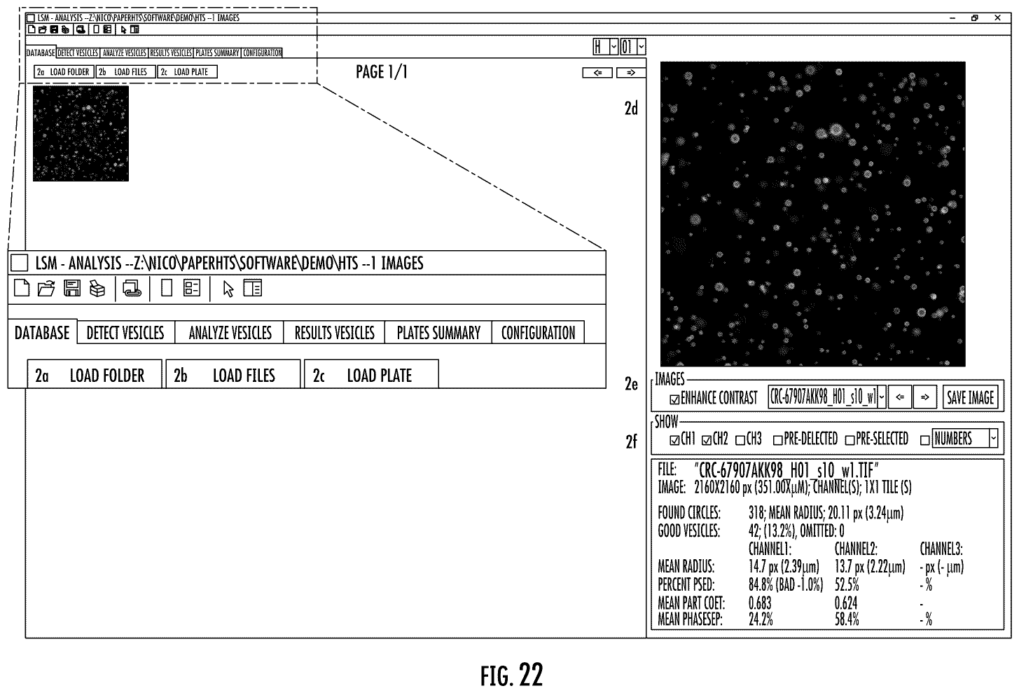

2. Database Tab
Using the “database” tab, images are loaded for analysis by selecting the “Load folder” (2a) or “Load files” (2b) buttons.
“Load plate” (2c) can be used to load stacked images, but requires a setting in the configuration tab, in particular the definition of free parameters in the image filenames, by regular expressions.
Once loaded, the image will be shown on the upper right hand corner of the window (2d).
The user can select which image is shown, and what information is shown on the image using the check boxes directly underneath the image (2e) in the stack (plate) modus. In the standard modus the user can click on the smaller thumbnail images on the left (in demo 1 and 2 there is only one image). The numbers in the information box also depend on the method chosen in the “Results vesicles” tab. For the demos they are fixed.
Loading large images for the very first time may require some time. Since thumbnails are created during the first loading, subsequent loading of those images will be much faster.
If the image was analyzed in a former session, some statistical information will be shown in the lower right corner of the window (2f). This was mainly used for software development purposes. See FIG. 22.

Referring to FIG. 22, a demo is shown. For demo1, select “Load Folder” (2a) and then select the “DemoSingleImage” folder. This dataset consists of a single image. For demo2, select “Load plate” (2c) and then select the “DemoStackImages” folder. This dataset comprises a single stack with 16 images. The first image is automatically previewed. Other images in the stack can be selected using the box in 2e. For demo3, select “Load plate” (2c) and choose the “DemoSetOfStacks” folder (not the timepoint_1). Stacks can be switched between by left clicking on the preview thumbnails in “database”. Other images in a stack can be selecting using the box in 2e.

3. Detect Vesicle Tab

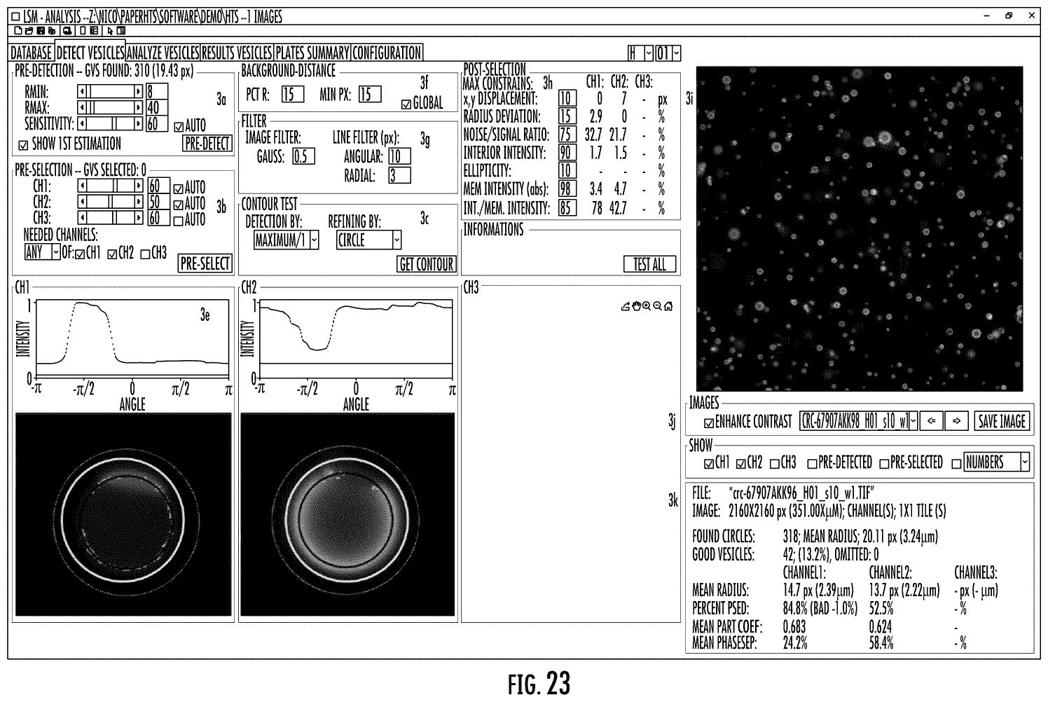

On the “Detect Vesicle” tab, parameters used to detect the vesicles can be optimized and the contour/membrane intensity profiles can be visualized. Reference is made to FIG. 23.
First, vesicles need to be detected using the “Pre-Detect” button (3a). This performs a Hough transform of the superpositioned green and red channels. (https://www.mathworks.com/help/images/ref/imfindcircles.html)
   1. Rₘᵢₙ and Rₘₐₓ (units of pixels) must be set manually according to the size of the vesicles in the image.
   2. The “auto” setting for sensitivity works for well in many instances and can be used in initial testing. This parameter can also be manually adjusted if desired, for example if not enough vesicles are detected.
The “Pre-Selection” function (3b) performs a Hough transform of the green channel and red channels individually.
1. Using the dropdown menu and checkboxes, this step enables the user to define whether the vesicles to be analyzed are required to contain signal in both the green and red channel (“all”) or any channel, i.e. green or red (“any”).
2. Pre-selection is not necessary for initial, simple tests, and can be skipped by selecting “none” in the dropdown menu under “needed channels”.
Next, the membrane/contours need to be identified (3c).
   1. As a first step, the “maximum” method should be selected, since it is much faster than the “fit” method. “/1/2 . . . ” indicate how many points/angles are skipped doing the detection, and are only important for the “fit” method.
   2. Left clicking on a pre-detected vesicle in the large image will start the contour detection process. In addition to thumbnail image of the chosen vesicle (3d), the results of the contour dection will be shown graphically for each channel (3e).
“Background distance” (3f) defines the distance from each vesicle at which background should be measured. The distance is defined either as terms of absolute number of pixels or as a percentage of the vesicle radius. Background can be measured locally for each vesicle, or globally as a mean value.
“Filter” (3g) can be used to perform two-dimensional image Gaussian filtering. The “line filter” option is applied to the contour (angular) and the radial intensities to find the contour position (radial).
“Constraints” (3h) can be applied to narrow the population of vesicles to be analyzed. This process plays a role in analyzing whole images or sets of images but not for single vesicles.
Following the predetection and preselection steps, the identified objects can be visualized in the image (3i) by choosing the corresponding checkbox (3j). The total number of vesicles detected and those remaining after preselection are shown in the bottom right panel (3k).
Referring FIG. 23, a Demo is shown (applies to demo 1, 2, and 3):
   If the image has not yet been fully analyzed, the first thing to do is click on the “Pre-detect” button (3a).
   Selecting the “Pre-detected” checkbox (3j) below the image provides a preview of all found circular objects as white circles in the image (3i)—(re)drawing the image will take longer.
   Left clicking on any detected (white) circle in the image runs the full contour/membrane detection.
   Clicking on “Pre-Select” (3b) runs the full contour detection for each detected circle. Results can be visualized by the “Pre-selected” checkbox (3j) below the image as red/green circles in the image (3i).
   In demo mode, all settings have been pre-set and cannot be changed.

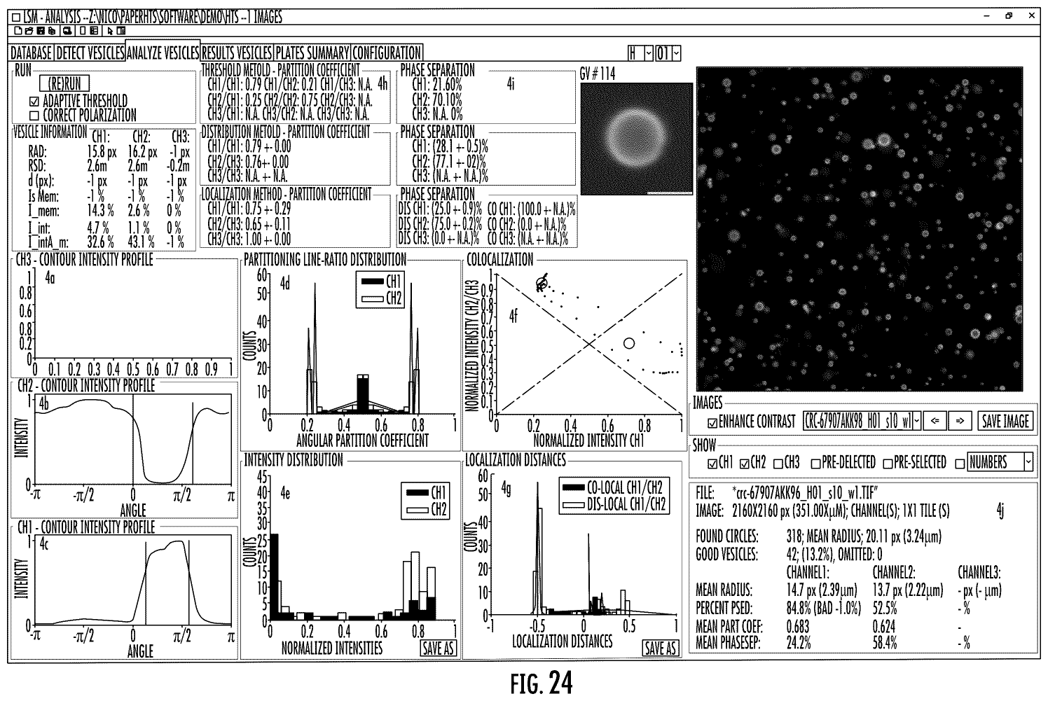

4. Analyze Vesicle Tab
On the “Analyze vesicle tab,” individual vesicles can be fully analyzed by left clicking on the vesicle in the large image on the right. The selected vesicle will be shown in the thumbnail image.
Results obtained from three different analysis methods (threshold, distribution, and colocalization) are shown for the chosen vesicle. They include:
   A graphical depiction of the fluorescence intensities in Ch1 and Ch2 for the chosen vesicle for the “threshold” method (4a, 4b, 4c), “distribution” method (4d, 4e) and “colocalization” method (4f, 4g).
      If desired, these graphs can be saved for individual vesicles.
      Note that only results obtained using the “distribution” and “colocalization” methods are used for data analysis as described elsewhere herein.
      Also note that even though the thumbnail image will update for all GPMVs when they are clicked on, the graphs will only be updated for GPMVs that pass all exclusion criteria are selected.
   The calculated partition coefficients (±SD) are also reported (4h).
      Ch1/Ch1 reports the partition coefficient for the fluorophore in Ch1 in the phase where it is brightest, i.e. Ch1/Ch1 reports the partition coefficient of fluorophore in Ch1 relative to the position of the enriched phase defined by Ch1.
         Ch2/Ch1 reports the partition coefficient for Ch1 in the phase where Ch2 is brightest (i.e. the opposite phase). See FIG. 24.
   Referring to FIG. 24, the percentage of the vesicle membrane (SD) that is enriched in red fluorescence (Ch1) or green fluorescence (Ch2) is also provided (4i).
An opportunity to save a summary of the numerical results in 4h and 4i for all of the vesicles in the image is provided on the next tab (“Results vesicles”).
The table in the lower right will automatically be updated with results for the vesicle population (4j).
It is not necessary to click the (Re)run button.
Referring to FIG. 24, a Demo is shown (applies to demo 1, 2, and 3)
   Left mouse click on a pre-detected vesicle will run the full analysis and will show all measurements and accompanying graphs obtained for all three analysis methods.

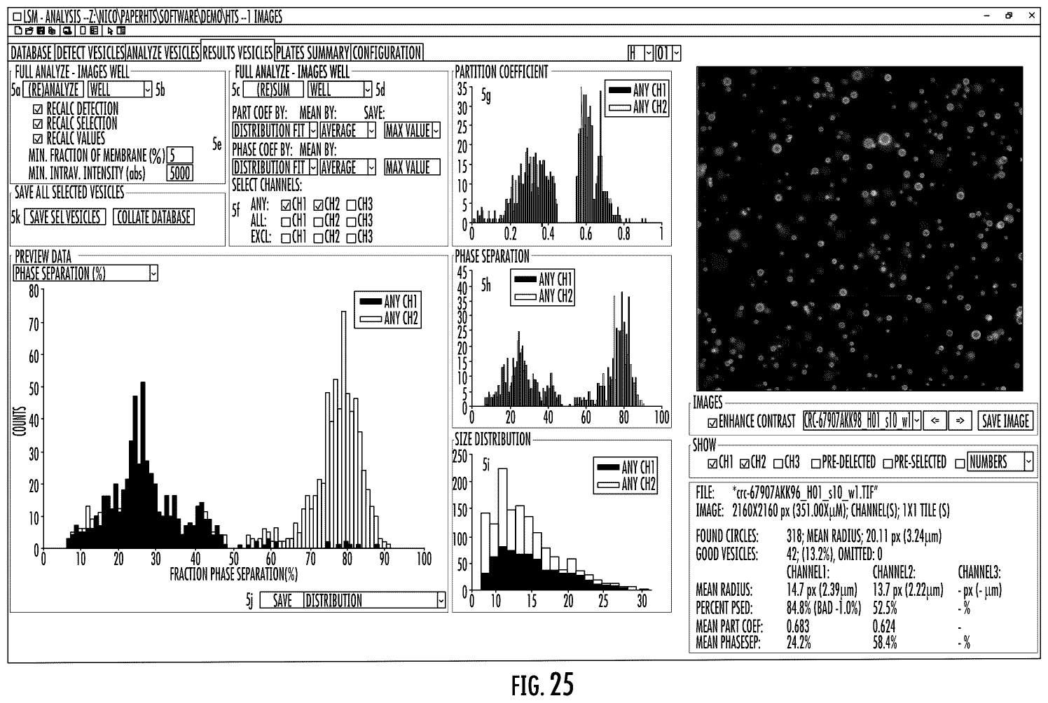

5. Results Vesicles Tab
Referring to FIG. 25, in the “Results vesicles” tab, a single image, “well” (stack of images) or “plate” (set of image stacks) can be automatically analyzed by clicking on “(Re) analyze” button (5a) after selecting the associated list entry (5b).

Since all data is saved during the analysis of an image, it is not necessary to repeat the detection, preselection, and analysis steps from the previous tabs.
Statistical analysis for the entire population of selected vesicles can be obtained by clicking on “(Re)Sum” button (5c) after choosing the associated list entry (5d), methods (5e) and channels of interest (5f). Graphs showing the results will then be shown. In this example, graphs showing the partition coefficients (5g) and percent phase separated vesicles (5h) are shown together with a plot of the size distribution of selected vesicles (5i). Results of the analysis can be saved as images or output the data in table format (5j).
It is also possible to output a table that provides the results for individual vesicles on a vesicle-by-vesicle basis by selecting “Say sel vesicles” (5k).
Referring to FIG. 25, additional demos are shown, as discussed as follows.
Demo 1:
   Choose “Image” in the drop down menu (5b) and then press the “(Re)Analyze” button (5a). This will start the full analysis using the settings defined on the previous tabs for the image. (Detection=>selection=>Analysis)
   To obtain statistics on all of the vesicles, press the “(Re)Sum” button (5c) after selecting “image” in the drop down menu next to that button (5d). This will sum up statistical information of all vesicles with the chosen analysis methods (threshold, distribution, or colocalization (5e). Previews of the data will be shown as graphs (5g-5i) which can be saved as images or in tabular format
Demo 2:
   Choose “Well” in the drop down menu (5b) and then press the “(Re)Anlyse” button (5a). This will start the full analysis with the settings defined on the previous tabs for the whole stack of images. Note: parallel computing will be used, and will require some time to be initiated for the first time the dataset is analyzed. The progress can be seen in the Matlab program.
   To obtain statistics on all of the vesicles, press the “(Re)Sum” together with “well” in the drop down menu next to that button (5d). This will sum up statistical information for all vesicles with the chosen analysis method. Previews of the data will be shown as graphs (5g-5j) which can be saved as images or in tabular format.
Both demos:
   “Save sel Vesicles” (5k) will save all information of the individual selected vesicles of the image or stack (well) of images.
   “Collate Database” (5k) will save the collated and statistically analyzed date of the image(s) in the database as a single table file. This yields a single number for each parameter, per image.
Demo 3:
   To fully analyze the whole dataset, choose “Plate” in the drop down menu (5b) next to the “(Re)Analyze” button (5a). The software will ask you to select the “plate” (set of stacks). Again choose “DemoSetOfStacks”, not “timepoint_1. This will start the full analysis with the settings defined on the previous tabs for the whole stack of images. (Note: This step has been already done and data is saved.)
   To preview statistics for single images (“image”) or a stack of images (“well”), press the “(Re)Sum” button (5c) and select the appropriate entry from the drop down menu (5d).

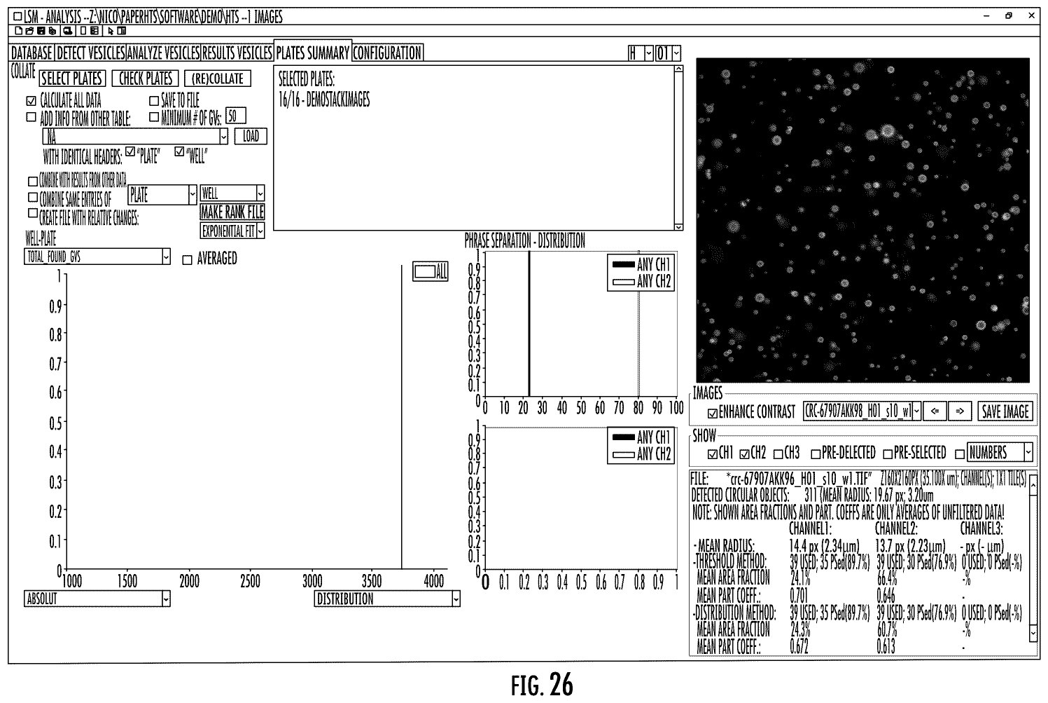

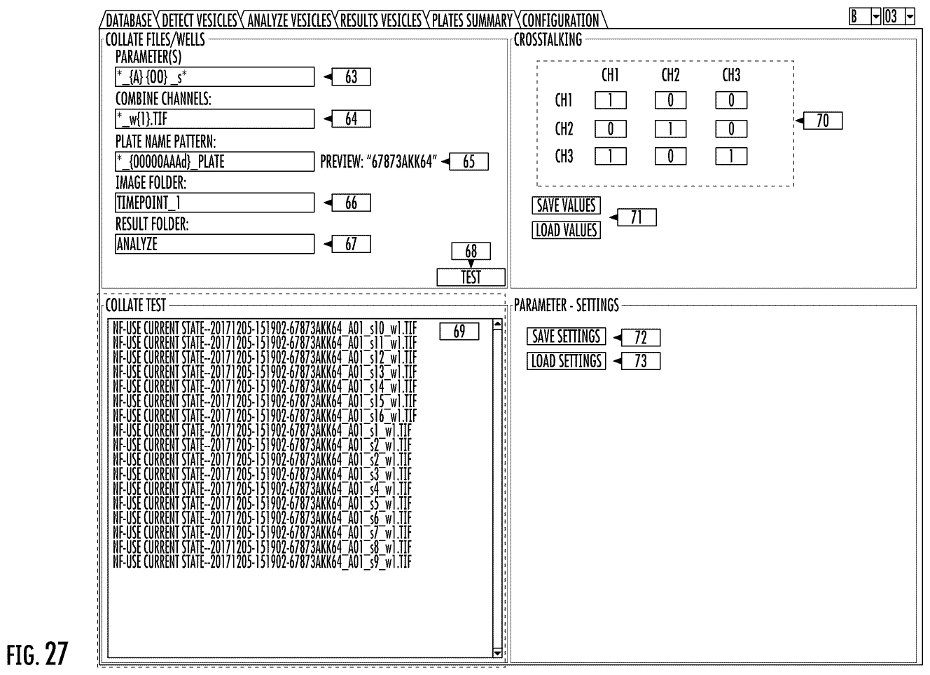

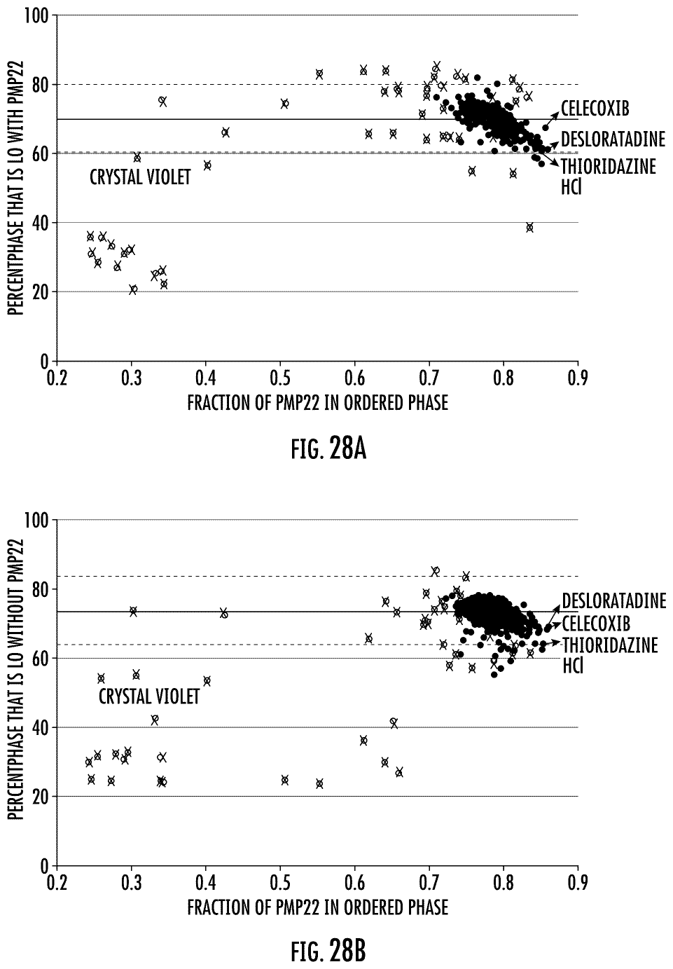

6. Plate Summary Tab
This tab collates and statistically analyzes sets of images stacks (for example as obtained for multiwell plates).
This information can combined with external information (such as concentration, temperature, drug treatment, etc.) in table format.
Because this step is designed to collate data across multiple stacks of images, a single image or single image stack will be represented as a single data point when displayed on this tab.
Referring to FIG. 26, additional demos are shown, as discussed as follows.
Demos (only applies to demo 3):
   This tab shows statistics for all of the stacks. The demo dataset consists of 12 stacks, thus 12 points will be included in the graphs.
   The plate to be examined is already selected and displayed in the “edit” window; there's no need to “select plates” again
   “Check Plates” button is redundant for the demo too.
   “(Re)Collate” button is necessary to show or update graphs.
   “Re-calculate all data” checkbox is only needed for the first time, or when methods from which to examine statistics are changed on the “Results Vesicles” tab.
   “Save to file” checkbox is only needed if data should be saved as a table.
   Further checkboxes are explained elsewhere and not required for the demo.

7. Configuration Tab

Referring to FIG. 27, the configuration tab can be used to set internal parameters used by VesA, such as the patterns to identify external parameters in the image filenames and the cross-talk matrix for the channels.

Save/Load Settings:

The “Save Settings” [72] and “Load Settings” [73] buttons can be used to save or load all values in the graphical interface. It might be useful to save the settings for a full analysis for each run. VesA also retains all settings when the program is closed and restores them when it is started again.

“Collate Files/Wells” Frame—Filename Patterns:

In the “Parameter(s)” box [63] a filename pattern can be set that identifies up to two free parameters used to identify a “well” belongings. Here, the free parameters are defined in curly brackets { }. The patterns are in the style of regular expressions:

Depending on whether the parameter are capital letters, small letters or numbers the notations are {A}, {a} or {0} with any repetitions, for example a three-digit number would be defined as {000}, or a combination of capital letters would be {AA}. A mix of numbers and letters as a parameter is not allowed (yet)! Also, the use of underscores or decimal points are not supported (yet)! The number of digits or letters defining the parameter (not the filename itself) must be the same for all images. (a variable length of numbers will be implemented soon).

An asterisk “*” represents any expression with a variable length. In case there is no asterisk at the beginning or end of the pattern, the filename must begin, or end respectively, with the given expression.

Examples:
“*_{A}{00}_s*”:
Would identify “crc-1234_B12_s_w1.png” as “B” for the first and “12” for the second parameter.
It would not identify “crc-1234_B1_s.png”, “crc-1234_d12_s.png”,
“crc-1234_CC12_s.png”
“T{000}C*.lsm”:
Would identify “T037CtestImage.lsm” or “T037C_testImage.lsm” as “037” for the first and only free parameter.
It would not identify “ImageT037Ctest_Image.lsm”, or “T37CtestImage.lsm”, or “T037CtestImage.png”, or “T37_0CtestImage.lsm”

In the “Combine Channels” box [64] a pattern for filenames to combine channel separated image files can be defined. It follows similar rules as for the “Parameter(s)” patterns.

Examples:
“*_w{1}.tif”:
Would identify file “crc-1234_B12_s_w1.png” as channel 1 and “crc-1234_B12_s_w2.png” as channel 2.
It would not identify “crc-1234_B12_s_w01.png”, or “crc-1234_B12_s_w1_a.png”

In the “Plate name pattern” box [65] an extraction of a certain plate name pattern of the folder containing the plate/images can be defined. Next to that box preview will be shown after the “test” button [68] is used.

Here, no asterisks “*” are allowed (yet)!

The content in the curly brackets { } define the plate name. It can be any combination of capital letters “A”, small letters “a”, numbers with of defined digits “0” or numbers with a variable length “d”.

Examples:
“−{00000AAAd}_plate”:
Would set “12345ABC15” as the plate name of folder “test-12345ABC15_plate12ab” or
“12345CBA1” of folder “test-12345CBA1_plate”.
It would not identify “test-1234ABC15_plate12ab”, “test_12345ABC15_plate12ab”, or “test-12345ABC15_images”

If the box is left blank, or the pattern could not be found in the folder name the whole folder name will considered as a plate name.

The “Image folder” [66] and “Result folder” [67] subfolders can be defined where VesA is looking for images and saves all internal tables, respectively. In case the result folder does not exist, it will be generated automatically. Both boxes can be kept empty. In that case VesA won't look for image subfolders and will save the tables aside the image files.

8. Troubleshooting

If the demo datasets do not yield the expected results:
0. Manually input the settings.
   Or
1. Manually load the settings.
   Go to the “configuration” tab.
   Click on “load settings” (Mid bottom)
   Load “DemoSettings.mat” in the “presets” folder.

Output Csv File Headers

Note: when running in Free mode selecting “Any” in the Results Vesicles tab will result in data that are calculated from vesicles that have signal in any of the selected channels (i.e. they could contain red signal OR green signal). These files headers will contain “Ch1 w/Ch1” or “Ch2 w/Ch2”. Selecting “All” will refine the results and provide data from vesicles which have all of the selected channels (i.e., only vesicles that are both green and red will be included in the analysis). These file headers will contain “w/all channels”.

| First Row Header | Second Row Header | Explanation |
|---|---|---|
| | Plate | Plate name from input files selected in |
| | | Database tab |
| | Well | Alpha-numeric well designations |
| Vesicle detection | Nr of found vesicles | Total number of vesicles detected |
| Vesicle detection | Nr of used vesicles w/all | Total number of vesicles that are used for |
| | channels | measuring membrane partition function (see |
| | | note above for Any vs. All Channels) |
| Radial max; | Mean radius of all | Mean radius of the vesicles in pixels |
| Circular refine | channels (px) | |
| Radial max; | Mean radius of all | Standard deviation of above |
| Circular refine | channels STD (px) | |
| Vesicle detection | Nr of used vesicles w/all | Total number of vesicles that are used for |
| | channels | measuring membrane partition function |
| Radial max; | Mean radius of all | Mean radius of the vesicles (repeat) |
| Circular refine | channels (px) | |
| Radial max; | Mean radius of all | Standard deviation of above (repeat) |
| Circular refine | channels STD (px) | |
| Vesicle detection | Nr of used vesicles w/all | Total number of vesicles that are used for |
| | channels | measuring membrane partition function |
| | Nr of phase separated | Number of vesicles that are phase separated |
| | vesicles Ch1 w/all | |
| | channels | |
| Distribution | Fraction of phase | Number of vesicles that are phase |
| method w/max val. | separated vesicles Ch1 | separated/Total number of used vesicles |
| of double Gauss fit | (%) | |
| Distribution | Mean Phase Sep. of | Average percentage of membrane in phase |
| method w/max val. | separated vesicles Ch1 | separated vesicle that is covered in a lipid |
| of double Gauss fit | w/all channels (%) | preference |
| Distribution | Mean Phase Sep. of | Std Dev of above |
| method w/max val. | separated vesicles Ch1 | |
| of double Gauss fit | w/all channels STD (%) | |
| Distribution | Mean Phase Sep. of all | Average percentage of membrane in all |
| method w/max val. | vesicles Ch1 w/all | vesicles that is covered in a lipid preference |
| of double Gauss fit | channels (%) | |
| Distribution | Mean Phase Sep. of all | Std Dev of above |
| method w/max val. | vesicles Ch1 w/all | |
| of double Gauss fit | channels STD (%) | |
| | Nr of phase separated | Number of vesicles that are phase separated |
| | vesicles Ch2 w/all | using marker 2 |
| | channels | |
| Distribution | Fraction of phase | Fraction phase separated vesicle |
| method w/max val. | separated vesicles Ch2 | |
| of double Gauss fit | (%) | |
| Distribution | Mean Phase Sep. of | Average percentage of membrane in phase |
| method w/max val. | separated vesicles Ch2 | separated vesicle that is covered |
| of double Gauss fit | w/all channels (%) | by marker 2 |
| Distribution | Mean Phase Sep. of | Std Dev of above |
| method w/max val. | separated vesicles Ch2 | |
| of double Gauss fit | w/all channels STD (%) | |
| Distribution | Mean Phase Sep. of all | Average percentage of membrane in all |
| method w/max val. | vesicles Ch2 w/all | vesicles that is covered by marker 2 |
| of double Gauss fit | channels (%) | |
| Distribution | Mean Phase Sep. of all | Std Dev of above |
| method w/max val. | vesicles Ch2 w/all | |
| of double Gauss fit | channels STD (%) | |
| Distribution | Partition coefficient of | Partition coefficient of channel 1 marker in |
| method w/max val. | Ch1 w/all channels | channel 2 (Ch1/Ch2) from distribution |
| of single Gauss fit | | method = 1 − Part. Coeff. of Ch1/Ch1 |
| | | (as reported in Analyze Vesicles tab) |
| Distribution | Partition coefficient of | Std Dev of above |
| method w/max val. | Ch1 w/all channels STD | |
| of single Gauss fit | | |
| Distribution | Partition coefficient of | Partition coefficient of channel 2 marker in |
| method w/max val. | Ch2 w/all channels | channel 2 (Ch2/Ch2) from distribution |
| of single Gauss fit | | method = Part. Coeff. of Ch2/Ch2 |
| | | (as reported in Analyze Vesicles tab) |
| Distribution | Partition coefficient of | Std Dev of above |
| method w/max val. | Ch2 w/all channels STD | |
| of single Gauss fit | | |
| Contour detection | Noise/Signal Ch1 w/all | Signal to noise ratios average |
| | channels (%) | for marker 1 |
| Contour detection | Noise/Signal Ch1 w/all | Std Dev of above |
| | channels STD (%) | |
| Contour detection | Intravesicular Intensity | What is the fluorescence intensity |
| | Ch1 w/all channels (abs) | inside vesicle using marker 1 |
| Contour detection | Intravesicular Intensity | Std Dev of above |
| | Ch1 w/all channels STD | |
| | (abs) | |
| Radial fits | Detected fraction of | Empty |
| | membrane Ch1/w all | |
| | channels (%) | |
| Radial fits | Detected fraction of | Empty |
| | membrane Ch1/w all | |
| | channels STD (%) | |
| Contour detection | Membrane intensity Ch1/w | Average Membrane intensity defining |
| | all channels (%) | cutoff for profile using marker 1 |
| Contour detection | Membrane intensity Ch1/w | Std Dev of above |
| | all channels STD (%) | |
| Radial fits | Membrane thickness Ch1/w | Empty |
| | all channels (px) | |
| Radial fits | Membrane thickness Ch1/w | Empty |
| | all channels STD (px) | |
| Contour detection | Intravesicular/Membrane | Empty |
| | intensity Ch1/w all | |
| | channels (%) | |
| Contour detection | Intravesicular/Membrane | Empty |
| | intensity Ch1/w all | |
| | channels STD (%) | |
| Contour detection | Noise/Signal Ch2 w/all | Signal to noise ratios average |
| | channels (%) | for marker 2 |
| Contour detection | Noise/Signal Ch2 w/all | Std Dev of above |
| | channels STD (%) | |
| Contour detection | Intravesicular Intensity | Internal fluorescence intensity |
| | Ch2 w/all channels (abs) | inside vesicle using marker 2 |
| Contour detection | Intravesicular Intensity | Std Dev of above |
| | Ch2 w/all channels STD | |
| | (abs) | |
| Radial fits | Detected fraction of | Empty |
| | membrane Ch2/w all | |
| | channels (%) | |
| Radial fits | Detected fraction of | Empty |
| | membrane Ch2/w all | |
| | channels STD (%) | |
| Contour detection | Membrane intensity Ch2/w | Average Membrane intensity defining |
| | all channels (%) | cutoff for profile using marker 2 |
| Contour detection | Membrane intensity Ch2/w | Std Dev of above |
| | all channels STD (%) | |
| Radial fits | Membrane thickness Ch2/w | Empty |
| | all channels (px) | |
| Radial fits | Membrane thickness Ch2/w | Empty |
| | all channels STD (px) | |
| Contour detection | Intravesicular/Membrane | Empty |
| | intensity Ch2/w all | |
| | channels (%) | |
| Contour detection | Intravesicular/Membrane | Empty |
| | intensity Ch2/w all | |
| | channels STD (%) | |
| | PIRow | Well letter - useful for sorting data |
| | PICol | Column number - useful for sorting data |

### Example 6

### Preliminary HTS Screen for Compounds that Alter the Phase Partitioning of Membrane Proteins in GPMVs

This EXAMPLE pertains to the use of the presently disclosed screening approach to conduct HTS for compounds that alter the native population distribution of integral membrane proteins between location in raft phase membrane domains versus location in disordered phase membrane domains. This EXAMPLE employs as a test case the human membrane protein known as peripheral myelin protein 22 (PMP22). It was shown that under native-like conditions in human cell-derived GPMVs, PMP22 prefers to partition into raft phase membrane domains relative to disordered phase domains by a ratio 4-to-1 (Marinko et al. (2020) Peripheral myelin protein 22 preferentially partitions into ordered phase membrane domains. Proc Natl Acad Sci USA 117(25):14168-14177). Another way of expressing this result is to say that the partitioning coefficient for PMP22 in favor of the raft phase is roughly 0.80. The fact that PMP22 exhibits a pronounced but not absolute phase domain preference in GPMVs makes it a suitable candidate to test if small molecules can be discovered that alter its phase preference in either direction, favoring a shift toward either the disordered phase or further in favor of the ordered phase.

In some embodiments, one or more membrane proteins is labeled with one or more fluorescent labels, each of which could be a fused fluorescent protein, a chemically-reacted fluorescent compound, or an unnatural amino acid that either is fluorescent or can be reacted with a fluorescent compound. The one or more proteins are then detected, such as with fluorescence microscopy, and quantitated in each phase using the software. By way of example, PMP22 containing a MYC epitope sequence can be employed in a vesicle such as a GPMV, such as by expressing a MYC-Tagged PMP22 in Hela cells, followed by use of a commercial fluorescently labeled anti-MYC antibody to confer detectable fluorescence to PMP22 in GPMVs. Indeed, the fluorescent labels come in various colors, which means that one can detect several differently labeled molecules at the same time in the assay, such as by using a microscope. In some embodiments, for example, where one uses a label that marks the non-raft phase domain in red, one can label a membrane protein (like PMP22) in green. By visualizing them using fluorescence microscopy, one can then detect whether the PMP22 is in the raft or non-raft phase domain, and to what extent. By way of a particular example, PMP22 prefers to localize within the raft phase domain, as evidenced by a finding that it is mostly excluded from the non-raft phase. There are some proteins, on the other hand, that exhibit a preference not to reside in lipid raft phase domains, such as a protein known as C99 (see Capone et al., 2021) If one labels the C99 green and again uses a red non-raft marker, in that case both colors show up in the same phase. Note that the convention here is that when they overlap they appear yellow in the merged image. In some embodiments, the presently disclosed methods and systems seek to identify compounds that change the “affinity” of a particular protein for the raft vs. non-raft phase domain. In some embodiments, this is read out as a change in relative brightness of the fluorescence signal associated with that protein in one phase versus the other.

By way of example and not limitation, in some embodiments PMP22 containing a MYC epitope sequence was employed in the GPMVs (hereafter referred to as “PMP22”, such as by expressing a MYC-Tagged PMP22 in Hela cells, followed by formation of PMP22 containing GPMVs, followed by addition of a commercial fluorescently labeled anti-MYC antibody to confer fluorescence to PMP22 in GPMVs, thereby making PMP22 detectable to enable its quantitation in the raft phase domains versus in the non-raft phase domains. See Marinko et al. (2020) Proc Natl Acad Sci USA 117(25):14168-14177.

The assay was scaled up to carry out high throughput screens (HTS). The strategy for these screens is similar to those outlined for our approach to finding compounds that shift the raft vs. non-raft phase domain distribution in GPMVs, except that in this case we were interested in discovering compounds that either shift PMP22 out of rafts or enhance its partitioning into the ordered raft phase domain. This pilot HTS screen involved the 1184 compound library of FDA-approved drugs at a concentration of 10 micromolar each and was carried out in the Vanderbilt HTS Core. We predicted that select compounds might sometimes change not only the phase partitioning of PMP22, but also change the actual amounts of ordered (raft) versus disordered phase domains that are present. We also wondered whether PMP22 and certain compounds might act synergistically (through any one of several possible mechanisms) to alter membrane phase behavior. This could potentially cause the compounds to have different effects on the ratio of raft versus disordered domains in the absence relative to the presence of the PMP22 protein. To test these possibilities, we compared the results of our screen to the results of preliminary screens carried out on the same compounds in GPMVs lacking expressed PMP22. This allowed us to directly evaluate the effects of the compounds under both conditions. The plots shown in FIGS. 28A and 28B, as well as additional data not shown reveal several classes of PMP22-impacting hits from this 1184 compound library:

Class I: These compounds significantly altered the phase domain partitioning of PMP22, but had no effect on the relative proportions of raft versus disordered domains.

Class II: These compounds significantly altered both the phase domain partitioning of PMP22, but do not alter the relative proportions of raft versus disordered phase domains when PMP22 is absent.

Class III: These compounds significantly altered both the phase domain partitioning of PMP22 and the relative proportions of raft versus disordered phase domains, either in the absence or presence of PMP22.

Class IV: These compounds have no effect on the phase partitioning of PMP22, but change the relative populations of the ordered versus disordered phase domains in a PMP22-dependent manner.

False positives: These compounds are not bona fide hits and can be identified by routine visual inspection of the HTS fluorescence microscope images for compounds deemed by the automated software to be hits. They were found to derive from one of three phenomena: (A) PMP22 appears as concentrated puncta in the GPMVs. (B) Widespread clustering of GPMVs. (C) GPMVs are present, but few phase-separated domains are observed.

Class I, Class II, Class III, and Class IV compounds are all of potential interest as tool compounds or potential drug molecules and represent distinct classes of deliverables generated by this approach. False positive compounds may also be of interest and are readily detected and classified as such by our screening/imaging/analysis approach.

As illustrated in FIGS. 28A and 28B, the preliminary FDA drug-screen indicated that virtually all of the candidate Class I hits that initially appeared to shift PMP22 out of the ordered phase and toward the disordered phase turned out to be false-positives. However, there were a handful of true Class I compounds that reproducibly shifted the phase preference of PMP22 in favor of the ordered (raft) phase by statistically significant amounts (on the order of 10%) without significantly perturbing the relative ordered (raft) vs. disordered phase proportions either in the absence or presence of PMP22. These include Desloratadine, Celecoxib, and Thioridazine-HCl (see FIGS. 28A and 28B). The increased raft partitioning coefficient seen for PMP22 in the presence of each compound was reproducible, even when the concentration of each was lowered from 10 to 1 micromolar.

The data shown in FIGS. 28A and 28B also show that some compounds do not significantly alter the phase partitioning of PMP22, but exhibit Class IV activities by altering partitioning of the GPMV membrane into raft versus non-raft phase domains in manner that depends on whether or not PMP22 was present in the GPMVs.

Given the small size of the library these results are encouraging and demonstrate proof of principle and reduction to practice.

These results provide proof of principle that the presently disclosed approaches can be used to discover compounds that alter the raft vs. disordered phase partitioning of membrane proteins.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
Adachi et al. (2007) The inhibitory effect of (−)-epigallocatechin gallate on activation of the epidermal growth factor receptor is associated with altered lipid order in HT29 colon cancer cells. Cancer Res 67(13):6493-501.
Baell & Holloway (2010) New substructure filters for removal of pan assay interference compounds (PAINS) from screening libraries and for their exclusion in bioassays. J Med Chem 53(7):2719-40.
Baumgart et al. (2007) Large-scale fluid/fluid phase separation of proteins and lipids in giant plasma membrane vesicles. Proc Natl Acad Sci USA 104(9):3165-70.
Bernardes & Fialho (2018) Perturbing the dynamics and organization of cell membrane components: A new paradigm for cancer-targeted therapies. Int J Mol Sci 19(12):3871.
Bukrinsky et al. (2020) Lipid rafts and pathogens: The art of deception and exploitation. J Lipid Res 61(5):601-610.
Capone et al. (2021) The C99 domain of the amyloid precursor protein resides in the disordered membrane phase. Journal of Biological Chemistry 296:100652.
Castello-Serrano, I., et al. (2020) Myelin-Associated MAL and PLP Are Unusual among Multipass Transmembrane Proteins in Preferring Ordered Membrane Domains. J Phys Chem B 124, 5930-5939
Chiantia et al. (2007) Raft domain reorganization driven by short- and long-chain ceramide: a combined AFM and FCS study. Langmuir 23:7659-7665.
Chichili & Rodgers (2009) Cytoskeleton-membrane interactions in membrane raft structure. Cell Mol Life Sci 66(14):2319-28.
Chinnapen et al. (2012) Lipid sorting by ceramide structure from plasma membrane to ER for the cholera toxin receptor ganglioside GM1. Dev Cell 23:573-586.
Cornell et al. (2017) n-Alcohol length governs shift in Lo-Ld mixing temperatures in synthetic and cell-derived membranes. Biophys J 113:1200-1211.
Drummen et al. (2002) C11-BODIPY(581/591), an oxidation-sensitive fluorescent lipid peroxidation probe: (micro)spectroscopic characterization and validation of methodology. Free Radic Biol Med 33:473-490.
Duran et al. (2012) Sphingomyelin organization is required for vesicle biogenesis at the Golgi complex. EMBO J 31(24):4535-46.
Endapally et al. (2019) Molecular Discrimination between Two Conformations of Sphingomyelin in Plasma Membranes. Cell 176:1040-1053 e1017.
Gerstle et al. (2018) Giant plasma membrane vesicles: An experimental tool for probing the effects of drugs and other conditions on membrane domain stability. Methods Enzymol 603:129-150.
Gimpl & Gehrig-Burger (2011) Probes for studying cholesterol binding and cell biology. Steroids 76:216-231.
Gray et al. (2013) Liquid general anesthetics lower critical temperatures in plasma membrane vesicles. Biophys J 105(12):2751-9.
Heczkova & Slotte (2006) Effect of anti-tumor ether lipids on ordered domains in model membranes. FEBS Lett 580(10):2471-6.
Holowka et al. (2018) Short chain ceramides disrupt immunoreceptor signaling by inhibiting segregation of Lo from Ld Plasma membrane components. Biol Open 7(9):bio034702.
Honigmann et al. (2014) A lipid bound actin meshwork organizes liquid phase separation in model membranes. Elife 3:e01671.
Hryniewicz-Jankowska et al. (2014) Membrane rafts as a novel target in cancer therapy. Biochim Biophys Acta 1845(2):155-65.
Inglese et al. (2007) High-throughput screening assays for the identification of chemical probes. Nat Chem Biol 3(8):466-79.
Ingolfsson et al. (2014) Phytochemicals perturb membranes and promiscuously alter protein function. ACS Chem Biol 9(8):1788-98.
Jin et al. (2006) Characterization and application of a new optical probe for membrane lipid domains. Biophys J 90(7):2563-75.
Johnson et al. (2010) Temperature-dependent phase behavior and protein partitioning in giant plasma membrane vesicles. Biochim Biophys Acta 1798(7):1427-35.
Johnson et al. (2017) Mechanistic Insights into the Cholesterol-dependent Binding of Perfringolysin O-based Probes and Cell Membranes. Sci Rep 7:13793.
Kaiser et al. (2009) Order of lipid phases in model and plasma membranes. Proc Natl Acad Sci USA 106:16645-16650.
King et al. (2020) ER membranes exhibit phase behavior at sites of organelle contact. Proc Natl Acad Sci USA 117:7225-7235.
Kusumi, A., et al. (2020) Defining raft domains in the plasma membrane. Traffic 21, 106-137 Levental & Levental (2015) Giant plasma membrane vesicles: Models for understanding membrane organization. Curr Top Membr 75:25-57.
Levental et al. (2011) Raft domains of variable properties and compositions in plasma membrane vesicles. Proc Natl Acad Sci USA 108(28):11411-6.
Levental et al. (2016) Polyunsaturated lipids regulate membrane domain stability by tuning membrane order. Biophys J 110(8):1800-10.
Levental et al. (2020a) Lipid rafts: Controversies resolved, mysteries remain. Trends Cell Biol 30(5):341-353.
Levental et al. (2020b) Lipidomic and biophysical homeostasis of mammalian membranes counteracts dietary lipid perturbations to maintain cellular fitness. Nat Commun 11(1):1339.
Lietha & Izard (2020) Roles of membrane domains in integrin-mediated cell adhesion. Int J Mol Sci 21(15):5531.
Lingwood, D., et al., (2008) Plasma membranes are poised for activation of raft phase coalescence at physiological temperature. Proc Natl Acad Sci USA 105, 10005-10010
Lorent et al. (2017) Structural determinants and functional consequences of protein affinity for membrane rafts. Nat Commun 8(1):1219.
Lorent et al. (2020) Plasma membranes are asymmetric in lipid unsaturation, packing and protein shape. Nat Chem Biol 16(6):644-652.
Machta et al. (2016) Conditions that stabilize membrane domains also antagonize n-alcohol anesthesia. Biophys J 111(3):537-545.
Miller et al. (2020) Divide and conquer: How phase separation contributes to lateral transport and organization of membrane proteins and lipids. Chem Phys Lipids 233:104985.
Neves et al. (2016a) Resveratrol induces ordered domains formation in biomembranes: Implication for its pleiotropic action. Biochim Biophys Acta 1858(1):12-8.
Neves et al. (2016b) Resveratrol interaction with lipid bilayers: a synchrotron X-ray scattering study. Langmuir 32(48):12914-12922.
Owen et al. (2006) Fluorescence lifetime imaging provides enhanced contrast when imaging the phase-sensitive dye di-4-ANEPPDHQ in model membranes and live cells. Biophys J 90(11):L80-2.
Podkalicka et al. (2015) MPP1 as a factor regulating phase separation in giant plasma membrane-derived vesicles. Biophys J 108(9):2201-11.
Pyenta et al. (2001) Cross-correlation analysis of inner-leaflet-anchored green fluorescent protein co-redistributed with IgE receptors and outer leaflet lipid raft components. Biophys J 80:2120-2132.
Raghunathan et al. (2015) Membrane transition temperature determines cisplatin response. PLoS One 10(10):e0140925.
Raghunathan et al. (2016) Glycolipid crosslinking is required for cholera toxin to partition into and stabilize ordered domains. Biophys J 111:2547-2550.
Rayermann et al. (2017) Hallmarks of reversible separation of living, unperturbed cell membranes into two liquid phases. Biophys J 113:2425-2432.
Sezgin et al. (2012a) Elucidating membrane structure and protein behavior using giant plasma membrane vesicles. Nat Protoc 7(6):1042-51.
Sezgin et al. (2012b) Partitioning, diffusion, and ligand binding of raft lipid analogs in model and cellular plasma membranes. Biochim Biophys Acta 1818(7):1777-84.
Sezgin et al. (2014) Measuring lipid packing of model and cellular membranes with environment sensitive probes. Langmuir 30(27):8160-6.
Sezgin et al. (2015) Adaptive lipid packing and bioactivity in membrane domains. PLoS One 10:e0123930.
Sezgin et al. (2016) A comparative study on fluorescent cholesterol analogs as versatile cellular reporters. J Lipid Res 57:299-309.
Sezgin et al. (2017) The mystery of membrane organization: Composition, regulation and roles of lipid rafts. Nat Rev Mol Cell Biol 18(6):361-374.
Steinkuhler et al. (2019) Mechanical properties of plasma membrane vesicles correlate with lipid order, viscosity and cell density. Commun Biol 2:337.
Stone et al. (2017) Protein sorting by lipid phase-like domains supports emergent signaling function in B lymphocyte plasma membranes. Elife 6:e19891.
Sviridov et al. (2020) Lipid rafts as a therapeutic target. J Lipid Res 61(5):687-695.
Sych et al. (2021) How Does Liquid-Liquid Phase Separation in Model Membranes Reflect Cell Membrane Heterogeneity? Membranes (Basel) 11:323.
Toulmay & Prinz (2013) Direct imaging reveals stable, micrometer-scale lipid domains that segregate proteins in live cells. J Cell Biol 202:35-44.
Tsubone et al. (2019) Contrasting roles of oxidized lipids in modulating membrane microdomains. Biochim Biophys Acta Biomembr 1861:660-669.
Tsuchiya & Mizogami (2020) Interaction of drugs with lipid raft membrane domains as a possible target. Drug Target Insights 14:34-47.
Veatch & Keller (2003) A closer look at the canonical ‘Raft Mixture’ in model membrane studies. Biophys J 84:725-726.
Veatch et al. (2008) Critical fluctuations in plasma membrane vesicles. ACS Chem Biol 3(5):287-93.
Verma (2009) HIV: A raft-targeting approach for prevention and therapy using plant-derived compounds (review). Curr Drug Targets 10(1):51-9.
Verma et al. (2018) Host lipid rafts play a major role in binding and endocytosis of Influenza A virus. Viruses 10(11):650.
Wesolowska et al. (2009) Giant unilamellar vesicles—a perfect tool to visualize phase separation and lipid rafts in model systems. Acta Biochim Pol 56:33-39.
Zaremberg et al. (2020) Lipids and membrane microdomains: The glycerolipid and alkylphosphocholine class of cancer chemotherapeutic drugs. Handb Exp Pharmacol 259:261-288.
Zhang (2007) A pair of new statistical parameters for quality control in RNA interference high-throughput screening assays. Genomics 89(4):552-61.
Zhang (2011) Illustration of SSMD, z score, SSMD*, z* score, and t statistic for hit selection in RNAi high-throughput screens. J Biomol Screen 16(7):775-85.
Zhang et al. (1999) A simple statistical parameter for use in evaluation and validation of high throughput screening assays. J Biomol Screen 4(2):67-73.
Zhao & Zhang (2020) Phase Separation in Membrane Biology: The Interplay between Membrane-Bound Organelles and Membraneless Condensates. Dev Cell 55:30-44.
Zhou et al. (2012) Nonsteroidal anti-inflammatory drugs alter the spatiotemporal organization of Ras proteins on the plasma membrane. J Biol Chem 287(20):16586-16595.
Zhou et al. (2013) Bile acids modulate signaling by functional perturbation of plasma membrane domains. J Biol Chem 288(50):35660-70.
Zidovetzki & Levitan (2007) Use of cyclodextrins to manipulate plasma membrane cholesterol content: Evidence, misconceptions and control strategies. Biochim Biophys Acta 1768(6):1311-24.
Veatch, S. L., and Keller, S. L. (2003) A closer look at the canonical ‘Raft Mixture’ in model membrane studies. Biophys J 84, 725-726
Wesolowska, O., et al. (2009) Giant unilamellar vesicles—a perfect tool to visualize phase separation and lipid rafts in model systems. Acta Biochim Pol 56, 33-39
Sych, T., et al. (2021) How Does Liquid-Liquid Phase Separation in Model Membranes Reflect Cell Membrane Heterogeneity? Membranes (Basel) 11
King, C., et al. (2020) ER membranes exhibit phase behavior at sites of organelle contact. Proc Natl Acad Sci USA 117, 7225-7235
Zhao, Y. G., and Zhang, H. (2020) Phase Separation in Membrane Biology: The Interplay between Membrane-Bound Organelles and Membraneless Condensates. Dev Cell 55, 30-44
Toulmay, A., and Prinz, W. A. (2013) Direct imaging reveals stable, micrometer-scale lipid domains that segregate proteins in live cells. J Cell Biol 202, 35-44

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. An automated method for identifying a compound that impacts a characteristic of a lipid raft phase domain, a characteristic of a non-raft phase domain, and/or a characteristic of one or more membrane proteins, the method comprising:
contacting a population of vesicles, wherein one or more vesicles in the population of vesicles optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are phase separated;
detecting a signal from the population of vesicles; and
identifying the candidate compound as having an impact on a characteristic of a lipid raft phase domain, a characteristic of a non-raft phase domain, and/or a characteristic of one or more membrane proteins based on the signal.

2. The method of claim 1, wherein the signal is used to assess whether the candidate compound induces changes in the percent of vesicles containing the single visible phase within the population of vesicles; changes in the relative sizes and/or characteristics of the lipid raft phase domain versus the non-raft phase domain; and/or changes in the distribution of one or more membrane proteins between the lipid raft phase and the non-raft phase domains.

3. The method of claim 1, wherein the characteristic of a lipid raft phase domain, a characteristic of a non-raft phase domain, and/or a characteristic of one or more membrane proteins comprises membrane phase behavior, partitioning of proteins between the lipid raft phase domain and the non-raft phase domain, or both.

4. The method of claim 1, wherein the vesicle comprises a Giant Plasma-Membrane Derived Vesicle (GPMV).

5. The method of claim 1, wherein the lipid raft phase domain, the non-raft phase domain, and/or the one or more membrane proteins are distinguishably labeled.

6. The method of claim 5, wherein (a) the lipid raft phase domain is labeled with a first fluorescent label and the non-raft phase domain is labeled with a second fluorescent label, wherein the first fluorescent label and the second fluorescent label are distinguishable from each other; (b) the lipid raft phase domain is labeled with a first fluorescent label and the one or more membrane proteins is labeled with a second fluorescent label, wherein the first fluorescent label and the second fluorescent label are distinguishable from each other; (c) the non-raft phase domain is labeled with a first fluorescent label and the one or more membrane proteins is labeled with one or more additional fluorescent labels, wherein the first fluorescent label and the additional fluorescent labels are all distinguishable from each other; and (d) the lipid raft phase domain is labeled with a first fluorescent label, the non-raft phase domain is labeled with a second fluorescent label, and the one or more membrane proteins is labeled with a third or more fluorescent label, wherein the first fluorescent label, the second fluorescent label, and the third or more fluorescent labels are all distinguishable from each other.

7. The method of claim 1, wherein detecting a signal in the population of vesicles further comprises detecting a detectable label in the population of vesicles, taking one or more images of one or more vesicles in the population of vesicles, or a combination thereof.

8. The method of claim 1, wherein detecting a signal from the population of vesicles comprises identifying one or more vesicles, excluding one or more vesicles that do not meet one or more selection criteria, mapping a position of membrane contour for each of the one or more vesicles, generating a plot of fluorescence intensity along the membrane of the vesicle, calculating a partition coefficient of a vesicle, scoring a vesicle as phase separated or not, or any combination of any of the foregoing.

9. The method of claim 8, wherein calculating a partition coefficient of a vesicle comprises calculating the partition coefficients of the fluorescent labels and/or fluorescently labeled membrane proteins as a function of angle around the entire vesicle.

10. The method of claim 8, wherein scoring a vesicle as phase separated or not comprises determining whether a histogram of the angular partition coefficient of a vesicle comprises a single peak centered around 0.5 or instead contains multiple peaks reflecting a presence of fluorophore-rich and fluorophore-poor domains.

11. The method of claim 8, wherein scoring a vesicle as phase separated or not comprises separating data points into two separate quadrants for vesicles having phase separated membranes and colocalizing datapoints on a diagonal for membranes containing a single uniform phase.

12. The method of claim 1, further comprising correcting the imaging for a position effect.

13. The method of claim 1, wherein identifying the candidate compound as having an impact on a characteristic of a lipid raft based on the signal comprises detecting a change in phase separation in one or more vesicles in the population of vesicles between the lipid raft phase domain and the non-raft phase domain, optionally wherein the change is detected by comparing the signal with a signal from the population of vesicles detected before the contacting with the candidate compound; and/or detecting a change in at least one of the one or more membrane proteins from the lipid raft phase domain or the non-raft phase domain to the other phase domain, optionally wherein the change is detected by comparing the signal with a signal from the population of vesicles detected before the contacting with the candidate compound; and/or calculating Z-scores and ranking hits.

14. The method of claim 13, wherein detecting a change in phase separation comprising detecting a change in partition coefficients and/or a change in percent of phase separated vesicles in the population of vesicles.

15. The method of claim 14, wherein the measured partition coefficient is for the distribution of a fluorescently-labeled protein between lipid raft phase domain and non-raft phase domain.

16. The method of claim 1, comprising analyzing multiple vesicles in the population of vesicles across multiple reaction wells in an automated fashion.

17. A system for automatedly identifying the candidate compound as having an impact on a characteristic of a lipid raft phase domain, a characteristic of a non-raft phase domain, and/or a characteristic of one or more membrane proteins, the system comprising:
a plate comprising a plurality of reaction wells for contacting a population of vesicles with a candidate compound, wherein one or more vesicles in the population of vesicles optionally comprises one or more membrane proteins, wherein in a portion of the population of vesicles there is only a single detectable membrane phase and in a portion of the population of vesicles a membrane lipid raft phase domain and a membrane non-raft phase domain are phase separated; and
a computing platform including a processor and memory, the computing platform including a module configured to detect a signal from the population of vesicles using a microscope, camera or a sensor communicatively coupled to the computing platform; and to identify the candidate compound as having an impact on a characteristic of a lipid raft phase domain, a characteristic of a non-raft phase domain, and/or a characteristic of one or more membrane proteins based on the signal.

18. The system of claim 17, wherein the signal is used to assess whether the candidate compound induces changes in the percent of vesicles containing the single visible phase within the population of vesicles; changes in the relative sizes and/or characteristics of the lipid raft phase versus the non-raft phase domains; and/or changes in the distribution of one or more membrane proteins between the lipid raft phase and the non-raft phase domains.

19. The system of claim 17, wherein the characteristic of a lipid raft phase domain, a characteristic of a non-raft phase domain, and/or a characteristic of one or more membrane proteins comprises membrane phase behavior, partitioning of proteins between the lipid raft phase domain and the non-raft phase domain, or both.

20. The system of claim 17, wherein the vesicle comprises a Giant Plasma-Membrane Derived Vesicle (GPMV).

21. The system of claim 17, wherein the lipid raft phase domain, the non-raft phase domain, and/or the one or more membrane proteins are distinguishably labeled.

22. The system of claim 21, wherein (a) the lipid raft phase domain is labeled with a first fluorescent label and the non-raft phase domain is labeled with a second fluorescent label, wherein the first fluorescent label and the second fluorescent label are distinguishable from each other; (b) the lipid raft phase domain is labeled with a first fluorescent label and the one or more membrane proteins is labeled with a second fluorescent label, wherein the first fluorescent label and the second fluorescent label are distinguishable from each other; (c) the non-raft phase domain is labeled with a first fluorescent label and the one or more membrane proteins is labeled with one or more additional fluorescent labels, wherein the first fluorescent label and the additional fluorescent labels are all distinguishable from each other; and (d) the lipid raft phase domain is labeled with a first fluorescent label, the non-raft phase domain is labeled with a second fluorescent label, and the one or more membrane proteins is labeled with a third or more fluorescent label, wherein the first fluorescent label, the second fluorescent label, and the third or more fluorescent labels are all distinguishable from each other,

23. The system of claim 17, wherein detecting a signal in the population of vesicles further comprises detecting a detectable label in the population of vesicles, taking one or more images of one or more vesicles in the population of vesicles, or a combination thereof.

24. The system of claim 17, wherein detecting a signal from the population of vesicles comprises identifying one or more vesicles, excluding one or more vesicles that do not meet one or more selection criteria, mapping a position of membrane contour for each of the one or more vesicles, generating a plot of fluorescence intensity along the membrane of the vesicle, calculating a partition coefficient of a vesicle, scoring a vesicle as phase separated or not, or any combination of any of the foregoing.

25. The system of claim 24, wherein calculating a partition coefficient of a vesicle comprises calculating the partition coefficients of the fluorescent labels and/or fluorescently labeled membrane proteins as a function of angle around the entire vesicle.

26. The system of claim 24, wherein scoring a vesicle as phase separated or not comprises determining whether a histogram of the angular partition coefficient of a vesicle comprises a single peak centered around 0.5 or instead contains multiple peaks reflecting a presence of fluorophore-rich and fluorophore-poor domains.

27. The system of claim 24, wherein scoring a vesicle as phase separated or not comprises separating data points into two separate quadrants for vesicles having phase separated membranes and colocalizing datapoints on a diagonal for membranes containing a single uniform phase.

28. The system of claim 17, wherein the computing platform is configured for correcting the imaging for a position effect.

29. The system of claim 17, wherein identifying the candidate compound as having an impact on a characteristic of a lipid raft based on the signal comprises detecting a change in phase separation in one or more vesicles in the population of vesicles between the lipid raft phase domain and the non-raft phase domain, optionally wherein the change is detected by comparing the signal with a signal from the population of vesicles detected before the contacting with the candidate compound; and/or detecting a change in at least one of the one or more membrane proteins from the lipid raft phase domain or the non-raft phase domain to the other phase, optionally wherein the change is detected by comparing the signal with a signal from the population of vesicles detected before the contacting with the candidate compound; and/or calculating Z-scores and ranking hits.

30. The system of claim 29, wherein detecting a change in phase separation comprising detecting a change in partition coefficients and/or a change in percent of phase separated vesicles in the population of vesicles.

31. The system of claim 30, wherein the measured partition coefficient is for the distribution of a fluorescently-labeled protein between lipid raft and non-raft phase domains.

32. The system of claim 17, comprising analyzing multiple vesicles in the population of vesicles across multiple reaction wells in an automated fashion.

33. One or more non-transitory computer readable media having stored thereon executable instructions that when executed by one or more processors of one or more computers control the one or more computers to perform steps for automatedly identifying the candidate compound as having an impact on a characteristic of a lipid raft phase domain, a characteristic of a non-raft phase domain, and/or a characteristic of one or more membrane proteins based on the signal, the steps comprising:
contacting a population of vesicles with a candidate compound, wherein one or more vesicles in the population of optionally comprises one or more membrane proteins, with a candidate compound, wherein in a portion of the population of vesicles there is only a single detectable membrane phase and in a portion of the population of vesicles a membrane lipid raft phase domain and a membrane non-raft phase domain are phase separated;
detecting a signal from the population of vesicles; and
identifying the candidate compound as having an impact on a characteristic of a lipid raft phase domain, a characteristic of a non-raft phase domain, and/or a characteristic of one or more membrane proteins based on the signal.
